SEQUENCE LISTING (58 sequences)

SEQ ID NO: 1 - protein, 80 aa, Artificial Sequence
  CD24 full
MGRAMVARLGLGLLLLALLLPTQIYSSETTTGTSSNSSQSTSNSGLAPNPTNATTKAAGGALQSTASLFVVSLSLLHLYS

SEQ ID NO: 2 - protein, 32 aa, Artificial Sequence
  CD 24 extracellular domain
SETTTGTSSNSSQSTSNSGLAPNPTNATTKAA

SEQ ID NO: 3 - protein, 54 aa, Artificial Sequence
  CD24 Mature  human aa
SETTTGTSSNSSQSTSNSGLAPNPTNATTKAAGGALQSTASLFVVSLSLLHLYS

SEQ ID NO: 4 - DNA, 243 bp, Artificial Sequence
  CD24 human full nt
atgggcagagcaatggtggccaggctcgggctggggctgctgctgctggcactgctcctacccacgcagatttattccagtgaaacaacaactggaacttcaagtaactcctcccagagtacttccaactctgggttggccccaaatccaactaatgccaccaccaaggcggctggtggtgccctgcagtcaacagccagtctcttcgtggtctcactctctcttctgcatctctactcttaa

SEQ ID NO: 5 - protein, 292 aa, Artificial Sequence
  CD47 human full aa
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 6 - protein, 140 aa, Artificial Sequence
  CD47 extracellular domain human aa
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPN

SEQ ID NO: 7 - protein, 122 aa, Artificial Sequence
  CD47 mature extracellular domain human aa
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPN

SEQ ID NO: 8 - protein, 274 aa, Artificial Sequence
  CD47  mature human aa
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 9 - protein, 21 aa, Artificial Sequence
  CD47 peptide
GNYTCEVTELTREGETIIELK

SEQ ID NO: 10 - protein, 16 aa, Artificial Sequence
  VARIANT (1)...(1): Xaa is cysteine, threonine, or null
  VARIANT (14)...(14): Xaa is cysteine, threonine, or null
  CD47 peptide
XAAEVTELTREGEXAA

SEQ ID NO: 11 - protein, 10 aa, Artificial Sequence
  CD47 peptide
EVTELTREGE

SEQ ID NO: 12 - protein, 12 aa, Artificial Sequence
  CD47 peptide
CEVTELTREGEC

SEQ ID NO: 13 - DNA, 882 bp, Artificial Sequence
  CD47 full human nt
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggaataa

SEQ ID NO: 14 - protein, 148 aa, Artificial Sequence
  Human CD47 transmembrane domain
NILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYM

SEQ ID NO: 15 - protein, 129 aa, Artificial Sequence
  Human CD24
MVGRFCPESPPGFVRVAATSAVSLDPPSGEPRPGCGYPGPRSAASRVYGCTAPARETGGWAWETLAGAGAKKIYSSETTTGTSSNSSQSTSNSGLAPNPTNATTKAAGGALQSTASLFVVSLSLLHLYS

SEQ ID NO: 16 - protein, 129 aa, Artificial Sequence
  Bonobo CD24
MVGRFCPESPPGFVQVAATSAVSLDPPSGEPRPGCGYLGPRSAASRVYGCTAPARETGGWAWETLAGVGAKKIYSSETTTGTSSNSSQSTSNSGFAPNPTNATTKAAGGALQSTASLFVVSLSLLHLYS

SEQ ID NO: 17 - protein, 129 aa, Artificial Sequence; identical to SEQ ID NO: 16
  Chimp CD24

SEQ ID NO: 18 - protein, 32 aa, Artificial Sequence
  CD 24 extracellular domain aa
SETTTGTSSNSSQSTSNSGFAPNPTNATTKAA

SEQ ID NO: 19 - protein, 602 aa, Artificial Sequence
  NiVG protein attachment glycoprotein (602 aa)
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 20 - protein, 604 aa, Artificial Sequence
  Hendra Virus G Protein
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 21 - protein, 603 aa, Artificial Sequence
  Hendra Virus G Protein without Met
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 22 - protein, 602 aa, Artificial Sequence
  Nipah Virus G Protein
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 23 - protein, 601 aa, Artificial Sequence
  Nipah Virus G Protein (No Met)
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 24 - protein, 622 aa, Artificial Sequence
  Cedar Virus G Protein
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 25 - protein, 621 aa, Artificial Sequence
  Cedar Virus G Protein (No Met)
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 26 - protein, 632 aa, Artificial Sequence
  Bat Paramyxovirus G Protein
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 27 - protein, 631 aa, Artificial Sequence
  Bat Paramyxovirus G Protein (No Met)
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 28 - protein, 625 aa, Artificial Sequence
  Mojiang virus, Tongguan 1 G Protein
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 29 - protein, 624 aa, Artificial Sequence
  Mojiang virus, Tongguan 1 G (No Met)
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 30 - protein, 597 aa, `Artificial Sequence
  NiVG protein attachment glycoprotein, Truncated 5
MKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 31 - protein, 582 aa, Artificial Sequence
  NiVG protein attachment glycoprotein, Truncated 20
MSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 32 - protein, 577 aa, Artificial Sequence
  NiVG protein attachment glycoprotein, Truncated 25
MSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 33 - protein, 500 aa, Artificial Sequence
  Nipah virus NiV-F F0 T234 truncation (aa 525- 544) AND mutation on N-linked glycosylation site
ILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 34 - protein, 524 aa, Artificial Sequence
  Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 35 - protein, 569 aa, Artificial Sequence
  NiVG protein attachment glycoprotein Truncated and mutated (E501 A, W504A, Q530A, E533A) NiV G protein (Gc 34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 36 - protein, 568 aa, Artificial Sequence
  NiVG protein attachment glycoprotein Truncated and mutated (E501 A, W504A, Q530A, E533A) NiV G protein (Gc 34) Without N-terminal methionine
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 37 - protein, 524 aa, Artificial Sequence; identical to SEQ ID NO: 34
  Truncated NiV fusion glycoprotein (FcDelta22) at cytoplasmic tail (with signal sequence)

SEQ ID NO: 38 - protein, 500 aa, Artificial Sequence
  Nipah virus NiV-F F0 T234 truncation (aa 525- 544)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 39 - protein, 498 aa, Artificial Sequence
  Truncated  mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 40 - protein, 552 aa, Artificial Sequence
  NivG protein attachment glycoprotein Without cytoplasmic tail Uniprot Q9IH62
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 41 - protein, 604 aa, Artificial Sequence; identical to SEQ ID NO: 20
  Hendra virus G protein Uniprot O89343

SEQ ID NO: 42 - protein, 603 aa, Artificial Sequence; identical to SEQ ID NO: 21
  Hendra virus G protein Uniprot O89343 Without N-terminal methionine

SEQ ID NO: 43 - protein, 555 aa, Artificial Sequence
  Hendra virus G protein Uniprot O89343, Without cytoplasmic tail
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 44 - protein, 555 aa, Artificial Sequence; identical to SEQ ID NO: 43
  Hendra virus G protein Uniprot O89343

SEQ ID NO: 45 - protein, 602 aa, Artificial Sequence; identical to SEQ ID NO: 19
  NiVG protein attachment glycoprotein (602 aa)

SEQ ID NO: 46 - protein, 546 aa, Artificial Sequence
  Hendra virus F Protein
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 47 - protein, 520 aa, Artificial Sequence
  Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 48 - protein, 546 aa, Artificial Sequence
  Nipah virus F Protein
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 49 - protein, 520 aa, Artificial Sequence
  Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 50 - protein, 557 aa, Artificial Sequence
  Cedar Virus F Protein
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 51 - protein, 551 aa, Artificial Sequence
  Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 52 - protein, 545 aa, Artificial Sequence
  Mojiang virus, Tongguan 1 F Protein
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 53 - protein, 519 aa, Artificial Sequence
  Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 54 - protein, 662 aa, Artificial Sequence
  Bat Paramyxovirus F Protein
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 55 - protein, 640 aa, Artificial Sequence
  Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 56 - protein, 26 aa, Artificial Sequence
  signal sequence
MVVILDKRCYCNLLILILMISECSVG

SEQ ID NO: 57 - protein, 83 aa, Artificial Sequence
  Nipah virus NiV-F F2 (aa 27-109)
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 58 - protein, 546 aa, Artificial Sequence; identical to SEQ ID NO: 48
  Nipah virus F Protein

(19) United States
(12) Patent Application Publication
(10) Pub. No.: US 2022/0333132 A1
(43) Pub. Date: Oct. 20, 2022

(54) CD24-ASSOCIATED PARTICLES AND RELATED METHODS AND USES THEREOF

(71) Applicant: Sana Blotechnology, Inc., Seattle, WA (US)

(72) Inventors: Akinola Olumide EMMANUEL, Cambridge, WA (US); Hanane ENNAJDAOUI, Cambridge, MA (US); Suvi JAIN, Waltham, MA (US); Alberto RUZO MATIAS, Brookline, MA (US); Jagesh V. SHAHA, Lexington, MA (US)

(73) Assignee: Sana Blotechnology, Inc., Seattle, WA (US)

(21) Appl. No.: 17/639,843

(22) PCT Filed: Sep. 2, 2020
(86) PCT No.: PCT/US2020/049087

§ 371 Date: Mar. 2, 2022

Related U.S. Application Data
(60) Provisional application No. 63/056,514, filed on Jul. 24, 2020.
(60) Provisional application No. 62/895,454, filed on Sep. 3, 2019.

(51) Int. Cl. C12N 15/86; C07K 14/705

(52) U.S. Cl. CPC C12N 15/86; C07K 14/70596; C12N 2740/15043; C12N 2740/15023; C12N 2740/15071; C12N 2740/15052

(57) ABSTRACT

Provided herein are non-cell particles, e.g. virus particles or virus-like particles, such as pseudotyped lentiviral-like particles, containing an exogenous CD24 or a biologically active portion of CD24. In some embodiments, the non-cell particles, e.g. virus particles or virus-like particles, such as pseudotyped lentiviral-like particles, can further contain an exogenous CD47 or a biologically active portion of CD47. Also provided herein are compositions containing such non-cell particles and methods of making and using the non-cell particles.

### CROSS-REFERENCE TO RELATED APPLICATIONS

This application claims priority to U.S. provisional application 62/895,454, filed Sep. 3, 2020, and to U.S. provisional application 63/056,514, filed Jul. 24, 2020, the contents of each of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 186152003740SeqList.TXT, created Sep. 2, 2020, which is 202,892 bytes in size. The information in the electronic format of the Sequence Listing is incorporated by reference in its entirety.

### FIELD

The present disclosure relates to non-cell particles, e.g. virus particles or virus-like particles, such as pseudotyped lentiviral-like particles, containing an exogenous CD24 or a biologically active portion of CD24. In some embodiments, the non-cell particles, e.g. virus particles or virus-like particles, such as pseudotyped lentiviral-like particles, can further contain an exogenous CD47 or a biologically active portion of CD47. The present disclosure also provides compositions containing such non-cell particles and methods of making and using the non-cell particles.

### BACKGROUND

Non-cell particles, such as lentiviral-like vector particles, are commonly used in gene therapy for delivery of exogenous agents to cells. When administered to subjects, however, such particles can be recognized as foreign and can be engulfed or scavenged by phagocytic cells.

Phagocytosis is conducted primarily by highly specialized cells, such as macrophages, monocytes and neutrophils, with the goal of clearing pathogens and/or other foreign invaders that the phagocytic cell recognizes through an array of specialized pattern recognition receptors. Phagocytosis of particles, such as lentiviral vector particles, limits the in vivo half-life and their effectiveness as gene therapy vectors. Thus, there is a long felt need for effective particle delivery vehicles, including lentiviral-like particles along with other types of non-cell particles, for effectively evading the phagocytic cell uptake of the immune system, thereby resulting in a reduction of an immune response. The provided disclosure addresses this need.

### SUMMARY

Provided herein is a non-cell particle comprising CD24 or a biologically active portion thereof on an exposed surface of the particle, wherein the non-cell particle is 1 μm or smaller. In some embodiments, the CD24 or the biologically active portion thereof binds Siglec-10. In some embodiments, the CD24 or biologically active portion thereof is human.

In some embodiments, the CD24 or biologically active portion thereof (i) comprises the sequence set forth in SEQ ID NO: 2; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds Siglec-10; or (iii) a binding portion of (i) or (ii) that binds to Siglec-10. In some embodiments, the CD24 or biologically active portion thereof comprises the sequence set forth in SEQ ID NO:2.

In some embodiments, the CD24 or biologically active portion is displayed on an exposed surface of the particle via a Glycosylphosphatidylinositol (GPI) membrane anchor.

In some embodiments, the CD24 or the biologically active portion thereof is encoded by a nucleic molecule encoding the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds Siglec-10. In some embodiments, the CD24 or the biologically active portion thereof is encoded by a nucleic molecule encoding the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds Siglec-10.

In some embodiments, the CD24 or biologically active portion is displayed on an exposed surface of the particle via a transmembrane domain.

In some embodiments, the CD24 or biologically active portion is a glycoprotein. In some embodiments, the CD24 or biologically active portion is sialylated. In some embodiments, the CD24 or biologically active portion comprises an α2-3-linked sialoside and/or an α2-6-linked sialoside. In some embodiments, the CD24 or biologically active portion has a molecular weight of between at or about 35 kDa and at or about 45 kDa.

In some embodiments, the non-cell particle further comprises a CD47 or a biologically active portion thereof on an exposed surface of the non-cell particle. In some embodiments, the CD47 or biologically active portion binds to SIRPα. In some embodiments, the CD47 or biologically active portion is human. In some embodiments, the CD47 or biologically active portion comprises the extracellular domain or CD47 or a binding portion thereof that binds to SIRPα.

In some embodiments, the CD47 or biologically active portion thereof (i) comprises the sequence set forth in SEQ ID NO: 7; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or (iii) a binding portion of (i) or (ii) that binds to SIRPα. In some embodiments, the CD47 or biologically active portion thereof comprises the sequence set forth in SEQ ID NO:7. In some embodiments, the CD47 or biologically active portion thereof comprises the sequence set forth in SEQ ID NO:11. In some embodiments, the CD47 or biologically active portion thereof comprises the sequence set forth in SEQ ID NO:9. In some embodiments, the CD47 or biologically active portion thereof comprises the sequence set forth in SEQ ID NO:10. In some embodiments, the CD47 or biologically active portion thereof comprises the sequence set forth in SEQ ID NO:12.

In some embodiments, the CD47 or biologically active portion is displayed on an exposed surface of the non-cell particle via a transmembrane domain.

In some embodiments, the CD47 or biologically active portion comprises SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα. In some embodiments, the CD47 or the biologically active portion thereof is encoded by a nucleic molecule encoding the sequence set forth in SEQ ID NO:5 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5 that binds to SIRPα.

In some embodiments, the non-cell particle further comprises an exogenous agent.

In some embodiments, the non-cell particle further comprises a nucleic acid encoding an exogenous agent. In some embodiments, the exogenous agent is a protein.

In some embodiments, the non-cell particle is a synthetic particle, a viral particle or a cell-derived particle. In some embodiments, the non-cell particle is a viral-based particle or a non-viral particle. In some embodiments, the non-cell particle further comprises an exogenous agent. The exogenous agent can be present in the lumen of the non-cell particle. In some embodiments, the exogenous agent is a protein and is present in the lumen as a protein. In some embodiments, the exogenous agent is encoded by a nucleic acid, and a nucleic acid is a payload gene encoding the exogenous agent. In some embodiments, the non-cell particle is a synthetic particle selected from the group consisting of a liposome, a microparticle, a nanoparticle, a nanogel, a dendrimer and a dendrisome. In some embodiments, the non-cell particle is a non-viral particle selected from the group consisting of a liposome, a microparticle, a nanoparticle, a nanogel, a dendrimer and a dendrisome

In some embodiments, the non-cell particle is a synthetic particle, a viral particle or a cell-derived particle. In some embodiments, the non-cell particle is a viral-based particle or a non-viral particle. In some embodiments, the non-cell particle further comprises a nucleic acid comprising a payload gene encoding an exogenous agent. In some embodiments, the non-cell particle is a synthetic particle selected from the group consisting of a liposome, a microparticle, a nanoparticle, a nanogel, a dendrimer and a dendrisome. In some embodiments, the non-cell particle is a non-viral particle selected from the group consisting of a liposome, a microparticle, a nanoparticle, a nanogel, a dendrimer and a dendrisome

In some embodiments, the exposed surface is a lipid bilayer and the non-cell particle further comprises a lumen comprising a cytosol, wherein the lumen is surrounded by the lipid bilayer. In some embodiments, the lumen further comprises a nucleic acid comprising a payload gene encoding an exogenous agent. In some embodiments, the non-cell particle is a fusosome and the lipid bilayer further comprises a fusogen.

In some embodiments, the non-cell particle is derived from a source cell. In some embodiments, the non-cell particle does not comprise a nucleus.

In some embodiments, the non-cell particle is a viral-based particle In some embodiments, the non-cell particle is a virus particle or a virus-like particle (VLP). In some embodiments, the virus particle or virus-like particle is a retroviral particle or retrovirus-like particle. In some embodiments, the retroviral particle or retrovirus-like particle is a lentiviral particle or a lentiviral-like particle.

In some embodiments, the non-cell particle comprises a fusogen that is a viral fusogen selected from a Class I viral membrane fusion protein, a Class II viral membrane protein, a Class II viral membrane fusion protein, a viral membrane glycoprotein, or a viral envelope protein. In some embodiments, the fusogen is endogenous to the virus. In some embodiments, the fusogen is a pseudotyped fusogen. In some embodiments, the fusogen is a re-targeted fusogen that binds to a target cell. In some embodiments, the fusogen comprises a targeting moiety that binds to the target cell.

In some embodiments, the virus or virus-like particle further comprises a lumen comprising a nucleic acid. In some embodiments, the nucleic acid comprises a viral nucleic acid comprising one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3). In some embodiments, the non-cell particle is a virus-like particle (e.g. retrovirus-like particle) that is a replication defective. In some embodiments, the non-cell particle is a viral particle (e.g. retroviral particle) that is a replication defective.

Provided herein is a pseudotyped viral-based particle, e.g., lentivirus or lentiviral-like particle, comprising CD24 or a biologically active portion thereof on an exposed surface of the lentiviral particle. In some embodiments, the particle is pseudotyped with a vesicular stomatitis virus envelope glycoprotein (VSV-G). In some embodiments, the viral-based particle, e.g., lentiviral particle, is pseudotyped with a protein from an envelope glycoprotein of a virus of the Paramyxovirus family or is a biologically active portion thereof. In some embodiments, the virus of the Paramyxovirus family is a Henipavirus or is a Morbillivirus. In some embodiments, the envelope glycoprotein is an envelope glycoprotein G or H or is a biologically active portion thereof. In some embodiments, the non-cell particle is pseudotyped with a Nipah virus G (Niv-G) protein. or is a biologically active portion thereof. In some embodiments, the non-cell particle is pseudotyped with a Hendra virus G protein. In some embodiments, the non-cell particle is pseudotyped with a measles virus glycoprotein.

In some embodiments, the non-cell particle is pseudotyped with a cell targeting fusion protein comprising a Paramyxoviridae envelope protein derived from an envelope glycoprotein G or H and at least one cell targeting domain. In some embodiments, said protein derived from an envelope glycoprotein G or H or a biologically active portion thereof of a virus of the Paramyxoviridae family is at least partially unable to bind at least one natural receptor of said envelope glycoprotein G or H. For instance, in some cases the envelope glycoprotein G or H or a biologically active portion thereof may contain one or more mutations that interfere with natural receptor recognition. In some embodiments, the virus of the Paramyxovirus family is a Henipavirus or is a Morbillivirus. In some embodiments, the envelope glycoprotein is a Nipah virus G (Niv-G) protein. In some embodiments, the envelope glycoprotein is a Hendra virus G protein.

In some of any of the provided embodiments, the non-cell particle further contains an F protein molecule or a biologically active portion thereof from a Paramyxovirus. In some embodiments, the non-cell particle contains (1) an F protein molecule or a biologically active portion thereof from a Paramyxovirus and (2) a glycoprotein G or H protein or a biologically active portion thereof from a Paramyxovirus, or a cell targeting fusion protein thereof containing the glycoprotein G or H protein or biologically active portion thereof and a cell targeting moiety. In some of any of the provided embodiments, the Paramyxovirus is a henipavirus. In some of any of the provided embodiments, the Paramyxovirus is Nipah virus. In some of any of the provided embodiments, the Paramyxovirus is Hendra virus. In some embodiments, the F protein or a biologically active portion thereof is a Niv-F or a biologically active portion thereof.

In some embodiments, the CD24 or biologically active portion thereof (i) comprises the sequence set forth in SEQ ID NO: 2; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds to Siglec-10; or (iii) a binding portion of (i) or (ii) that binds to Siglec-10. In some embodiments, the CD24 or biologically active portion thereof comprises the sequence set forth in SEQ ID NO:2.

In some embodiments, the CD24 or biologically active portion is displayed on an exposed surface of the non-cell particle via a Glycosylphosphatidylinositol (GPI) membrane anchor.

In some embodiments, the CD24 or the biologically active portion thereof is encoded by a nucleic molecule encoding the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds to Siglec-10. In some embodiments, the CD24 or the biologically active portion thereof is encoded by a nucleic molecule encoding the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds to Siglec-10.

In some embodiments, the CD24 or biologically active portion is displayed on an exposed surface of the non-cell particle via a transmembrane domain. In some embodiments, the CD24 or biologically active portion is a glycoprotein. In some embodiments, the CD24 or biologically active portion is sialylated. In some embodiments, the CD24 or biologically active portion comprises an α2-3-linked sialoside and/or an α2-6-linked sialoside. In some embodiments, the CD24 or biologically active portion has a molecular weight of between at or about 35 kDa and at or about 45 kDa.

In some embodiments, the non-cell particle further comprises a CD47 or a biologically active portion thereof on an exposed surface of the non-cell particle. In some embodiments, the CD47 or biologically active portion binds to SIRPα. In some embodiments, the CD47 or biologically active portion is human. In some embodiments, the CD47 or biologically active portion comprises the extracellular domain or CD47 or a binding portion thereof that binds to SIRPα.

In some embodiments, the CD47 or biologically active portion thereof (i) comprises the sequence set forth in SEQ ID NO: 7; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or (iii) a binding portion of (i) or (ii) that binds to SIRPα. In some embodiments, the CD47 or biologically active portion thereof comprises the sequence set forth in SEQ ID NO:7. In some embodiments, the CD47 or biologically active portion thereof comprises the sequence set forth in SEQ ID NO:11. In some embodiments, the CD47 or biologically active portion thereof comprises the sequence set forth in SEQ ID NO:9. In some embodiments, the CD47 or biologically active portion thereof comprises the sequence set forth in SEQ ID NO:10. In some embodiments, the CD47 or biologically active portion thereof comprises the sequence set forth in SEQ ID NO:12.

In some embodiments, the CD47 or biologically active portion is displayed on an exposed surface of the particle via a transmembrane domain.

In some embodiments, the CD47 or biologically active portion comprises SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα. In some embodiments, the CD47 or the biologically active portion thereof is encoded by a nucleic molecule encoding the sequence set forth in SEQ ID NO:5 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5 that binds to SIRPα.

In some embodiments, the non-cell particle or the pseudotyped non-cell particle, e.g., lentivirus or lentiviral-like particle, further comprises a nucleic acid comprising a payload gene encoding an exogenous agent. In some embodiments, the exogenous agent encodes a therapeutic agent or a diagnostic agent.

In some embodiments, phagocytosis of the non-cell particle by a phagocytic cells, optionally a macrophage, is reduced compared to a reference particle that is otherwise similar but does not comprise the CD24 or biologically active portion, optionally wherein phagocytosis is reduced by at least 10%, 20%, 30%, 40%, 50%, 60%, 70%, 80%, or 90%. In some embodiments, the half-life of the non-cell particle in vivo is increased compared to a reference particle that is otherwise similar but does not comprise the CD24 or biologically active portion, optionally wherein the half-life is increased by at least 10%, 20%, 30%, 40%, 50%, 60%, 70%, 80%, or 90%.

Provided herein is a polynucleotide comprising a first nucleic acid sequence encoding CD24 or a biologically active portion and a second nucleic acid encoding CD47 or a biologically active portion. In some embodiments, the encoded CD24 or the biologically active portion thereof binds Siglec-10. In some embodiments, the encoded CD24 or biologically active portion thereof is human.

In some embodiments, the encoded CD24 or biologically active portion thereof (i) comprises the sequence set forth in SEQ ID NO: 2; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds to Siglec-10; or (iii) a binding portion of (i) or (ii) that binds to Siglec-10. In some embodiments, the encoded CD24 or biologically active portion thereof comprises the sequence set forth in SEQ ID NO:2.

In some embodiments, the encoded CD24 or biologically active portion is displayed on an exposed surface of the particle via a Glycosylphosphatidylinositol (GPI) membrane anchor.

In some embodiments, the first nucleic acid encoding CD24 or a biologically active portion encodes the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds to Siglec-10; or the first nucleic acid encoding CD24 or a biologically active portion encodes the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds to Siglec-10. In some embodiments, the first nucleic acid encoding CD24 comprises the sequence set forth in SEQ ID NO:4 or a sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:4.

In some embodiments, the encoded CD47 or biologically active portion binds to SIRPα. In some embodiments, the encoded CD47 or biologically active portion is human. In some embodiments, the encoded CD47 or biologically active portion comprises the extracellular domain or CD47 or a binding portion thereof that binds to SIRPα.

In some embodiments, the encoded CD47 or biologically active portion thereof (i) comprises the sequence set forth in SEQ ID NO: 7; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or (iii) a binding portion of (i) or (ii) that binds to SIRPα. In some embodiments, the encoded CD47 or biologically active portion thereof comprises the sequence set forth in SEQ ID NO:7. In some embodiments, the encoded CD47 or biologically active portion thereof comprises the sequence set forth in SEQ ID NO:11. In some embodiments, the encoded CD47 or biologically active portion thereof comprises the sequence set forth in SEQ ID NO:9. In some embodiments, the encoded CD47 or biologically active portion thereof comprises the sequence set forth in SEQ ID NO:10. In some embodiments, the encoded CD47 or biologically active portion thereof comprises the sequence set forth in SEQ ID NO:12.

In some embodiments, the encoded CD47 or biologically active portion comprises a transmembrane domain.

In some embodiments, the second nucleic acid encodes the sequence set forth in SEQ ID NO: 5 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:5 that binds to SIRPα; or the second nucleic acid encodes the sequence set forth in SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα. In some embodiments, the second nucleic acid encoding CD47 comprises the sequence set forth in SEQ ID NO:13 or a sequence having at least at or about 90%, at least at or about 91%, at or about 92%, at least at or about 93%, at least at or about 94%, at or about 95%, at least at or about 96%, at least at or about 97%, at least at or about 98%, or at least at or about 99% identity to SEQ ID NO:13.

In some embodiments, the polynucleotide further comprises at least one promoter that is operatively linked to control expression of the CD24 or biologically active portion and/or the CD47 or biologically active portion. In some embodiments, the first and second nucleic acid are operatively linked to the same promoter. In some embodiments, the first nucleic acid is operatively linked to a first promoter and the second nucleic acid is operatively linked to a second promoter. In some embodiments, the first and second promoter are different. In some embodiments, the promoter, or each promoter individually, is a heterologous promoter. In some embodiments, the promoter, or each promoter individually, is an inducible promoter.

In some embodiments, the polynucleotide further comprises a nucleic acid sequence encoding a linking peptide between the first and second nucleic acid sequences, wherein the linking peptide separates the translation products of the first and second nucleic acid sequences during or after translation. In some embodiments, the linking peptide comprises an internal ribosome entry site (IRES), a self-cleaving peptide, or a peptide that causes ribosome skipping, optionally a T2A peptide.

Provided herein is a vector comprising the polynucleotide of any of the preceding embodiments. In some embodiments, the vector is a mammalian vector, viral vector or artificial chromosome, optionally wherein the artificial chromosome is a bacterial artificial chromosome (BAC).

Provided herein is a method of making a non-cell particle comprising CD24 or a biologically active portion, comprising a) providing a cell that comprises a nucleic acid encoding CD24 or a biologically active portion thereof; b) culturing the cell under conditions that allow for production of a non-cell particle, and c) separating, enriching, or purifying the non-cell particle from the cell, thereby making a non-cell particle comprising CD24 or a biologically active portion. In some embodiments, the non-cell particle further comprises a fusogen. In some embodiments, the non-cell particle is a fusosome. In some embodiments, the cell further comprises a nucleic acid encoding CD47 or a biologically active portion thereof or the nucleic acid further encodes CD47 or a biologically active portion thereof.

Provided herein is a method of making a non-cell particle comprising CD24 or a biologically active portion, comprising a) providing a cell that comprises the polynucleotide or the vector of any of the preceding embodiments; b) culturing the cell under conditions that allow for production of a non-cell particle, and c) separating, enriching, or purifying the non-cell particle from the cell, thereby making a non-cell particle comprising CD24 or a biologically active portion. In some embodiments, the non-cell particle further comprises a fusogen. In some embodiments, the non-cell particle is a fusosome.

In some embodiments, the cell is a mammalian cell and the non-cell particle is a vesicle or an exosome. In some embodiments, the vesicle is a microvesicle or a nanovesicle. In some embodiments, the cell is a producer cell and the non-cell particle is a viral particle or a viral-like particle. In some embodiments, the viral particle or viral-like particle is a retroviral particle or a retroviral-like particle. In some embodiments, the viral particle or viral-like particle is a lentiviral particle or lentiviral-like particle. In some embodiments, the cell further comprises an exogenous nucleic acid sequence encoding an exogenous agent. In some embodiments, the cell further comprises a fusogen.

Provided herein is a non-cell particle made by the method of any of the preceding embodiments.

Provided herein is a mammalian cell comprising (i) a viral nucleic acid(s) and (ii) nucleic acid encoding an exogenous CD24 or a biologically active portion thereof, optionally wherein the viral nucleic acid(s) are lentiviral nucleic acids. In some embodiments, the viral nucleic acid(s) lacks one or more genes involved in viral replication. In some embodiments, the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3); a nucleic acid encoding a viral envelope protein; and/or a nucleic acid encoding a viral packaging protein selected from one or more of Gag, Pol, Rev and Tat. In some embodiments, the exogenous CD24 or biologically active portion comprises (i) the sequence set forth in SEQ ID NO: 2; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds Siglec-10; or (iii) a binding portion of (i) or (ii) that binds to Siglec-10. In some embodiments, the nucleic acid encoding exogenous CD24 further encodes a Glycosylphosphatidylinositol (GPI) membrane anchor signal sequence or a transmembrane domain.

In some embodiments, the cell further comprises a nucleic acid encoding exogenous CD47 or a biologically active portion. In some embodiments, the exogenous CD47 or biologically active portion comprises (i) the sequence set forth in SEQ ID NO: 7; (ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or (iii) a binding portion of (i) or (ii) that binds to SIRPα. In some embodiments, the nucleic acid encoding exogenous CD47 further encodes a Glycosylphosphatidylinositol (GPI) membrane anchor signal sequence or a transmembrane domain.

In some embodiments, the nucleic acid encoding the exogenous CD24 or biologically active portion and the nucleic acid encoding the exogenous CD47 or biologically active portion are encoded by the polynucleotide of any of the preceding embodiments.

Provided herein is a non-cell particle that is viral-based, e.g. a viral vector particle or a viral-like particle, produced from the mammalian cell of any of the preceding embodiments.

Provided herein is a composition comprising a plurality of non-cell particles of any of the preceding embodiments.

Provided herein is a composition comprising a plurality of pseudotyped lentivirus or lentiviral-like particles of any of the preceding embodiments.

In some embodiments, the composition further comprises a pharmaceutically acceptable carrier. In some embodiments, the plurality of particles comprise an average diameter of less than 1 μm.

Provided herein is a method of delivering an exogenous agent to a subject (e.g., a human subject), the method comprising administering to the subject the non-cell particle, the pseudotyped lentivirus or lentiviral-like particles, or the composition of any of the preceding embodiments.

Provided herein is a method of treating a disease or disorder in a subject (e.g., a human subject), the method comprising administering to the subject the non-cell particle, the pseudotyped lentivirus or lentiviral-like particles, or the composition of any of the preceding embodiments.

Provided herein is a method of evading phagocytosis of a particle by a phagocytic cell, the method comprising contacting a phagocytic cell with a non-cell particle, the pseudotyped lentivirus or lentiviral-like particles, or the composition of any of the preceding embodiments, whereby said particles evades phacocytosis by said phagocytic cell.

Provided herein is a method of increasing the life of a non-cell particle in vivo in a mammal, method comprising administering the non-cell particle, the pseudotyped lentivirus or lentiviral-like particles, or the composition of any of the preceding embodiments to a mammalian subject wherein said administered non-cell particles have a longer half-life in said mammal than an otherwise similar particle that does not have CD24 expressed thereon.

In some of any embodiments, the exogenous agent is a protein. In some embodiments, the exogenous agent is a membrane protein. In some embodiments, the exogenous agent is a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the exogenous agent is a CAR comprising an antigen binding domain, a transmembrane domain, and one or more signaling domains. In some embodiments, the antigen binding domain binds to a surface antigen characteristic of a cell type or a disorder. In some embodiments, the antigen binding domain binds to a surface antigen characteristic of a neoplastic cell, a T cell, an autoimmune or inflammatory disorder, a senescent cell, or an infectious disease.

Provided herein is a method of delivering an exogenous agent to a subject (e.g., a human subject), the method comprising administering to a subject a non-cell particle of any of the preceding embodiments, in which the non-cell particle contains the exogenous agent or a nucleic acid payload gene encoding the exogenous agent, and wherein the payload gene encoding the exogenous agent or the exogenous agent is delivered to a target cell. In some embodiments, the exogenous agent is a CAR. In some embodiments, the target cell is a T cell. In some embodiments, the target cell is any of a CD4+T cell, a CD8+T cell, an alpha beta T cell, a gamma delta T cell, a naive T cell, an effector T cell, a cytotoxic T cell (e.g., a CD8+ cytotoxic T cell), a regulatory T cell (e.g., a thymus-derived regulatory T cell, a peripherally derived regulatory T cell, a CD4+Foxp3+ regulatory T cell, or a CD4+FoxP3− type 1 regulatory T (Tr1) cell), a helper T cell (e.g., a CD4+ helper T cell, a Th1 cell, a Th2 cell, a Th3 cell, a Th9 cell, a Th17 cell, a Th22 cell, or a T follicular helper (Tfh) cell), a memory T cell (e.g., a stem cell memory T cell, a central memory T cell, or an effector memory T cell), a NKT cell, and a Mucosal associated invariant T (MAIT) cell.

### DETAILED DESCRIPTION

Provided herein are non-cell particles, such as viral-based particles (e.g. lentiviral and lentiviral-like particles), and including non-viral based non-cell particles such as fusosomes, containing CD24 or a biologically active portion thereon on an exposed surface of the non-cell particle (hereinafter also called “CD24-associated non-cell particles”). Also provided herein are CD24-associated non-cell particles, viral-based particles (e.g. lentiviral and lentiviral-like particles), and including non-viral based non-cell particles such as including fusosomes, further containing CD47 or a biologically active portion thereon an exposed surface of the non-cell particle. In particular embodiments, the non-cell particles are particles that are 1 μm or less in size.

CD24 (Cluster Differentiation 24) molecule or heat stable antigen (HAS) has been described as a cell adhesion molecule at the surface of many cell types including B cells or breast cancer cells for example. CD24 also has been shown to be a molecule in the evasion of breast cancer cells from phagocytosis (Barkal et al. 2019 Nature, 572:392-396). The human cell surface antigen CD24 is a sialoglycoprotein that is anchored to the cell surface by a glycosyl phosphatidylinositol (GPI) linkage (Van der Schoot et al., 1989, “Identification of three novel Pi-linked proteins on granulocytes” In: Knapp, W, et al. “Leukocyte Typing IV: White Cell Differentiation Antigens” Oxford Univ. Press p887-891; Fischer et al., 1990 J. Immun. 144: 638-641). Human CD24 is expressed as a precursor protein (e.g. set forth in SEQ ID NO:1; see also Uniprot No. P25063) containing a signal peptide (e.g. corresponding to amino acids 1-26 of SEQ ID NO:1) and a glycosylphosphatidylinositol (GPI) anchor cleavage sequence that is cleaved to result in a mature CD24 protein (e.g. set forth in SEQ ID NO:2) anchored to the membrane via a GPI anchor. CD24 is a glycoprotein that contains 16 potential O- and N-glycyosylation sites. In particular, CD24 has been shown to be sialylated, and α2-3 and α2-6-linked sialosides may be involved in its binding to the sialic acid-based recognition receptor Siglec 10 (receptor sialic-binding Ig-like lectin 10) (Chen et al. 2011 Nat. Biotechnol., 29:428-435). CD24 interacts with Siglec 10 on macrophages to inhibit phagocytosis. In particular embodiments, the provided CD24-associated non-cell particles exhibit reduced phagocytosis by scavenger cells, such as macrophages and/or increased half-life in circulation following administration in vivo.

Phagocytosis is a form of endocytosis wherein solid particles such as bacteria are engulfed by the cell membrane to form an internal phagosome. Phagocytosis is distinct from other forms of endocytosis, such as the vesicular internalization of liquids. Phagocytosis is a key mechanism used by the immune system to remove pathogens, cell debris, dead tissue cells and small mineral particles from circulation in the body.

Phagocytes are white blood cells that protect the body by phagocytosing harmful foreign particles, bacteria, and dead or dying cells, and thus are essential for fighting infections and developing subsequent immunity. Phagocytes of humans and other animals are called “professional” or “non-professional” depending on how effective they are at phagocytosis (Ernst & Stendahl, 2006, “Phagocytosis of Bacteria and Bacterial Pathogenicity”, Cambridge University Press: NY, p. 186). The distinguishing factor between professional and non-professional phagocytes is that professional phagocytes (such as neutrophils, monocytes, macrophages, dendritic cells, and mast cells) have surface receptors that can detect harmful objects, such as bacteria, that are not normally found in the body. The immune system recognizes invading cells (such as microbes and viruses) as foreign because these invading cells either express determinants that are absent on host cells or do not express “markers of self” that are normally present on host cells. Human macrophages internalize particles through discrete signals initiated from extracellular communications with a target resulting in cytoskeletal remodeling (Kovacs, M., et al., (2003). J Biol Chem 278(40): 38132-40) and contractile forces (Allen, L. H. et al., (1995). J. Exp. Med. 182(3): 829-840; Swanson, J. A., et al., (1999). J Cell Sci 112(3): 307-316) leading to engulfment.

In some cases, phagocytes may also attack elements that have been intentionally introduced into the body, such as implants, artificial tissue, artificial organs and vesicles bearing therapeutic agents, and this may reduce their lifetime in the body. For example, injected or implanted materials are also often perceived as foreign as these invariably activate macrophages and other phagocytes. This foreign body response can occur in spite of synthetic coatings, such as those with polyethylene glycol (PEG) that are intended to maximize compatibility. Non-cell particles, such as nanoparticles and viral-based particles, are often utilized to for delivery of exogenous agent (e.g. proteins or nucleic acids) for use as therapeutic agents or for diagnostic or imaging purposes. Although such non-cell particles are sufficiently small to avoid passive entrapment by capillaries in vivo, scavenger phagocytic cells (e.g. macrophages) in the spleen, liver or other site can clear such particles quickly, often within hours or days of injection into the circulation. Thus, improved methods are needed to reduce phagocytosis and/or increase half-life of administered particles.

In some cases, efforts to reduce phagocytosis can be achieved by targeting the inhibitory phagocyte receptor SIRP-alpha with CD47 (see e.g. U.S. Pat. No. 9,050,269). CD47 is a ubiquitous member of the Ig superfamily that interacts with the immune inhibitory receptor SIRPα(signal regulatory protein) found on macrophages (Fujioka et al., 1996, Mol. Cell. Biol. 16(12):6887-99; Veillette et al., 1998, J. Biol. Chem. 273(35):22719-28; Jiang et al., 1999, J. Biol. Chem. 274(2):559-62). CD47 has been well-documented as a ‘Marker of Self’ that can inhibit phagocytosis by macrophages (Lindberg, F. P., et al., (1994). J Biol Chem 269(3): 1567-70). CD47 in mouse (Oldenborg, P. A., et al., (2000). Science. 288(5473): 2051-4; Gardai, S. J., et al. (2005). 123(2): 321) and humans (Tsai, R. K. et al., (2008). J Cell Biol 180(5): 989-1003).

In certain contexts, however, targeting SIRP-alpha with CD47 may not always be completely satisfactory or sufficient to reduce phagocytosis. It has been shown that blockade of CD24 can result in a greater ability to induce phagocytosis than blockade of CD47 in certain cell types, which indicates superior activity of CD24 in certain contexts (Barkal et al. 2019 Nature, 572:392-396). The provided embodiments leverage the role of CD24 as an inhibitory phagocytic target to shield delivered particles, such as viral vectors and fusosomes, from being captured and removed by scavenger cells such as macrophages. Thus, the provided CD24-associated non-cell particles provide an alternative approach for reducing phagocytosis and/or increasing half-life of non-cell particles, particularly those less than 1 μm in size. In aspects of the provided embodiments, CD24 or a biologically active portion thereof that binds Siglec-10 is exposed on the surface of a non-cell particle and can thereby interact with Siglec-10 on scavenger cells (e.g. macrophages) to reduce phagocytosis. In some embodiments, the provided non-cell particles are engineered or produced to use CD24 as a detargeting molecule. In other embodiments, a dual detargeting approach is used in which non-cell particles are engineered or produced to use both CD24 and additionally CD47 as a detargeting molecule. For example, also provided herein are non-cell particles in which both CD24 or a biologically active portion thereof and CD47 or a biologically active portion thereof is exposed on the surface of a non-cell particle, thereby providing for dual recognition of phagocytic inhibitory receptors Siglec-10 and SIPR-alpha.

In aspects of the provided embodiments, a source cell or viral particle producer cell can be engineered to express CD24 or a biologically active portion thereof (or in some cases additionally CD47 or a biologically active portion thereof). Any of a variety of engineering methods are contemplated, including, but not limited to, transient co-transfection, lentiviral integration, use of artificial chromosomes such as bacterial artificial chromosomes (BAC), or gene editing for stable expression at a safe-harbor locus. Expression can be driven by a constitutive or an inducible promoter as desired, such as depending on the particular application or the effect of heterologous expression of CD24 or a biologically active portion thereof (and, in some cases also CD47 or a biologically active portion thereof) on the behavior of the source or producer cells. In other embodiments, CD24 or a biologically active portion thereof (and, in some cases also CD47 or a biologically active portion thereof) can be covalently or non-covalently conjugated to an exposed surface of the non-cell particle.

In a particular embodiment, producer cells used to produce viral vector particles (e.g. lentiviral vector particles) can be engineered to express CD24 or a biologically active portion thereof, or in some cases additionally CD47 or a biologically active portion thereof. Once viral vector particles are produced the CD24 adhesion molecule (and, in some cases CD47 adhesion molecule), or a biologically active fragment of the foregoing, is loaded into the envelope of budding viral particles. The viral particles (e.g. lentiviral vector particles) can additionally be engineered to express an exogenous agent, such as a nucleic acid encoding a therapeutic agent.

Also provided are non-cell particles additionally containing one or more exogenous agent, such as for delivery of a diagnostic or therapeutic agent to cells, including following in vivo administration to a subject. Also provided herein are methods and uses of the non-cell particles, such in diagnostic and therapeutic methods. Also provided are polynucleotides, methods for engineering, preparing, and producing the CD24-associated non-cell particles, compositions containing the particles, and kits and devices containing and for using, producing and administering the particles.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “non-cell particle” refers to any biological or synthetic particle that does not contain a nucleus. Examples of non-cell particles include solid particles such as nanoparticles, viral-derived particles or cell-derived particles. Such non-cell particles include, but are not limited to, viral particles (e.g. lentiviral particles), virus-like particles, dendrimers, exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a non-cell particle can be a fusosome. In some embodiments, the non-cell particle is not a platelet.

As used herein, “biologically active” as used herein relative to CD24 or a portion thereof, means a full length or fragment of CD24, that when exposed on the surface of a non-cell particle, e.g. viral particle, recognizes and binds Siglec-10 in vivo in a human. The CD24 fragment may be any length fragment ranging from 10 to 32 amino acid length chain, and preferably is between about 27 and 32 amino acid length chains, and any and all increments there between. An exemplary biologically active fragment includes the sequence set forth in SEQ ID NO:2. Generally, a biologically active CD24 is a glycoprotein containing linked sialic acids, such as a 2-3-linked sialoside and/or a 2-6-linked sialoside.

The term “biologically active” as used herein relative to CD47 or a portion thereof, means a full length or fragment of CD47, that when exposed on the surface of a non-cell particle, e.g. viral particle, recognizes and binds to SIRPα in vivo in a human. The CD47 fragment may be any length fragment ranging from 10 to 304 amino acid length chains, and preferably is between about 15 and 150 amino acid length chains, and any and all increments there between.

As used herein, a “CD24-associated non-cell particle” refers to non-cell particle in which CD24 or a biologically active portion thereof is exposed on the surface of the non-cell particle. Any of a variety of methods can be used to expose CD24 or a biologically active portion thereof on the surface such as to display, present, express or link (directly or indirectly) CD24 or the biologically active portion on the surface, including recombinant DNA methods or chemical methods, so long as CD24 or the biologically active portion retains the ability to bind Siglec-10 and/or has the biological activity of evading phagocytosis by a phagocytic cell. In some cases, a CD24-associated particle can additionally include CD47 or a biologically active portion thereof that also is exposed on the surface of the non-cell particle. Any of a variety of methods can also be used to expose CD47 or a biologically active portion thereof on the surface such as to display, present, express or link (directly or indirectly) CD47 or the biologically active portion on the surface, including recombinant DNA methods or chemical methods, so long as such particles are those in which the CD24 or the biologically active portion retains the ability to bind Siglec-10 and the CD47 or the biologically active portion thereof retains the ability to bind Sirp-alpha and/or has the biological activity of evading phagocytosis by a phagocytic cell.

As used herein, “lipid particle” refers to any biological or synthetic particle that contains a bilayer of amphipathic lipids enclosing a lumen or cavity. Typically a lipid particle does not contain a nucleus. Examples of lipid particles include solid particles such as nanoparticles, viral-derived particles or cell-derived particles. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet. As used herein, “fusosome” refers to a particle containing a bilayer of amphipathic lipids enclosing a lumen or cavity and a fusogen that interacts with the amphipathic lipid bilayer. In embodiments, the fusosome comprises a nucleic acid. In some embodiments, the fusosome is a membrane enclosed preparation. In some embodiments, the fusosome is derived from a source cell.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “targeted envelope protein” refers to a polypeptide that contains a henipavirus G protein attached to a single domain antibody (sdAb) variable domain, such as a VL or VH only sdAb, nanobodies, camelid VHH domains, shark IgNAR or fragments thereof, that targets a molecule on a desired cell type. In some such embodiments, the attachment may be directly or indirectly via a linker, such as a peptide linker.

As used herein, a “targeted lipid particle” refers to a lipid particle that contains a targeted envelope protein embedded in the lipid bilayer. The terms “decreased” or “reduced” as used herein in the context of one or more activities such as phagocytosis or immunogenicity with respect to a provided CD24-associated non-cell particle means to decrease the one or more activities, as compared to a control, such as a similar non-cell particle that does not contain a surface exposed CD24 or biologically active portion thereof (or, in some cases also does not contain a surface exposed CD24 or biologically active portion thereof). Methods of assessing phagocytosis, immunogenicity or half-life or other relevant activity include assays described in Examples or other assays as described herein. In some embodiments, an activity can be decreased or reduced by at least 5%, 10%, 15%, 20%, 25%, 30%, 35%, 40%, 45%, 50%, 60%, 70%, 80%, 90%, or 100%, as compared to a value of a control.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a non-cell particle, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, “operably linked” or “operably associated” includes reference to a functional linkage of at least two sequences. For example, operably linked includes linkage between a promoter and a second sequence, wherein the promoter sequence initiates and mediates transcription of the DNA sequence corresponding to the second sequence. Operably associated includes linkage between an inducing or repressing element and a promoter, wherein the inducing or repressing element acts as a transcriptional activator of the promoter.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE.

In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, a “target cell” refers to a cell of a type to which it is desired that a non-cell particle delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a non-cell particle delivers an exogenous agent. In some embodiments, a nontarget cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein, “dendrimer” refers to a well-defined, highly branched macromolecule that radiates from a central core and is synthesized in a stepwise, repetitive reaction sequence.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

As used herein, the terms “effective amount” and “pharmaceutically effective amount” refer to a nontoxic but sufficient amount of an agent or drug to provide the desired biological result. That result can be reduction and/or alleviation of the signs, symptoms, or causes of a disease or disorder, imaging or monitoring of an in vitro or in vivo system (including a living organism), or any other desired alteration of a biological system. An appropriate effective amount in any individual case may be determined by one of ordinary skill in the art using routine experimentation.

### II. CD24 ASSOCIATED NON-CELL PARTICLES

Provided herein are non-cell particles that comprise CD24 or a biologically active portion thereof on an exposed surface of the particle. In some embodiments, the non-cell particles comprise CD24 or a biologically active portion thereof and CD47 or a biologically active portion thereof, each on an exposed surface of the particle.

A. CD24

In some embodiments, the non-cell particle includes a CD24 protein or a biologically active portion thereof exposed on the surface of the non-cell particle. The CD24 may be a full-length protein, or it may be a portion of the protein, wherein the portion is biologically active. Typically, the portion of CD24 includes a least a portion of the extracellular domain that interacts with Siglec-10. In some embodiments, the SIGLEC-10 is expressed on immune cell surfaces including B-cells, NK-cells, eosinophils, basophils, thymocytes and T-cells.

In some embodiments, the CD24 or a biologically active portion is a human CD24 or biologically active portion. In some embodiments, the CD24 or biologically active portion comprises the extracellular domain of human CD24 or a binding portion thereof that binds Siglec-10. In some embodiments, the extracellular domain comprises SEQ ID NO: 2 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2. In particular embodiments, any of such sequences bind SIGLEC-10.

In some embodiments, the CD24 or biologically active portion is or contains the extracellular domain set forth in SEQ ID NO: 18 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:18. In particular embodiments, any of such sequences bind SIGLEC-10.

In some embodiments, the CD24 or biologically active portion is between at or about 27 and at or about 32 amino acids in length, or any value between the foregoing. In some aspects, the CD24 is a biologically active portion of CD24 that is longer or shorter, so long as the protein when surface exposed is able to bind to Siglec-10 and/or evade or reduce phagocytosis. In some embodiments, the CD24 is a biologically active portion of CD24 that is an N-terminally and/or C-terminally truncated fragment of the extracellular domain of CD24, such as human CD24. In some embodiments, a biologically active portion of CD24 contains a contiguous sequence of amino acids set forth in SEQ ID NO:2 but that lacks 1, 2, 3, 4, 5 6 or more amino acids on the N-terminus and/or C-terminus of the sequence. In some embodiments, the CD24 is a partial protein or peptide fragment comprising a generic spacer domain fused to the amino acid chain of CD24, so that any required structural features of the partial protein or peptide fragment that contains biological activity are not disrupted.

In some embodiments, the CD24 or biologically active portion thereof is exposed on the surface of the non-cell particle via an attachment moiety. In some cases, the CD24 or biologically active portion may further be modified to include various tagging or labeling of any sort that provides for attachment of the protein to an exposed surface of the particle. In some embodiments, the attachment moiety is a transmembrane domain or other membrane-anchoring functional domain, such that the final recombinant protein effectively anchors into the non-cell particle.

In some embodiments, the attachment moiety is a Glycosylphosphatidylinositol (GPI) membrane tail. In some embodiments, the GPI tail is displaced by a lipid anchor. In some embodiments, prior to displacement by the lipid anchor, the CD24 or the biologically active portion thereof comprises the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3. In some embodiments, displacement by the GPI tail occurs at or between Ala31 and Ala35 of SEQ ID NO:3. In particular embodiments, any of such sequences bind SIGLEC-10.

In some embodiments, the attachment moiety is a transmembrane domain. In some embodiments, the transmembrane domain is on the C-terminal portion of the protein. In some embodiments, the C terminal portion is lipophilic. In some embodiments, the transmembrane domain is a heterologous transmembrane domain.

In some embodiments, the CD24 or biologically active portion thereof is a glycoprotein in which one or more amino acid residues are glycosylated. In some embodiments, the glycosylation is an N-linked glycosylation. In some embodiments, the glycosylation is an O-linked glycosylation. In some embodiments, one or more of 16 potential glycosylation sites in the biologically active portion comprising SEQ ID NO:2 are glycosylated. In some embodiments, one or more of the Ser(S) amino acids are O glycosylated. In some of any embodiments, one or more of the Thr (T) amino acids are O glycosylated. In some of any embodiments, one or more of the Asn (N) amino acids are glycosylated.

In some embodiments, the molecular weight of the protein is between at or about 25 and at or about 30 kDa, at or about 30 and at or about 35 kDa, at or about 35 and at or about 40 kDa, at or about 40 and at or about 45 kDa, at or about 45 and at or about 50 kDa, at or about 50 and at or about 55 kDa, at or about 55 and at or about 60 kDa, at or about 60 and at or about 65 kDa, or at or about 65 and at or about 70 kDa, each inclusive. In some embodiments, the molecular weight of the protein is about 25 kDa, about 30 kDa, about 40 kDa, about 45 kDa, about 50 kDa, about 55 kDa, about 60 kDa, about 65 kDa or about 70 KDa.

In some embodiments, the one or more glycans is a sialylated glycan. In some embodiments, the CD24 or biologically active portion thereof binds SIGLEC-10 via sialylated glycans. In some embodiments, the CD24 or biologically active portion contains α2,3 linked sialic acids. In some embodiments, the CD24 or biologically active portion contains α2-6 linked sialic acids. In some embodiments, the CD24 or biologically active portion contains α2,3 linked sialic acids and α2-6 linked sialic acids.

In some embodiments, the CD24 can be resialylated. In some embodiments, resialylation comprises the use of sialyltransferases. In some embodiments CD24 is re-sialylated to an α2-3- or α2-6-sialyl linkage.

In some embodiments, methods for producing non-cell particles can include expression of CD24 in cells that are capable of glycosylation. In some embodiments, cells include human cell lines. In some embodiments, human cell lines comprise HEK293, PER.C6, CEVEC's amniocyte production (CAP), AGE1.HN, HKB-11 and HT-1080 cells. In some embodiments, the cell lines are non-human mammalian cell lines. In some embodiments, the cell lines comprise Chinese hamster ovary (CHO) cells, COS-1 non-human primate cells, baby hamster kidney (BHK) cells, NSO myeloma and Sp2/0 hybridoma mouse cell lines, human embryonic kidney cells 293 (HEK293) and HT-1080 human cells.

In some embodiments, the CD24 or a biologically active portion thereof is encoded by a nucleotide sequence that encodes an amino acid sequence containing the sequence set forth in SEQ ID NO: 1. In some embodiments, the CD24 or a biologically active portion thereof is encoded by a nucleotide sequence that encodes an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:1. In particular embodiments, any of such sequences bind SIGLEC-10.

In some embodiments, the non-cell particle includes CD24 or a biologically active portion that encoded by a polynucleotide comprising SEQ ID NO:4. In some embodiments, the CD24 or a biologically active portion thereof is encoded by a nucleotide sequence that has at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:4. In particular embodiments, any of such sequences bind SIGLEC-10.

In some embodiments, the CD24 or a biologically active portion thereof is encoded by a nucleotide sequence that encodes an amino acid sequence containing the sequence set forth in SEQ ID NO: 15. In some embodiments, the CD24 or a biologically active portion thereof is encoded by a nucleotide sequence that encodes an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:15. In particular embodiments, any of such sequences bind SIGLEC-10.

In some embodiments, the CD24 or a biologically active portion thereof is encoded by a nucleotide sequence that encodes an amino acid sequence containing the sequence set forth in SEQ ID NO: 16. In some embodiments, the CD24 or a biologically active portion thereof is encoded by a nucleotide sequence that encodes an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:16. In particular embodiments, any of such sequences bind SIGLEC-10.

In some embodiments, the CD24 or a biologically active portion thereof is encoded by a nucleotide sequence that encodes an amino acid sequence containing the sequence set forth in SEQ ID NO: 17. In some embodiments, the CD24 or a biologically active portion thereof is encoded by a nucleotide sequence that encodes an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:17. In particular embodiments, any of such sequences bind SIGLEC-10.

In some embodiments, the polynucleotide encoding the CD24 or a biologically active portion is under the control of a promoter or a heterologous expression control element. In some embodiments, the promoter is inducible. In some embodiments, the promoter is constitutive.

In some embodiments, the CD24 or biologically active portion is present on the exposed surface of the non-cell particle at a sufficient concentration so as to effectively be recognized by Siglec-10. This concentration can be measured as a ratio of the number of surface marker (molecules) per m². In some embodiments, the amount of the biologically active CD24 or portion thereof on the non-cell particle is about 10 molecules/μ², about 20 molecules/μ², about 50 molecules/μ², about 75 molecules/μ², about 100 molecules/μ², about 100 molecules/μ², about 125 molecules/μ², about 150 molecules/μ², about 175 molecules/μ², about 200 molecules/μ², about 225 molecules/μ², or about than 250 molecules/μ². In some embodiments, the amount of the biologically active CD24 or portion thereof on the non-cell particle is between about 20 and 250 molecules/μ², between about 20 and 50 molecules/μ², between about 50 and 75 molecules/μ², between about 75 and 100 molecules/μ², between about 100 and 125 molecules/μ², between about 125 and 150 molecules/μ², between about 150 and 175 molecules/μ², between about 175 and 200 molecules/μ², between about 200 and 225 molecules/μ², between about 225 and 250 molecules/μ². In some embodiments, the mean amount of the biologically active CD24 or portion thereof on a population of non-cell particles is about 10 molecules/μ², about 20 molecules/μ², about 50 molecules/μ², about 75 molecules/μ², about 100 molecules/μ², about 100 molecules/μ², about 125 molecules/μ², about 150 molecules/μ², about 175 molecules/μ², about 200 molecules/μ², about 225 molecules/μ², or about than 250 molecules/μ². In some embodiments, the mean amount of the biologically active CD24 or portion thereof on a population of non-cell particles is between about 20 and 250 molecules/μ², between about 20 and 50 molecules/μ², between about 50 and 75 molecules/μ², between about 75 and 100 molecules/μ², between about 100 and 125 molecules/μ², between about 125 and 150 molecules/μ², between about 150 and 175 molecules/μ², between about 175 and 200 molecules/μ², between about 200 and 225 molecules/μ², between about 225 and 250 molecules/μ².

B. CD47

In some embodiments, the non-cell particle also includes a CD47 protein or a biologically active portion thereof exposed on the surface of the non-cell particle. The CD47 may be a full-length protein, or it may be a portion of the protein, wherein the portion is biologically active. In some embodiments, the portion of CD47 includes at least a portion of the extracellular domain that interacts with SIRPα(SIRP-alpha). SIRP-alpha is highly polymorphic within both human and mouse (Strowing, et al., 2011, Proc. Natl. Acad. Sci. USA. 108(32):13218-23), with the likely consequence that this minimizes pathogen interactions with SIRP-alpha (Hatherley et al., 2008, Mol Cell 31(2):266-77).

In some embodiments, the CD47 or biologically active portion thereof is a human CD47 or biologically active portion.

In some embodiments, the CD47 or biologically active portion comprises the extracellular domain of human CD47 or a binding portion thereof that binds SIRPα. In some embodiments, the extracellular domain comprises SEQ ID NO: 7. In some embodiments, the extracellular domain comprises an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7. In some embodiments, the extracellular domain of the CD47 is encoded by a sequence of nucleotides encoding the amino acid sequence set forth in SEQ ID NO: 6 an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:6. In particular embodiments, any of such sequences bind SIRP-alpha.

In some embodiments, the CD47 or biologically active portion is between at or about 10 and at or about 304 amino acids in length, or any value between the foregoing. In some embodiments, the CD47 or biologically active portion is between at or about 10 and at or about 150 amino acids in length, or any value between the foregoing. In some aspects, the CD47 is a biologically active portion of CD47 that is longer or shorter, so long as the protein when surface exposed is able to bind to SIRP-alpha and/or evade or reduce phagocytosis. In some embodiments, the CD47 is a biologically active portion of CD47 that is an N-terminally and/or C-terminally truncated fragment of the extracellular domain of CD47, such as human CD47. In some embodiments, a biologically active portion of CD47 contains a contiguous sequence of amino acids set forth in SEQ ID NO:7 but that lacks 1, 2, 3, 4, 5, 6, 7, 8, 9, 10, 20, 30, 40, 50, 60, 70, 80, 90, 100, 110 or more amino acids on the N-terminus and/or C-terminus of the sequence. Residues of CD47 involved in binding SIRP-alpha are known (see e.g. Hatherley et al. 2008 Mol. Cell, 31:266-277). In some embodiments, the biologically active portion contains amino acid residues EVTELTREGE (SEQ ID NO:11). In some embodiments, the CD47 is a partial protein or peptide fragment comprising a generic spacer domain fused to the amino acid chain of CD47, so that any required structural features of the partial protein or peptide fragment that contains biological activity are not disrupted.

In some embodiments, the portion of the CD47 comprises a 10-aa FG-hairpin comprising the sequence EVTELTREGE (SEQ ID NO:11) centered on the interacting loop. In some embodiments, a biologically active portion of the CD47 has the sequence GNYTCEVTELTREGETIIELK (SEQ ID NO: 9). In some embodiments, a biologically active portion of the CD47 comprises SEQ ID NO: 10. In some embodiments, when both occurrences of Xaa are C of SEQ ID NO:10, the two cysteine residues may exist in a cyclic disulfide-linked form (i.e., form a cyclic intramolecular cysteine form). Both the non-cyclic cysteine form of peptide of SEQ ID NO;10 and the cyclic cystine form of SEQ ID NO:10 are contemplated. In some embodiments, a biologically active portion of CD47 has a 12-aa FG-SS-peptide comprising the sequence CEVTELTREGEC (SEQ ID NO:12) with a Cys substitution opposite in the hairpin to Cys intended to disulfide-stabilize the P-hairpin. In some embodiments, the biologically active portion of the CD47 sequence binds SIRP-alpha.

In some embodiments, the CD47 or biologically active portion thereof is exposed on the surface of the non-cell particle via an attachment moiety. In some cases, the CD47 or biologically active portion may further be modified to include various tagging or labeling of any sort that provides for attachment of the protein to an exposed surface of the particle. In some embodiments, the attachment moiety is a transmembrane domain or other membrane-anchoring functional domain, such that the final recombinant protein effectively anchors into the non-cell particle.

In some embodiments, the attachment moiety is a transmembrane domain. In some embodiments, the transmembrane domain is on the C-terminal portion of the protein. In some embodiments, the C terminal portion is lipophilic. In some embodiments, the transmembrane domain is a heterologous transmembrane domain. In some embodiments, the transmembrane domain is a transmembrane domain of CD47, such as human CD47. In some embodiments, the transmembrane domain contains the sequence of amino acids set forth in SEQ ID NO:14, or a sequence of amino acids having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14. In particular embodiments, any of such sequences bind SIRP-alpha.

In some embodiments, the CD47 or a biologically active portion thereof contains the sequence set forth in SEQ ID NO: 8. In some embodiments, the CD47 or biologically active portion comprises an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8. In some embodiments, the CD47 or a biologically active portion thereof is encoded by a nucleotide sequence that encodes an amino acid sequence containing the sequence set forth in SEQ ID NO: 5. In some embodiments, the CD47 or a biologically active portion thereof is encoded by a nucleotide sequence that encodes an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:5. In particular embodiments, any of such sequences bind SIRP-alpha.

In some embodiments, the non-cell particle includes CD47 encoded by a nucleotide sequence comprising SEQ ID NO: 13. In some embodiments, the CD47 or a biologically active portion thereof is encoded by a nucleotide sequence that has at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:13. In particular embodiments, any of such sequences bind SIRP-alpha.

In some embodiments, the polynucleotide encoding the CD47 or a biologically active portion is under the control of a promoter or a heterologous expression control element. In some embodiments, the promoter is inducible. In some embodiments, the promoter is constitutive.

In some embodiments, the non-cell particle contains a polynucleotide that contains a first nucleic acid sequence encoding CD24 or a biologically active portion, such as any described, and a second nucleic acid encoding CD47 or a biologically active portion, such as any described. In some embodiments, the first and second nucleic acids are operatively linked to the same promoter or heterologous expression control element. In some embodiments, the polynucleotide comprises a sequence encoding a linking peptide between the first and second nucleic acid sequences, wherein the linking peptide separates the translation products of the first and second nucleic acid sequences during or after translation. In some embodiments, the linking peptide comprises an internal ribosome entry site (IRES), a self-cleaving peptide, or a peptide that causes ribosome skipping, optionally a T2A peptide.

In another embodiment, the amount of the biologically active CD47 or portion thereof on the non-cell particle is about 10 molecules/μ², about 20 molecules/μ², about 50 molecules/μ², about 75 molecules/μ², about 100 molecules/μ², about 100 molecules/μ², about 125 molecules/μ², about 150 molecules/μ², about 175 molecules/μ², about 200 molecules/μ², about 225 molecules/μ², or about than 250 molecules/μ². In some embodiments, the amount of the biologically active CD24 or portion thereof on the non-cell particle is between about 20 and 250 molecules/μ² , between about 20 and 50 molecules/μ², between about 50 and 75 molecules/μ², between about 75 and 100 molecules/μ², between about 100 and 125 molecules/μ², between about 125 and 150 molecules/μ², between about 150 and 175 molecules/μ², between about 175 and 200 molecules/μ², between about 200 and 225 molecules/μ², between about 225 and 250 molecules/μ². In another embodiment, the mean amount of the biologically active CD47 or portion thereof on a population of non-cell particles is about 10 molecules/μ², about 20 molecules/μ², about 50 molecules/μ², about 75 molecules/μ², about 100 molecules/μ², about 100 molecules/μ², about 125 molecules/μ², about 150 molecules/μ², about 175 molecules/μ², about 200 molecules/μ² about 225 molecules/μ², or about than 250 molecules/μ². In some embodiments, the mean amount of the biologically active CD47 or portion thereof on a population of non-cell particles is between about 20 and 250 molecules/μ², between about 20 and 50 molecules/μ², between about 50 and 75 molecules/μ², between about 75 and 100 molecules/μ², between about 100 and 125 molecules/μ², between about 125 and 150 molecules/μ², between about 150 and 175 molecules/μ², between about 175 and 200 molecules/μ², between about 200 and 225 molecules/μ², between about 225 and 250 molecules/μ².

C. Polynucleotides Encoding CD24 and/or CD47

Provided herein is a polynucleotide comprising a first nucleic acid sequence encoding CD24 or a biologically active portion thereof. In some embodiments, the polynucleotide comprises a second nucleic acid encoding CD47 or a biologically active portion thereof. The polynucleotides can be used for expressing the CD24 or biologically active portion alone or with a CD47 or a biologically active portion thereof in a producer cell or source cell in connection with preparing any of the particles provided herein.

In some embodiments, the polynucleotide contains at least one promoter that is operatively linked to control expression of the CD24 or the CD47. In some embodiments, promoter elements regulate the frequency of transcriptional initiation. In some examples, each of the nucleic acids encoding CD24 and CD47 (e.g. first and second nucleic acid) of the provided polynucleotides are operatively linked to a promoter. In some embodiments, the promoters are the same. In some embodiments, the promoters are different. For expression of the surface molecule, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation. In some of any embodiments, expression of natural or synthetic nucleic acids is typically achieved by operably linking a nucleic acid encoding the gene of interest to a promoter and incorporating the construct into an expression vector. In some embodiments, vectors can be suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, a nucleic acid encoding CD24 and/or CD47 may be contained in an expression vector. Vectors comprising nucleic acids that encode a CD24 and/or CD47 or polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In particular, a DNA vector that encodes a CD24 and/or CD47 can be used to facilitate the the expression and recombinant production of a CD24 and/or CD47. The DNA sequence can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used. The methods producing a a CD24 and/or CD47 polypeptide may include culturing a cell under conditions that lead to expression of the polypeptide, wherein the cell comprises a nucleic acid molecule encoding a CD24 and/or CD47 described herein, and/or vectors that include these nucleic acid sequences. In some embodiments, a polynucleotide expressed in prokaryotic cells, such as bacterial cells; or in eukaryotic cells, such as fungal cells (such as yeast), plant cells, insect cells, and mammalian cells. Exemplary non-cell particles for delivery are described in Section III.

In the production of viral particles, a second vector encoding any required capsid, envelope and/or matrix proteins is introduced as a transient transfection of the cell line to provide for effective viral particle formation. During construction of the virion, the expressed surface marker may be incorporated into the virion along with the other viral proteins.

In some embodiments, additional promoter elements, e.g., enhancers, regulate the frequency of transcriptional initiation. In some embodiments, additional promoter elements are located in the region 30-110 bp upstream of the start site, although a number of promoters have recently been shown to contain functional elements downstream of the start site as well. In some embodiments, spacing between promoter elements frequently is flexible, so that promoter function is preserved when elements are inverted or moved relative to one another. In some embodiments, the thymidine kinase (tk) promoter, the spacing between promoter elements can be increased to 50 bp apart before activity begins to decline. In some embodiments, depending on the promoter, individual elements can function either cooperatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-1a (EF-1 a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is a constitutive promoter. In some aspects, a constitutive promoters may be a ubiquitous promoter that allows expression in a wide variety of cell and tissue types. In some embodiments, the promoter is a human Ubiquitin C (UbC) promoter, a human elongation factor 1α (EF1α) promoter, an SV40 promoter, a Cytomegalovirus (CMV) promoter, a CAG promoter, or a PGK-1 promoter.

In some embodiments, the promoter is an inducible promoter. In some embodiments, the inducible promoter provides a molecular switch capable of turning on expression of the polynucleotide sequence which it is operatively linked when such expression is desired, or turning off the expression when expression is not desired. In some embodiments, inducible promoters comprise metallothionine promoter, a glucocorticoid promoter, a progesterone promoter, and a tetracycline promoter.

In some embodiments, exogenously controlled inducible promoters can be used to regulate expression of the CD24 or the CD47. For example, radiation-inducible promoters, heat-inducible promoters, and/or drug-inducible promoters can be used to selectively drive transgene expression in, for example, targeted regions. In such embodiments, the location, duration, and level of transgene expression can be regulated by the administration of the exogenous source of induction.

In some embodiments, expression of the CD24 or the CD47 is regulated using or a drug-inducible promoter. For example, in some cases, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, can be combined with a selected promoter to enhance transcription in the presence of drug, such as doxycycline. In some embodiments, the drug-inducible promoter is a small molecule-inducible promoter.

In some embodiments, such as those where the polynucleotide contains a first and second nucleic acid sequence, the polynucleotide further contains a nucleic acid sequence encoding a linking peptide between the first and second nucleic acid sequences. In some such cases, the linking peptide separates the translation products of the first and second nucleic acid sequences during or after translation. In some aspects, the linking peptide contains an internal ribosome entry site (IRES), a self-cleaving peptide, or a peptide that causes ribosome skipping, such as a T2A peptide.

In some embodiments, the vector or construct can contain a single promoter that drives the expression of one or more nucleic acid molecules. In some embodiments, such promoters can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g. encoding a first and second recombinant receptor) by a message from a single promoter. Alternatively, in some cases, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein, without limitation, 2A sequences from the foot-and-mouth disease virus (F2A), equine rhinitis A virus (E2A), Thosea asigna virus (T2A), and porcine teschovirus-1 (P2A) as described in U.S. Patent Publication No. 20070116690.

Any of the provided polynucleotides can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized.

Methods of codon optimization and CpG motif detection and modification are well-known.

Typically, polynucleotide optimization enhances transgene expression, increases transgene stability and preserves the amino acid sequence of the encoded polypeptide.

In order to assess the expression of the surface molecule, the expression vector to be introduced into a cell can also contain either a selectable marker gene or a reporter gene or both to facilitate identification and selection of expressing particles, e.g. viral particles. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into any desired host cell for expression of CD24 and/or CD24 host cell. The cell can be any source cell or producer cell for producing the non-cell particles described herein. In some embodiments, the host cell is a mammalian, bacterial, yeast or insect cell. Any of various methods for introducing the expression vector can be used, such as by physical, chemical or biological means. Such methods include, but are not limited to transient co-transfection, lentiviral integration, BAC or by gene-editing for stable integration into a safe-harbor locus.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2001, Molecular Cloning: A Laboratory Manual, Cold Spring Harbor Laboratory, New York, and in Ausubel et al., 1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Other viral vectors can be derived from lentivirus, poxviruses, herpes simplex virus I, adenoviruses and adeno-associated viruses, and the like. See, for example, U.S. Pat. Nos. 5,350,674 and 5,585,362.Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. In some embodiments, a colloidal system for use as a delivery vehicle is a liposome (i.e., an artificial membrane vesicle).

Non-viral vectors and other chemical means employing the use of polymers, surfactants, and/or excipients have been employed to introduce polynucleotides and polypeptides into cells including conjugation with a targeting moiety, conjugation with a cell penetrating peptide, derivatization with a lipid and incorporation into liposomes, lipid nanoparticles, and cationic liposomes. The majority of non-viral vectors consist of plasmid DNA complexed with lipids or polycations. Many different lipids with ability to deliver plasmid DNA to cells in vitro and in vivo have been reported (Gao, et al., Gene Therapy 2:710-722 (1995)).

In some embodiments, the polynucleotide is delivered as a naked nucleic acid. In some embodiments, the polynucleotide is administered as an mRNA. In some embodiments, the polynucleotide is administered as DNA, e.g., a plasmid.

Polynucleotides encoding CD24 and/or CD47 of the present invention may be delivered to a cell naked. As used herein in, “naked” refers to delivering a polynucleotide free from agents which promote internalization. For example, the polynucleotide delivered to the cell may contain no modifications. The naked polynucleotides may be delivered to the cell using routes of administration known in the art and described herein.

In some aspects, mRNAs may be delivered as packaged particles (e.g., encapsulated in a delivery vehicle) or unpackaged (i.e., naked). In some aspects, mRNA may be transcribed within host cells. Exogenous mRNA delivery was first investigated in 1990, wherein Wolff and colleagues observed protein expression in mice following injection of mRNA encoding a reporter gene (Wolff et al., Science (247) 1465, 1990). Once exogenous mRNA has been transmitted to the cytosol, in some aspects, host cellular machinery can produce a mature polypeptide. In some embodiments, the polypeptide can be subject to post-translational modifications. In some aspects, proteins produced from exogenous mRNA delivery are degraded by normal physiological processes. In some embodiments, mRNA delivery reduces risk of metabolite toxicity (Pardi et al., Nat Rev Drug Discov (17) 4, 2018).

According to the provided embodiments, polynucleotides administered mRNA may have a capping region. The capping region may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

Wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present invention to, among other things, enhance the stability of the molecule. In some aspects, the in vivo half-life of mRNA can be regulated via modifications to the 3′ poly-adenosine tail. The specific features can also be incorporated to ensure controlled down-regulation of the transcript in case they are misdirected to undesired organs sites.

D. Chemical or Non-Chemical Linkage

Among provided non-cell particles include particles in which CD24 or a biologically active portion (or in some cases also CD47 or a biologically active portion) is covalently or non-covalently bound to the surface of the non-cell particle. The linkage between the non-cell particles disclosed herein may be linked directly or indirectly, i.e. CD24, CD47, and/or a biologically active portion thereof directly adjoin or they may be linked by an additional component of the complex, e.g. a linker.

In some embodiments, the linkage can be via hydrogen bonds or via complexation (complex bond) or via intercalation or via lipophilic interactions by means of a linker.

In some embodiments, a non-cell particle, such as a nanoparticle, is functionalized for attachment of a CD24 or biologically active portion (or in some cases also CD47 or a biologically active portion). In some embodiments, a non-cell particle is functionalized to contain a plurality of functional groups for attachment of CD24 or biologically active portion (or in some cases also CD47 or a biologically active portion). In particular aspects, attachment to the CD24 or biologically active portion (or in some cases also the CD47 or biologically active portion) is via a moiety immobilized or affixed on the surface of the particle, e.g. nanoparticle. In some cases, the CD24 or biologically active portion (or in some cases also the CD47 or the biologically active portion) may contain a linker for attachment to the moiety present on the surface of the particle. In some embodiments, the linker for attachment of the CD24 or the biologically active portion is different from the linker for attachment of the CD47 or the biologically active portion.

The moiety may be derivatized to contain functional groups at their distal ends.

These functional groups may be useful in allowing the moiety to bind to the CD24 and/or CD47 or biologically portions thereof, such as via a linker. In some embodiments, the moiety can be a member of a binding pair such as for example, Glutathione-S-transferase/glutathione, 6× Histidine Tag/Ni-NTA, Streptavidin/biotin, S-protein/S-peptide, Cutinase/phosphonate inhibitor, antigen/antibody, hapten/anti-hapten, folic acid/folate binding protein, and protein A or G/immunoglobulins. In such embodiments, the linker can be the other member of the binding pair. In some embodiments, the binding pair involves an enzyme-substrate or ligand-receptor interactions, which can be used as a stable mode of linkage between molecules and particle. For example, biotin and its binding partners, e.g., avidin, streptavidin, NeutrAvidin, CaptAvidin, etc., form a strong and stable complex when they interact. In such an example, the binding partners are not covalently bonded, yet the association is extremely stable, with a dissociation constant of about 1.3×10˜15 M. In some embodiments, attachment via CD24 or a biologically active portion or CD47 and a biologically active portion are carried out using different binding partner pairs.

In some embodiments, CD24 or a biologically active portion (or in some cases CD47 or a biologically active portion) can be modified with biotin, or biotinylated, and coupled to streptavidin or avidin molecules immobilized on the particle surfaces via chemical bonding. In other embodiments, the CD24 or a biologically active portion (or in some cases CD47 or a biologically active portion) can be linked to strepavidin, and coupled to the biotinylated surfaces of the particle. Any of a variety of particles as described can be modified or derivatized with such moieties. For example, any of a variety of lipid-based particles, such as liposomes, micelles, or viral-based particles, can be prepared so that the composition contains biotinylated lipids, or lipids modified with avidin, streptavidin, or NeutrAvidin. In such cases, the lipid-based non-cell particles, such as viral and cell derived particles, become targeted as the binding will only occur between specific partners.

In some embodiments, the particle is at least partially coated with streptavidin and the the CD24 or a biologically active portion (or in some cases CD47 or a biologically active portion) is biotinylated.

In some embodiments, to functionalize particles, a recombinant protein, or portion of a protein that is biologically active, can be constructed and attached to the particles using recombinant DNA methodology, such as, for example, that described in Sambrook and Russell (2001, Molecular Cloning, A Laboratory Approach, Cold Spring Harbor Press, Cold Spring Harbor, N.Y.), and Ausubel et al. (2002, Current Protocols in Molecular Biology, John Wiley & Sons, NY). In some cases, such procedures can include the introduction of amino acid changes in a protein or polypeptide by altering the DNA sequence encoding the polypeptide.

In a particular example, plasmid encoding CD24 or a biologically active portion, a spacer region and a biotin site can each be amplified and ligated into a vector, which will result in an in-frame fusion of a spacer peptide-biotin at the c-terminus of the CD24 or the biologically active portion thereof. The vector can then be transfected into cells and the resulting recombinant protein can be secreted and biotinylated at the C-terminus. After coating the particles with streptavidin, the particles can be incubated with the biotinylated recombinant protein, such that the recombinant protein adheres to an exposed surface of the particle. In some embodiments, CD47 or a biologically active portion thereof can be similarly attached using the same or a different binding partner pair.

### III. TYPES OF NON-CELL PARTICLES

Among the provided CD24-associated non-cell particles are non-cell particles comprising a biological or synthetic particle that do not contain a nucleus. In some embodiments, the non-cell particle comprises a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell derived particle.

In particular embodiments, the non-cell particle is virally derived. In some embodiments, the lipid particle can be a viral particle, a virus-like particle, a lentivirus, a viral vector, or a lentiviral vector, a viral based particle, or a virus like particle (VLP).

In particular embodiments, the non-cell particle is not virally derived. In some embodiments, the lipid particle can be a a nanoparticle, a vesicle, an exosome, a dendrimer, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

Among provided CD24-associated non-cell particles are fusosomes. In some embodiments, the fusosome comprises a naturally derived bilayer of amphipathic lipids with a fusogen. In some embodiments, the fusosome comprises (a) a lipid bilayer, (b) a lumen (e.g., comprising cytosol) surrounded by the lipid bilayer; and (c) a fusogen that is exogenous or overexpressed relative to the source cell. In some embodiments, the fusogen is disposed in the lipid bilayer. In some embodiments, the fusosome comprises several different types of lipids, e.g., amphipathic lipids, such as phospholipids. In some embodiments, the fusosome comprises a lipid bilayer as the outermost surface. In some embodiments, the bilayer may be comprised of one or more lipids of the same or different type. In some embodiments, the lipids comprise phospholipids such as phosphocholines and phosphoinositols. In some embodiments, the lipids comprise DMPC, DOPC, and DSPC.

In some embodiments, the fusosome is any of the described non-cell particles provided herein into which a fusogen is disposed in the lipid bilayer. In some embodiments, the non-cell particle is a fusosome which comprises one or more fusogens that may mediate membrane fusion of the non-cell particle at a cell surface or in an endosome or in another cell-membrane bound space. Exemplary fusogens, including fusogens for specific targeting of a target cells, are described in Section IV.

The non-cell particles can include spherical particles or can include particles of elongated or irregular shape.

In some embodiments, a composition of particles can be assessed for one or more features related to their size, including diameter, range of variation thereof above and below an average (mean) or median value of the diameter, coeeficient of variation, polydispersity index or other measure of size of particles in a composition. Various methods for particle characterization can be used, including, but not limited to, laser diffraction, dynamic light scattering (DLS; also known as photon correlation spectroscopy) or image analysis, such as microscopy or automated image analysis.

In some embodiments, the non-cell particle has a diameter of, or the average (mean) diameter of particles in a composition is, less than about 3 m, less than about 2 m, less than about 1 m, less than about 900 nm, less than about 800 nm, less than about 700 nm, less than about 600 nm, less than about 500 m, less than about 400 nm, less than about 300, less than about 200 nm, less than about 150 nm, less than about 100 nm, less than about 50 nm, or less than about 20 nm. In some embodiments, the non-cell particle has a diameter of, or the average (mean) diameter of particles in a composition is, less than about 400 nm. In another embodiment, the non-cell particle has a diameter of, or the average (mean) diameter of particles in a composition is, less than about 150 nm. In some embodiments, the non-cell particle has a diameter of, or the average (mean) diameter of particles in a composition is, between at or about 2 m and at or about 1 m, between at or about 1 m and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments the median particle diameter in a composition of particles is between at or about 10 nm and at or about 1000 nM, between at or about 25 nm and at or about 500 nm, between at or about 40 nm and at or about 300 nm, between at or about 50 nm and at or about 250 nm, between at or about 60 nm and at or about 225 nm, between at or about 70 nm and at or about 200 nm, between at or about 80 nm and at or about 175 nm, or between at or about 90 nm and at or about 150 nm.

In some embodiments, 90% of the non-cell particles in a composition fall within 50% of the median diameter of the non-cell particles. In some embodiments, 90% of the non-cell particles in a composition fall within 25% of the median diameter of the non-cell particles. In some embodiments, 90% of the non-cell particles in a composition fall within 20% of the median diameter. In some embodiments, 90% of the non-cell particles in a composition fall within 15% of the median diameter of non-cell particles. In some embodiments, 90% of the non-cell particles in a composition fall within 10% of the median diameter of the non-cell particles.

In some embodiments, 75% of the non-cell particles in a composition fall within +/−2 or +/−1 St Dev standard deviations (St Dev) of the mean diameter of non-cell particles. In some embodiments, 80% of the non-cell particles in a composition fall within +/−2 St Dev or +/−1 St Dev of the mean diameter of non-cell particles. In some embodiments, 85% of the non-cell particles in a composition fall within +/−2 St Dev or +/−1 St Dev of the mean diameter of non-cell particles. In some embodiments, 90% of the non-cell particles in a composition fall within +/−2 St Dev or +/−1 St Dev of the mean diameter of non-cell particles. In some embodiments, 95% of the non-cell particles in a composition fall within +/−2 St Dev or +/−1 St Dev of the mean diameter of non-cell particles.

In some embodiments, the non-cell particles have an average hydrodynamic radius, e.g. as determined by DLS, of about 100 nm to about two microns. In some embodiments, the non-cell particles have an average hydrodynamic radius between at or about 2 m and at or about 1 m, between at or about 1 m and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the non-cell particles have an average geometric radius, e.g. as determined by a multi-angle light scattering, of about 100 nm to about two microns. In some embodiments, the non-cell particles have an average geometric radius between at or about 2 m and at or about 1 m, between at or about 1 m and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the coefficient of variation (COV) (i.e. standard deviation divided by the mean) of a composition of non-cell particles is less than at or about 30%, less than at or about 25%, less than at or about 20%, less than at or about 15%, less than at or about 10% or less than at or about 5%.

In some embodiment, provided compositions of non-cell particles are characterized by their polydispersity index, which is a measure of the size distribution of the particles wherein values between 1 (maximum dispersion) and 0 (identical size of all of the particles) are possible. In some embodiments, compositions of non-cell particles provided herein have a polydispersity index of between at or about 0.05 and at or about 0.7, between at or about 0.05 and at or about 0.6, between at or about 0.05 and at or about 0.5, between at or about 0.05 and at or about 0.4, between at or about 0.05 and at or about 0.3, between at or about 0.05 and at or about 0.2, between at or about 0.05 and at or about 0.1, between at or about 0.1 and at or about 0.7, between at or about 0.1 and at or about 0.6, between at or about 0.1 and at or about 0.5, between at or about 0.1 and at or about 0.4, between at or about 0.1 and at or about 0.3, between at or about 0.1 and at or about 0.2, between at or about 0.2 and at or about 0.7, between at or about 0.2 and at or about 0.6, between at or about 0.2 and at or about 0.5, between at or about 0.2 and at or about 0.4 between at or about 0.2 and at or about 0.3, between at or about 0.3 and at or about 0.7, between at or about 0.3 and at or about 0.6, between at or about 0.3 and at or about 0.5, between at or about 0.3 and at or about 0.4, between at or about 0.4 and at or about 0.7, between at or about 0.4 and at or about 0.6, between at or about 0.4 and at or about 0.5, between at or about 0.5 and at or about 0.7, between at or about 0.5 and at or about 0.6, or between at or about 0.6 and at or about 0.7. In some embodiments, the polydispersity index is less than at or about 0.05, less than at or about 0.1, less than at or about 0.15, less than at or about 0.2, less than at or about 0.25, less than at or about 0.3, less than at or about 0.4, less than at or about 0.5, less than at or about 0.6 or less than at or about 0.7. Various non-cell particles are known, any of which can be generated to incorporate on their exposed surface a CD24 or a biologically active portion thereof, alone or with a CD47 or a biologically active portion thereof, in accord with the provided embodiments. Non-limiting examples of non-cell particles include any as described in, or contain features as described in, International published PCT Application No. WO 2017/095946; WO 2017/095944; WO 2017/095940; WO 2019/157319; WO 2018/208728; WO 2019/113512; WO 2019/161281; WO 2020/102578; WO 2019/222403; WO 2020/014209; WO 2020/102485; WO 2020/102499; WO 2020/102503; WO 2013/148327; WO 2017/182585; WO 2011/058052; or WO 2017/068077, each of which are incorporated by reference in their entirety.

Features of the provided CD24-associated non-cell particles are described in the following subsections.

A. Viral-based Particles

Provided herein are non-cell particles that are derived from virus, such as viral particles or virus-like particles, including those derived from retroviruses or lentiviruses. In some embodiments, the non-cell particle's bilayer of amphipathic lipids is or comprises the viral envelope. In some embodiments, the viral envelope may comprise a fusogen, e.g., a fusogen that is endogenous to the virus or a pseudotyped fusogen. In some embodiments, the non-cell particle's lumen or cavity comprises a viral nucleic acid, e.g., a retroviral nucleic acid, e.g., a lentiviral nucleic acid. In some embodiments, the viral nucleic acid may be a viral genome. In some embodiments, the fusosome further comprises one or more viral non-structural proteins, e.g., in its cavity or lumen. In some embodiments, the non-cell particle is or comprises a virus-like particle (VLP). In some embodiments, the VLP does not comprise an envelope. In some embodiments, the VLP comprises an envelope.

Biological methods for introducing an exogenous agent to a host cell include the use of DNA and RNA vectors. DNA and RNA vectors can also be used to house and deliver polynucleotides and polypeptides. Viral vectors and virus like particles, and especially retroviral vectors, have become the most widely used method for inserting genes into mammalian, e.g., human cells. Other viral vectors and virus like particles can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the viral particles or virus-like particles bilayer of amphipathic lipids is or comprises lipids derived from an infected host cell. In some embodiments, the lipid bilayer is a viral envelope. In some embodiments, the viral particles or virus-like particles envelope is obtained from a host cell. In some embodiments, the viral particles or virus-like particles envelope is obtained by the viral capsid from the source cell plasma membrane. In some embodiments, the lipid bilayer is obtained from a membrane other than the plasma membrane of a host cell. In some embodiments, the viral particles or virus-like particles envelope lipid bilayer is embedded with viral proteins, including viral glycoproteins.

In some embodiments, 90% of the viral particles or virus-like particles, e.g. retroviral particles or retroviral-like particles, fall within 50% of the median diameter of the particle. In some embodiments, 90% of the viral particles or virus-like particles, e.g. retroviral particles or retroviral-like particles, fall within 25% of the median diameter of the particle. In some embodiments, 90% of the viral particles or virus-like particles, e.g. retroviral particles or retroviral-like particles, fall within 20% of the median diameter of the particles. In some embodiments, 90% of the viral particles or virus-like particles, e.g. retroviral particles or retroviral-like particles, fall within 15% of the median diameter of the viral particles. In some embodiments, 90% of the viral particles or virus-like particles, e.g. retroviral particles or retroviral-like particles, fall within 10% of the median diameter of the particles.

In some embodiments, one or more transducing units of viral particles or virus-like particles, e.g. retroviral particles or retroviral-like particles, are administered to the subject. In some embodiments, at least 1, 10, 100, 1000, 10⁴, 10⁵, 10⁶, 10⁷, 10⁸, 10⁹, 10¹⁰, 10¹¹, 10¹², 10¹³, or 10¹⁴, transducing units per kg are administered to the subject. In some embodiments at least 1, 10, 100, 1000, 104, 10⁵, 10⁶, 10⁷, 10⁸, 10⁹, 10¹⁰, 10¹¹, 10¹², 10¹³, or 10¹⁴, transducing units per target cell per ml of blood are administered to the subject.

1. Viral Particles

Provided herein are viral particles that comprise CD24 or a biologically active portion thereof on an exposed surface of the particle. In some cases the viral particles also comprise CD47 or a biologically active portion thereof on an exposed surface of the particle.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV).

Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740)

Methods of lentiviral transduction are known. Exemplary methods are described in, e.g., Wang et al., J. Immunother. 35(9): 689-701, 2012; Cooper et al., Blood. 101:1637-1644, 2003; Verhoeyen et al., Methods Mol Biol. 506: 97-114, 2009; and Cavalieri et al., Blood. 102(2): 497-505, 2003.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used. In some embodiments, the virus particles are derived from lentivirus. In some embodiments, the lentiviral vector particle is Human Immunodeficiency Virus-1 (HIV-1).

In some cases, the non-cell particle is a viral particle that is morphologically indistinguishable from the wild type infectious virus. In some embodiments, the viral particle presents the entire viral proteome as an antigen.

In some embodiments, the viral particle such as retrovirus or retrovirus-like particle, comprises one or more of gag polyprotein, polymerase (e.g., pol), integrase (e.g., a functional or non-functional variant), protease, and a fusogen. In some embodiments, the non-cell particle further comprises rev. In some embodiments, one or more of the aforesaid proteins are encoded in the retroviral genome, and in some embodiments, one or more of the aforesaid proteins are provided in trans, e.g., by a helper cell, helper virus, or helper plasmid. In some embodiments, the non-cell particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments the non-cell particle nucleic acid further comprises one or more insulator element. In some embodiments, the recognition sites are situated between the poly A tail sequence and the WPRE.

In some embodiments, the viral particle is derived from adenovirus or adenoassociated virus (AAV). In some embodiments, the AAV vector is of serotype 1, 2, 6, 8 or 9. In some embodiments, the AAV vector is of serotype 6.2.

In some embodiments, the AAV vector includes a capsid that is a chimera between AAV2 (aa 1-128) and AAV5 (aa 129-725) with one point mutation (A581T) (AAV2.5T, Excoffon et al. Proc Natl Acad Sci. 106(10):3875-70, 2009).

The AAV is a single-stranded DNA parvovirus which is capable of host genome integration during the latent phase of infectivity. For example, AAV of serotype 2 is largely endemic to the human and primate populations and frequently integrates site-specifically into human chromosome 19 q13.3.

In some aspects, AAV is considered a dependent virus because it requires helper functions from either adenovirus or herpes-virus in order to replicate. In the absence of either of these helper viruses, AAV has been observed to integrate its genome into the host cell chromosome. However, these virions are not capable of propagating infection to new cells.

In some embodiment, suitable host cells for producing AAV derived vehicles include microorganisms, yeast cells, insect cells, and mammalian cells. In some embodiments, the term host cell includes the progeny of the original cell which has been transfected. Thus, as indicated above, a “host cell,” or “producer cell,” as used herein, generally refers to a cell which has been transfected with a vector vehicle as described herein. For example, cells from the stable human cell line, 293 (ATCC Accession No. CRL1573) are familiar to those in the art as a producer cell for AAV vectors. The 293 cell line is a human embryonic kidney cell line that has been transformed with adenovirus type-5 DNA fragments (Graham et al., J. Gen. Virol., 36:59 (1977)), and expresses the adenoviral E1a and E1b genes (Aiello et al., Virol., 94:460 (1979)). The 293 cell line is readily transfected, and thus provides a particularly useful system in which to produce AAV virions.

Producer cells as described above containing the AAV vehicles provided herein must be rendered capable of providing AAV helper functions. In some embodiments, producer cells allow AAV vectors to replicate and encapsulate polynucleotide sequences, such as those encoding a CD24 and/or CD27 associated particle. In some embodiments, producer cells yield AAV virions. AAV helper functions are generally AAV-derived coding sequences that may be expressed to provide AAV gene products that, in turn, function for productive AAV replication.

In some embodiments, AAV helper functions are used to complement necessary AAV functions that are missing from the AAV vectors. In some embodiments, AAV helper functions include at least one of the major AAV ORFs. In some embodiments, the helper functions include at least the rep coding region, or a functional homolog thereof. In some embodiments, the helper function includes at least the cap coding region, or a functional homolog thereof.

In some embodiments, the AAV helper functions are introduced into the host cell by transfecting the host cell with a mixture of AAV helper constructs either prior to, or concurrently with, the transfection of the AAV vector. In some embodiments, the AAV helper constructs are used to provide transient expression of AAV rep and/or cap genes. In some embodiments, the AAV helper constructs lack AAV packaging sequences and can neither replicate nor package themselves.

In some embodiments, an AAV genome can be cross-packaged with a heterologous virus. Cross-genera packing of the rAAV2 genome into the human bocavirus type 1 (HBoV1) capsid (rAAV2/HBoV1 hybrid vector), for example, results in a hybrid vector that is highly tropic for airway epithelium (Yan et al., 2013, Mol. Ther., 21:2181-94).

A number of preclinical studies have demonstrated therapeutic and prophylactic efficacy of viral vector based gene delivery in animal models and in clinical trials. In some aspects, viral and virally derived vectors capable of replication provide consistent gene expression over time. In some aspects, replication competent viruses can result in undesired immunogenicity, toxicity, and cell death. In some embodiments, vectors capable of insertion are efficient for transduction of a variety of cells. However, in some aspects, they can pose a risk of insertional mutagenesis. Integration-deficient vectors can persist episomaly but can also retain the transduction efficiency of standard integrating vectors. Thus, in some embodiments, the vector particle is replication deficient. In some embodiments, the vector particle is integration deficient. Various methods of rendering a vector insertional or replication deficient are known in the art. Various replication-defective vaccine vectors have been produced with many other viruses, including adeno-associated virus (AAV), poliovirus, and Sendai virus.

2. Virus-Like Particles

Also provided herein are virus-like particles (VLPs) that comprise CD24 or a biologically active portion thereof on an exposed surface of the particle. In some embodiments, the virus-like particles comprise CD24 or a biologically active portion thereof and CD47 or a biologically active portion thereof, each on an exposed surface of the particle.

The VLPs include those derived from retroviruses or lentiviruses. While VLPs mimic native virion structure, they lack the viral genomic information necessary for independent replication within a host cell. Therefore, in some aspects, VLPs are non-infectious. In some embodiments, the VLP's bilayer of amphipathic lipids is or comprises the viral envelope. In some embodiments, the particle's bilayer of amphipathic lipids is or comprises lipids derived from a cell. A VLP typically comprises at least one type of structural protein from a virus. In most cases this protein will form a proteinaceous capsid (e.g. VLPs comprising a lentivrus, adenovirus or paramyxovirus structural protein). In some cases the capsid will also be enveloped in a lipid bilayer originating from the cell from which the assembled VLP has been released (e.g.

VLPs comprising a human immunodeficiency virus structural protein such as GAG). In some embodiments, the VLP further comprises a targeting moiety as an envelope protein within the lipid bilayer.

In some embodiments, the non-cell particle comprises supramolecular complexes formed by viral proteins that self-assemble into capsids. In some embodiments, the non-cell particle is a virus-like particle derived from viral capsid proteins. In some embodiments, the non-cell particle is a virus-like particle derived from viral nucleocapsid proteins. In some embodiments, the non-cell particle comprises nucleocapsid-derived proteins that retain the property of packaging nucleic acids. In some embodiments, the viral-based particles, such as virus-like particles comprises only viral structural glycoproteins. In some embodiments, the non-cell particle does not contain a viral genome.

In some embodiments, the non-cell particle packages nucleic acids from host cells during the expression process. In some embodiments, the nucleic acids do not encode any genes involved in virus replication. In particular embodiments, the non-cell particle is a virus-like particle, e.g. retrovirus-like particle such as a lentivirus-like particle, that is replication defective.

In some embodiments, the non-cell particle is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles could be used to deliver therapeutic RNA, in which case functional integrase and/or reverse transcriptase is not required. In some embodiments, the vector particles could also be used to deliver a therapeutic gene of interest, in which case pol is typically included.

3. Methods of Generating Viral-Based Particles

The provided viral-based particles include particles derived from a virus, such as viral particles or virus-like particles, including those derived from retroviruses or lentiviruses.

In some embodiments, the viral particle or virus-like particle is produced from virus family members comprising Parvoviridae (e.g. adeno-associated virus), Retroviridae (e.g. HIV), Flaviviridae (e.g. Hepatitis C virus), Paramyxoviridae (e.g. Nipah) and bacteriophages.

In some embodiments, the viral particle or virus-like particle is produced utilizing proteins (e.g., envelope proteins) from a virus within the Paramyxoviridae family. In some embodiments, the Paramyxoviridae family comprises members within the Henipavirus genus. In some embodiments, the Henipavirus is or comprises a Hendra (HeV) or a Nipah (NiV) virus. In particular embodiments, the viral particles or virus-like particles incorporate a fusogen, such as a targeted envelope protein and fusogen as described in Section IV.

In some embodiments, viral particles or virus-like particles may be produced in multiple cell culture systems including bacteria, mammalian cell lines, insect cell lines, yeast and plant cells.

In some embodiments, the assembly of a viral particle or virus-like particle is initiated by binding of the core protein to a unique encapsidation sequence within the viral genome (e.g. UTR with stem-loop structure). In some embodiments, the interaction of the core with the encapsidation sequence facilitates oligomerization.

Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred.

Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

In some embodiments, a viral vector comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In some embodiments, a viral vector comprises e.g., a virus or viral particle capable of transferring a nucleic acid into a cell, or to the transferred nucleic acid (e.g., as naked DNA). In some embodiments, a viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, the virus particle of viral-like particle, such as a retrovirus or retroviral-like particle (e.g. a lentivirus or lentiviral-like particle) is pseudotyped. In some examples, a pseudotyped virus of viral-like particle has a modification to one or more of its envelope proteins, e.g., an envelope protein is substituted with an envelope protein from another virus. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells.

In some embodiments, retroviral envelope proteins, e.g. lentiviral envelope proteins, are pseudotyped with VSV-G. In one embodiment, source cells produce recombinant retrovirus or retrovirus-like particles, e.g., lentivirus or lentiviral-like particles, pseudotyped with the VSV-G envelope glycoprotein.

In one embodiment, source cells produce recombinant retrovirus or retrovirus-like particles, e.g., lentivirus or lentiviral-like particles, pseudotyped with the VSV-G envelope glycoprotein. In some embodiments, retroviral envelope proteins, e.g. lentiviral envelope proteins, are pseudotyped with an envelope glycoprotein G or H of a virus of the Paramyxoviridae family. In some embodiments, the virus of the Paramyxovirus family is a Henipavirus or is a Morbillivirus. In some aspects, the envelope glycoprotein a Nipah virus G (Niv-G) protein. In other aspects, the envelope glycoprotein is a Hendra virus G protein. In some embodiment, source cells produce recombinant retrovirus or retrovirus-like particles, e.g., lentivirus or lentiviral-like particles, pseudotyped with the envelope glycoprotein G or H of a virus of the Paramyxoviridae family.

In some embodiments, in the vectors described herein at least part of one or more protein coding regions that contribute to or are essential for replication may be absent compared to the corresponding wild-type virus. In some embodiments, the viral vector replication-defective. In some embodiments, the vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome.

In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit vectors to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory.

Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease4 (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a letivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332).

In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rβgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

Some lentiviral vectors integrate inside active genes and possess strong splicing and polyadenylation signals that could lead to the formation of aberrant and possibly truncated transcripts.

Mechanisms of proto-oncogene activation may involve the generation of chimeric transcripts originating from the interaction of promoter elements or splice sites contained in the genome of the insertional mutagen with the cellular transcriptional unit targeted by integration (Gabriel et al. 2009. Nat Med 15: 1431-1436; Bokhoven, et al. J Virol 83:283-29). Chimeric fusion transcripts comprising vector sequences and cellular mRNAs can be generated either by read-through transcription starting from vector sequences and proceeding into the flanking cellular genes, or vice versa.

In some embodiments, a lentiviral nucleic acid described herein comprises a lentiviral backbone in which at least two of the splice sites have been eliminated, e.g., to improve the safety profile of the lentiviral vector. Species of such splice sites and methods of identification are described in WO2012156839A2, all of which is included by reference.

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. The collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid

In some embodiments, the retroviral vector systems described herein comprise viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. In some embodiments, by separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

In some embodiments, a viral vector particle which comprises a sequence that is devoid of or lacking viral RNA may be the result of removing or eliminating the viral RNA from the sequence. In one embodiment this may be achieved by using an endogenous packaging signal binding site on gag. In some embodiments, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles are used to deliver therapeutic RNA, in which case functional integrase and/or reverse transcriptase is not required. In some embodiments, the vector particles could also be used to deliver a therapeutic gene of interest, in which case pol is typically included.

In some embodiments, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

In some embodiments, an alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognising a corresponding sequence in an RNA sequence to facilitate packaging of the RNA sequence into a viral vector particle.

In some embodiments, the heterologous RNA binding domain comprises an RNA binding domain derived from a bacteriophage coat protein, a Rev protein, a protein of the U1 small nuclear ribonucleoprotein particle, a Nova protein, a TF111A protein, a TIS11 protein, a trp RNA-binding attenuation protein (TRAP) or a pseudouridine synthase.

When a recombinant plasmid and the retroviral LTR and packaging sequences are introduced into a producer cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a producer cell line, such as an exemplary HEK 293T cell line, by introduction of plasmids to allow generation of lentiviral particles. In some embodiments, a producer cell is transfected and/or contains a polynucleotide encoding gag and pol, and, in some cases, a polynucleotide encoding an exogenous agent. In some embodiments, the producer cell line is optionally and/or additionally transfected with and/or contains a polynucleotide encoding a rev protein. In some embodiments, the producer cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

In some embodiments, a method herein comprises detecting or confirming the absence of replication competent retrovirus. The methods may include assessing RNA levels of one or more target genes, such as viral genes, e.g. structural or packaging genes, from which gene products are expressed in certain cells infected with a replication-competent retrovirus, such as a gammaretrovirus or lentivirus, but not present in a viral vector used to transduce cells with a heterologous nucleic acid and not, or not expected to be, present and/or expressed in cells not containing replication-competent retrovirus. Replication competent retrovirus may be determined to be present if RNA levels of the one or more target genes is higher than a reference value, which can be measured directly or indirectly, e.g. from a positive control sample containing the target gene. For further disclosure, see WO2018023094A1.

B. Cell-Derived Particles

In some embodiments, the non-cell particle comprises a naturally derived membrane.

In some embodiments, the naturally derived membrane comprises membrane vesicles prepared from cells or tissues. In some embodiments, the non-cell particle comprises a vesicle that is obtainable from a cell. In some embodiments, the non-cell particle comprises a microvesicle, an exosome, a membrane enclosed body, an apoptotic body (from apoptotic cells), a particle (which may be derived from e.g. platelets), an ectosome (derivable from, e.g., neutrophiles and monocytes in serum), a prostatosome (obtainable from prostate cancer cells), or a cardiosome (derivable from cardiac cells).

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeEa, HEK293, HFF-1, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell.

In some embodiments, the non-cell particle has a density of <1, 1-1.1, 1.05-1.15, 1.1-1.2, 1.15-1.25, 1.2-1.3, 1.25-1.35, or >1.35 g/ml. In some embodiments, the non-cell particle composition comprises less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells having a functional nucleus.

In embodiments, the non-cell particle has a size, or the population of non-cell particles have an average size, that is less than about 0.01%, 0.05%, 0.1%, 0.5%, 1%, 2%, 3%, 4%, 5%, 10%, 20%, 30%, 40%, 50%, 60%, 70%, 80%, 90%, of that of the source cell.

In some embodiments the non-cell particle comprises an extracellular vesicle, e.g., a cell-derived vesicle comprising a membrane that encloses an internal space and has a smaller diameter than the cell from which it is derived. In embodiments the extracellular vesicle has a diameter from 20 nm to 1000 nm. In embodiments the non-cell particle comprises an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane). In embodiments the extracellular vesicle is derived from a living or dead organism, explanted tissues or organs, or cultured cells.

In embodiments, the non-cell particle comprises a nanovesicle, e.g., a cell-derived small (e.g., between 20-250 nm in diameter, or 30-150 nm in diameter) vesicle comprising a membrane that encloses an internal space, and which is generated from said cell by direct or indirect manipulation. The production of nanovesicles can, in some instances, result in the destruction of the source cell. The nanovesicle may comprise a lipid or fatty acid and polypeptide.

In embodiments, the non-cell particle comprises an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide. Exemplary exosomes and other membrane-enclosed bodies are also described in WO/2017/161010, WO/2016/077639, US20160168572, US20150290343, and US20070298118, each of which is incorporated by reference herein in its entirety.

In some embodiments, the non-cell particle is a microvesicle. In some embodiments the microvesicle has a diameter of about 100 nm to about 2000 nm.

In some embodiments, a fusosome is a cell ghost. In some embodiments, a vesicle is a plasma membrane vesicle, e.g. a giant plasma membrane vesicle.

In some embodiments, the non-cell particle is derived from a source cell with a genetic modification which results in increased expression of an immunomodulatory agent. In some embodiments, the immunosuppressive agent is on an exterior surface of the cell. In some embodiments, the immunosuppressive agent is incorporated into the exterior surface of the non-cell particle. In some embodiments, the non-cell particle comprises an immunomodulatory agent attached to the surface of the solid particle by a covalent or non-covalent bond.

1. Generation of Cell-Derived Particles

In some embodiments, non-cell particles are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, non-cell particles are generated by inducing cell enucleation. Enucleation may be performed using assays such as genetic, chemical (e.g., using Actinomycin D, see Bayona-Bafaluy et al., “A chemical enucleation method for the transfer of mitochondrial DNA to ρ^{o} cells” Nucleic Acids Res. 2003 Aug. 15; 31(16): e98), mechanical methods (e.g., squeezing or aspiration, see Lee et al., “A comparative study on the efficiency of two enucleation methods in pig somatic cell nuclear transfer: effects of the squeezing and the aspiration methods.” Anim Biotechnol. 2008; 19(2):71-9), or combinations thereof.

In some embodiments, the non-cell particles are generated by inducing cell fragmentation. In some embodiments, cell fragmentation can be performed using the following methods, including, but not limited to: chemical methods, mechanical methods (e.g., centrifugation (e.g., ultracentrifugation, or density centrifugation), freeze-thaw, or sonication), or combinations thereof.

In some embodiments, the source cell used to make the non-cell particle will not be available for testing after the non-cell particle is made.

In some embodiments, a characteristic of a non-cell particle is described by comparison to a reference cell. In embodiments, the reference cell is the source cell. In embodiments, the reference cell is a HeLa, HEK293, HFF-1, MRC-5, WI-38, IMR 90, IMR 91, PER.C6, HT-1080, or BJ cell. In some embodiments, a characteristic of a population of non-cell particles is described by comparison to a population of reference cells, e.g., a population of source cells, or a population of HeLa, HEK293, HFF-1, MRC-5, WI-38, IMR 90, IMR 91, PER.C6, HT-1080, or BJ cells.

C. Synthetic Particles

Also Provided herein are non-cell particles comprising synthetic vesicles. In some embodiments, the synthetic vesicle comprises one or more synthetic lipids. In some embodiments, the non-cell particles comprise synthetic vesicle structures comprising a liposome, a microparticle, a nanoparticle, a nanogel, a dendrimer or a dendrisome.

In some embodiments, synthetic particles comprise nanoparticles. In some embodiments, nanoparticles comprise an inorganic core surrounded by a lipid layer. In some embodiments, the inorganic core is surrounded by a lipid bilayer.

In some embodiments, synthetic particles are engineered solid lipid nanoparticles (SLN) and lipid nano-emulsions (LNE). In some embodiments, SLN and LNE are colloidal nanoparticles with a lipophilic core in a solid state or in a liquid state at room temperature.

In some embodiments, engineered nanoparticles comprise a surface to mass ratio that is much larger than that of other particles. In some embodiments, engineered nanoparticles comprise a relatively large (functional) surface which is able to bind, adsorb and carry other compounds such as drugs, probes and proteins. In some embodiments, nanoparticles comprise dimensions below 0.1 m or 100 nm, especially in the area of drug delivery relatively large (size >100 nm).

In some embodiments, source materials for nanoparticle formation are of biological origin including phospholipids, lipids, lactic acid, dextran, chitosan. In some embodiments, nanoparticles comprise chemical characteristics including various polymers, carbon, silica, and metals.

In some embodiments, synthetic particles are vesicle structures comprising liposomes. In some embodiments, liposomes comprise a uni- or multilamellar lipid bilayer surrounding internal aqueous compartments and a relatively impermeable outer lipophilic phospholipid bilayer. In some embodiments, liposomes are be anionic, neutral or cationic. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

In some embodiments, vesicles or liposomes comprise phospholipids. In some embodiments, vesicles comprise DOPE (dioleoylphosphatidylethanolamine), DOTMA, DOTAP, DOTIM, DDAB, alone or together with cholesterol to yield DOPE and cholesterol, DOTMA and cholesterol, DOTAP and cholesterol, DOTIM and cholesterol, and DDAB and cholesterol. Methods of producing multillamelar vesicles are exemplified in U.S. Pat. No. 6,693,086, incorporated herein by reference. In some embodiments, vesicle formation is spontaneous when a lipid film is mixed with an aqueous solution. In some embodiments, vesicle formation is expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). In some embodiments, vesicle or liposome formation comprises extruded lipids prepared by extruding through filters of decreasing size, as described in Templeton et al., Nature Biotech, 15:647-652, 1997, the teachings of which relating to extruded lipid preparation are incorporated herein by reference.

In some embodiments, additives may be added to vesicles to modify their structure and/or properties. For example, either cholesterol or sphingomyelin may be added to the mixture in order to help stabilize the structure and to prevent the leakage of the inner cargo. In some embodiments, vesicles can be prepared from hydrogenated egg phosphatidylcholine or egg phosphatidylcholine, cholesterol, and dicetyl phosphate, (see, e.g., Spuch and Navarro, Journal of Drug Delivery, vol. 2011, Article ID 469679, 12 pages, 2011. doi: 10.1155/2011/469679 for review). In some embodiments, vesicles may be surface modified during or after synthesis to include reactive groups complementary to the reactive groups on the carrier cells. In some embodiments, reactive groups include maleimide groups. In some embodiments, vesicles may be synthesized to include maleimide conjugated phospholipids such as DSPE-MaL-PEG2000.

In some embodiments, a vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. In some embodiments, manipulation of the lipid membrane with cholesterol reduces rapid release of the encapsulated bioactive compound into the plasma or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In some of any embodiments, lipids are used to form lipid particles. In some embodiments, lipids include, but are not limited to, DLin-KC2-DMA4, CI 2-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. In some embodiments, the component molar ratio may be about 50/10/38.5/1.5 (DLin-KC2-DMA or C12-200/disteroylphosphatidyl choline/cholesterol/PEG-DMG).

In some embodiments, vesicles and lipid-coated polymer particles are able to spontaneously adsorb to cell surfaces.

In some embodiments, particles are comprised of one or more solidified polymer(s) that is arranged in a random manner. In some embodiments, the particles are biodegradable. In some embodiments, biodegradable particles may be synthesized by limitation solvent evaporation, hot melt microencapsulation, solvent removal, and spray drying. Exemplary methods for synthesizing particles are described by Bershteyn et al., Soft Matter 4:1787-1787, 2008 and in US 2008/0014144 A1, the specific teachings of which relating to particle synthesis are incorporated herein by reference. Exemplary synthetic polymers which can be used to form the biodegradable particles include without limitation aliphatic polyesters, poly (lactic acid) (PLA), poly (glycolic acid) (PGA), copolymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

In some embodiments, the diameter of a microparticles, or the average (mean) micorparticle diameter in a composition, ranges from 0.1-1000 micrometers (m). In some embodiments, their diameter, or their average diameter in a composition, ranges in size from 1-750 m, or from 50-500 m, or from 100-250 m. In some embodiments, their diameter, or their average diameter in a composition, ranges in size from 50-1000 m, from 50-750 m, from 50-500 m, or from 50-250 m. In some embodiments, their diameter, or their average diameter in a composition, ranges in size from 0.05-1000 m, from 10-1000 m, from 100-1000 m, or from 500-1000 m. In some embodiments, their diameter, or their average diameter in a composition, is at or about 0.5 m, at or about 10 m, at or about 50 m, at or about 100 m, at or about 200 m, at or about 300 m, at or about 350 m, at or about 400 m, at or about 450 m, at or about 500 m, at or about 550 m, at or about 600 m, at or about 650 m, at or about 700 m, at or about 750 m, at or about 800 m, at or about 850 m, at or about 900 m, at or about 950 m, or at or about 1000 m, or any value between any of the foregoing. Thus, it is to be understood that although these particles are referred to herein as microparticles, the provided embodiments also encompass nanoparticles as well.

In some embodiments, the particle is a nanoparticle. In some embodiments, their diameter, or their average diameter in a composition, ranges in size from 10-250 nm in size, such as 10-200 nm, 10-150 nm, 10-100 nm, 10-80 nm, 10-50 nm, 50-250 nm, 50-200 nm, 50-150 nm, 50-100 nm, 50-80 nm, 80-250 nm, 80-200 nm, 80-150 nm, 80-100 nm, 100-250 nm, 100-200 nm, 100-150 nm, 150-250 nm, 150-200 nm or 200-250 nm. In some embodiments, their diameter, or their average diameter in a composition, is at or about at or about 10 nm, at or about 20 nm, at or about 30 nm, at or about 40 nm, at or about 50 nm, at or about 60 nm, at or about 80 nm, at or about 90 nm, at or about 100 nm, at or about 125 nm, at or about 150 nm, at or about 175 nm, at or about 200 nm, at or about 225 nm, or at or about 250 nm or any value between any of the foregoing.

In some embodiments, a ligand is conjugated to the surface of the particle via a functional chemical group (carboxylic acids, aldehydes, amines, sulfhydryls and hydroxyls) present on the surface of the particle and present on the ligand to be attached. In some embodiments, functionality may be introduced into the particles during the emulsion preparation of particles, incorporation of stabilizers with functional chemical groups. In some embodiments, functional groups are introduced to the particle during post-particle preparation, by direct crosslinking particles and ligands with homo- or heterobifunctional crosslinkers. This procedure may use a suitable chemistry and a class of crosslinkers (CDI, ED AC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the particles may be synthesized to comprise one or more targeting groups on their exterior surface to target a specific cell or tissue type (e.g., cardiomyocytes). In some embodiments, the targeting groups include without limitation receptors, ligands, antibodies, and the like. These targeting groups bind their partner on the cells' surface. In some embodiments, the particles will integrate into a lipid bilayer that comprises the cell surface and the particles are delivered to the cell.

In some embodiments, the particles may also comprise a lipid bilayer on their outermost surface. In some embodiments, the bilayer may be comprised of one or more lipids of the same or different type. In some embodiments, phospholipids are phosphocholines and phosphoinositols. Specific examples include without limitation DMPC, DOPC, DSPC, and various other lipids such as those described herein for liposomes.

In some embodiments, the vesicles or particles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

In some embodiments, liposomes can be characterized as having vesicular structures with a phospholipid bilayer membrane and an inner aqueous medium. In some embodiments, multilamellar liposomes have multiple lipid layers separated by aqueous medium. In some embodiments, liposomes form spontaneously when phospholipids are suspended in an excess of aqueous solution. In some embodiments, the lipid components undergo self-rearrangement before the formation of closed structures and entrap water and dissolved solutes between the lipid bilayers (Ghosh et al, 1991 Glycobiology 5: 505-10). However, compositions that have different structures in solution than the normal vesicular structure are also encompassed. In some embodiments, the lipids may assume a micellar structure or merely exist as nonuniform aggregates of lipid molecules.

In some embodiments, a synthetic particle described herein may include one or more polymers. In some embodiments, the polymers are biodegradable. Exemplary methods for synthesizing polymer vesicles are described by Bershteyn et al., Soft Matter 4:1787-1787, 2008 and in US 2008/0014144 A1, the specific teachings of which relating to particle synthesis are incorporated herein by reference.

In some embodiments, synthetic polymers include without limitation aliphatic polyesters, polyethylene glycol (PEG), poly (lactic acid) (PLA), poly (glycolic acid) (PGA), co-polymers of lactic acid and glycolic acid (PLGA), polycarprolactone (PCL), poly anhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

In some embodiments, the synthetic vesicle comprises a dendrimer. In some embodiments, the dendrimers are nano-sized, radially symmetric molecules with well-defined, homogeneous, and monodisperse structure consisting of tree-like arms or branches. In some embodiments, the dendrimer is an amphiphilic dendrimer constructed by inserting a lipid derivative and cholesterol, named as dendrisome into the vesicle.

In some embodiments, modifications of dendrimers' peripheral groups enable to obtain antibody-dendrimer, peptide-dendrimer conjugates or dendritic boxes that encapsulate guest molecules. In some embodiments, dendrimers are capable of self-assembly. In some embodiments, self-assembly is the spontaneous, precise association of chemical species by specific, complementary intermolecular forces.

### IV. FUSOGENS AND NON-CELL PARTICLE TARGETING AND RETARGETING

In some embodiments, the non-cell particle comprises one or more fusogens. In some embodiments, the fusogen facilitates the fusion of the non-cell particle to a membrane. In some embodiments, the membrane is a plasma cell membrane.

In some embodiments, fusogens comprise protein based, lipid based, and chemical based fusogens. In some embodiments, the non-cell particle comprises a first fusogen comprising a protein fusogen and a second fusogen comprising a lipid fusogen or chemical fusogen. In some embodiments, the fusogen binds fusogen binding partner on a target cells' surface.

In some embodiments, the non-cell particle comprising the fusogen integrates into the membrane into a lipid bilayer of a target cell. In some embodiments, one or more of the fusogens described herein may be included in the non-cell particle.

A. Protein Fusogens

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen results in mixing between lipids in the non-cell particle and lipids in the target cell. In some embodiments, the fusogen results in formation of one or more pores between the interior of the non-cell particle and the cytosol of the target cell.

1. Mammalian Proteins

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/naturel2343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells, any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

2. Viral Proteins

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatatis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gpl60, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glyoproteins F1 and F2; membrane glycoproteins from Venezuelan equine encephalomyelitis; paramyxovirus F protein; SIV gpl60 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g.

Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and R sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

a. G Proteins

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein or a biologically active portion thereof. A non-limited list of exemplary G proteins is shown in Table 1.

The attachment G proteins are type II transmembrane glycoproteins containing an N-terminal cytoplasmic tail (e.g. corresponding to amino acids 1-49 of SEQ ID NO:19), a transmembrane domain (e.g. corresponding to amino acids 50-70 of SEQ ID NO:19, and an extracellular domain containing an extracellular stalk (e.g. corresponding to amino acids 71-187 of SEQ ID NO:19), and a globular head (corresponding to amino acids 188-602 of SEQ ID NO:19). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors eprhin B2 and ephrin B3, but is dispensable for membrane fusion (Brandel-Tretheway et al. Journal of Virology. 2019. 93(13)e00577-19).

In particular embodiments herein, tropism of the G protein is modified. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described below, a re-targeted lipid particle can contain heterologous proteins from different species.

TABLE 1
Exemplary Henipavirus G Proteins

| | | | SEQ ID |
|---|---|---|---|
| | | | NO |
| | | SEQ | (without |
| Viral G | | ID | N-terminal |
| Protein | Sequence | NO | methionine) |
| Hendra | MMADSKLVSLNNNLSGKIKD | 20 | 21 |
| Virus G | QGKVIKNYYGTMDIKKINDG | | |
| Protein | LLDSKILGAFNTVIALLGSI | | |
| | IIIVMNIMIIQNYTRTTDNQ | | |
| | ALIKESLQSVQQQIKALTDK | | |
| | IGTEIGPKVSLIDTSSTITI | | |
| | PANIGLLGSKISQSTSSINE | | |
| | NVNDKCKFTLPPLKIHECNI | | |
| | SCPNPLPFREYRPISQGVSD | | |
| | LVGLPNQICLQKTTSTILKP | | |
| | RLISYTLPINTREGVCITDP | | |
| | LLAVDNGFFAYSHLEKIGSC | | |
| | TRGIAKQRIIGVGEVLDRGD | | |
| | KVPSMFMTNVWTPPNPSTIH | | |
| | HCSSTYHEDFYYTLCAVSHV | | |
| | GDPILNSTSWTESLSLIRLA | | |
| | VRPKSDSGDYNQKYIAITKV | | |
| | ERGKYDKVMPYGPSGIKQGD | | |
| | TLYFPAVGFLPRTEFQYNDS | | |
| | NCPIIHCKYSKAENCRLSMG | | |
| | VNSKSHYILRSGLLKYNLSL | | |
| | GGDIILQFIEIADNRLTIGS | | |
| | PSKIYNSLGQPVFYQASYSW | | |
| | DTMIKLGDVDTVDPLRVQWR | | |
| | NNSVISRPGQSQCPRFNVCP | | |
| | EVCWEGTYNDAFLIDRLNWV | | |
| | SAGVYLNSNQTAENPVFAVF | | |
| | KDNEILYQVPLAEDDTNAQK | | |
| | TITDCFLLENVIWCISLVEI | | |
| | YDTGDSVIRPKLFAVKIPAQ | | |
| | CSES | | |
| Nipah | MPAENKKVRFENTTSDKGKI | 22 | 23 |
| Virus G | PSKVIKSYYGTMDIKKINEG | | |
| Protein | LLDSKILSAFNTVIALLGSI | | |
| | VIIVMNIMIIQNYTRSTDNQ | | |
| | AVIKDALQGIQQQIKGLADK | | |
| | IGTEIGPKVSLIDTSSTITI | | |
| | PANIGLLGSKISQSTASINE | | |
| | NVNEKCKFTLPPLKIHECNI | | |
| | SCPNPLPFREYRPQTEGVSN | | |
| | LVGLPNNICLQKTSNQILKP | | |
| | KLISYTLPVVGQSGTCITDP | | |
| | LLAMDEGYFAYSHLERIGSC | | |
| | SRGVSKQRIIGVGEVLDRGD | | |
| | EVPSLFMTNVWTPPNPNTVY | | |
| | HCSAVYNNEFYYVLCAVSTV | | |
| | GDPILNSTYWSGSLMMTRLA | | |
| | VKPKSNGGGYNQHQLALRSI | | |
| | EKGRYDKVMPYGPSGIKQGD | | |
| | TLYFPAVGFLVRTEFKYNDS | | |
| | NCPITKCQYSKPENCRLSMG | | |
| | IRPNSHYILRSGLLKYNLSD | | |
| | GENPKVVFIEISDQRLSIGS | | |
| | PSKIYDSLGQPVFYQASFSW | | |
| | DTMIKFGDVLTVNPLVVNWR | | |
| | NNTVISRPGQSQCPRFNTCP | | |
| | EICWEGVYNDAFLIDRINWI | | |
| | SAGVFLDSNQTAENPVFTVF | | |
| | KDNEILYRAQLASEDTNAQK | | |
| | TITNCFLLKNKIWCISLVEI | | |
| | YDTGDNVIRPKLFAVKIPEQ | | |
| | CT | | |
| Cedar | MLSQLQKNYLDNSNQQGDKM | 24 | 25 |
| Virus G | NNPDKKLSVNFNPLELDKGQ | | |
| Protein | KDLNKSYYVKNKNYNVSNLL | | |
| | NESLHDIKFCIYCIFSLLII | | |
| | ITIINIITISIVITRLKVHE | | |
| | ENNGMESPNLQSIQDSLSSL | | |
| | TNMINTEITPRIGILVTATS | | |
| | VTLSSSINYVGTKTNQLVNE | | |
| | LKDYITKSCGFKVPELKLHE | | |
| | CNISCADPKISKSAMYSTNA | | |
| | YAELAGPPKIFCKSVSKDPD | | |
| | FRLKQIDYVIPVQQDRSICM | | |
| | NNPLLDISDGFFTYIHYEGI | | |
| | NSCKKSDSFKVLLSHGEIVD | | |
| | RGDYRPSLYLLSSHYHPYSM | | |
| | QVINCVPVTCNQSSFVFCHI | | |
| | SNNTKTLDNSDYSSDEYYIT | | |
| | YFNGIDRPKTKKIPINNMTA | | |
| | DNRYIHFTFSGGGGVCLGEE | | |
| | FIIPVTTVINTDVFTHDYCE | | |
| | SFNCSVQTGKSLKEICSESL | | |
| | RSPTNSSRYNLNGIMIISQN | | |
| | NMTDFKIQLNGITYNKLSFG | | |
| | SPGRLSKTLGQVLYYQSSMS | | |
| | WDTYLKAGFVEKWKPFTPNW | | |
| | MNNTVISRPNQGNCPRYHKC | | |
| | PEICYGGTYNDIAPLDLGKD | | |
| | MYVSVILDSDQLAENPEITV | | |
| | FNSTTILYKERVSKDELNTR | | |
| | STTTSCFLFLDEPWCISVLE | | |
| | TNRFNGKSIRPEIYSYKIPK | | |
| | YC | | |
| Bat | MPQKTVEFINMNSPLERGVS | 26 | 27 |
| Paramyxo- | TLSDKKTLNQSKITKQGYFG | | |
| virus G | LGSHSERNWKKQKNQNDHYM | | |
| Protein, | TVSTMILEILVVLGIMFNLI | | |
| Eid_hel/ | VLTMVYYQNDNINQRMAELT | | |
| GHM74a/ | SNITVLNLNLNQLTNKIQRE | | |
| GHA/2009 | IIPRITLIDTATTITIPSAI | | |
| | TYILATLTTRISELLPSINQ | | |
| | KCEFKTPTLVLNDCRINCTP | | |
| | PLNPSDGVKMSSLATNLVAH | | |
| | GPSPCRNFSSVPTIYYYRIP | | |
| | GLYNRTALDERCILNPRLTI | | |
| | SSTKFAYVHSEYDKNCTRGF | | |
| | KYYELMTFGEILEGPEKEPR | | |
| | MFSRSFYSPTNAVNYHSCTP | | |
| | IVTVNEGYFLCLECTSSDPL | | |
| | YKANLSNSTFHLVILRHNKD | | |
| | EKIVSMPSFNLSTDQEYVQI | | |
| | IPAEGGGTAESGNLYFPCIG | | |
| | RLLHKRVTHPLCKKSNCSRT | | |
| | DDESCLKSYYNQGSPQHQVV | | |
| | NCLIRIRNAQRDNPTWDVIT | | |
| | VDLTNTYPGSRSRIFGSFSK | | |
| | PMLYQSSVSWHTLLQVAEIT | | |
| | DLDKYQLDWLDTPYISRPGG | | |
| | SECPFGNYCPTVCWEGTYND | | |
| | VYSLTPNNDLFVTVYLKSEQ | | |
| | VAENPYFAIFSRDQILKEFP | | |
| | LDAWISSARTTTISCFMFNN | | |
| | EIWCIAALEITRLNDDIIRP | | |
| | IYYSFWLPTDCRTPYPHTGK | | |
| | MTRVPLRSTYNY | | |
| Mojiang | MATNRDNTITSAEVSQEDKV | 28 | 29 |
| virus, | KKYYGVETAEKVADSISGNK | | |
| Tongguan | VFILMNTLLILTGAIITITL | | |
| 1 G | NITNLTAAKSQQNMLKIIQD | | |
| Protein | DVNAKLEMFVNLDQLVKGEI | | |
| | KPKVSLINTAVSVSIPGQIS | | |
| | NLQTKFLQKYVYLEESITKQ | | |
| | CTCNPLSGIFPTSGPTYPPT | | |
| | DKPDDDTTDDDKVDTTIKPI | | |
| | EYPKPDGCNRTGDHFTMEPG | | |
| | ANFYTVPNLGPASSNSDECY | | |
| | TNPSFSIGSSIYMFSQEIRK | | |
| | TDCTAGEILSIQIVLGRIVD | | |
| | KGQQGPQASPLLVWAVPNPK | | |
| | IINSCAVAAGDEMGWVLCSV | | |
| | TLTAASGEPIPHMFDGFWLY | | |
| | KLEPDTEVVSYRITGYAYLL | | |
| | DKQYDSVFIGKGGGIQKGND | | |
| | LYFQMYGLSRNRQSFKALCE | | |
| | HGSCLGTGGGGYQVLCDRAV | | |
| | MSFGSEESLITNAYLKVNDL | | |
| | ASGKPVIIGQTFPPSDSYKG | | |
| | SNGRMYTIGDKYGLYLAPSS | | |
| | WNRYLRFGITPDISVRSTTW | | |
| | LKSQDPIMKILSTCTNTDRD | | |
| | MCPEICNTRGYQDIFPLSED | | |
| | SEYYTYIGITPNNGGTKNFV | | |
| | AVRDSDGHIASIDILQNYYS | | |
| | ITSATISCFMYKDEIWCIAI | | |
| | TEGKKQKDNPQRIYAHSYKI | | |
| | RQMCYNMKSATVTVGNAKNI | | |
| | TIRRY | | |

In some embodiments, the G protein has a sequence set forth in any of SEQ ID NOS: 19-29 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 19, 20, 21, 22, 23, 24, 25, 26, 27, 28 or 29.

In particular embodiments, the G protein or functionally active variant or biologically active portion is a protein that retains fusogenic activity in conjunction with a Henipavirus F protein, e.g. NiV-F or HeV-F. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

In particular embodiments, the G protein has the sequence of amino acids set forth in SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 or SEQ ID NO: 29 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 or SEQ ID NO: 29 and retains fusogenic activity in conjunction with a Henipavirus F protein (e.g., NiV-F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 or SEQ ID NO: 29 and retains fusogenic activity in conjunction with a Henipavirus F protein (e.g., NiV-F or HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 or SEQ ID NO: 29 such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 or SEQ ID NO: 29.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 or SEQ ID NO: 29. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:19, SEQ ID NO:22 or SEQ ID NO:23, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:19, SEQ ID NO:22 or SEQ ID NO:23.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:19, SEQ ID NO:22 or SEQ ID NO:23), up to 6 contiguous amino acid residues at or near the N-terminus of the wild-type NiV-G protein (SEQ ID NO:19, SEQ ID NO:22 or SEQ ID NO:23), up to 7 contiguous amino acid residues at or near the N-terminus of the wild-type NiV-G protein (SEQ ID NO:19, SEQ ID NO:22 or SEQ ID NO:23), up to 8 contiguous amino acid residues at or near the N-terminus of the wild-type NiV-G protein (SEQ ID NO:19, SEQ ID NO:22 or SEQ ID NO:23), up to 9 contiguous amino acid residues at or near the N-terminus of the wild-type NiV-G protein (SEQ ID NO:19, SEQ ID NO:22 or SEQ ID NO:23) up to 10 contiguous amino acid residues at or near the N-terminus of the wild-type NiV-G protein (SEQ ID NO:19, SEQ ID NO:22 or SEQ ID NO:23), up to 11 contiguous amino acid residues at or near the N-terminus of the wild-type NiV-G protein (SEQ ID NO:19, SEQ ID NO:22 or SEQ ID NO:23), up to 12 contiguous amino acid residues at or near the N-terminus of the wild-type NiV-G protein SEQ ID NO:23, SEQ ID NO:19, or SEQ ID NO:22), up to 13 contiguous amino acid residues at or near the N-terminus of the wild-type NiV-G protein (SEQ ID NO:19, SEQ ID NO:22 or SEQ ID NO:23), up to 14 contiguous amino acid residues at or near the N-terminus of the wild-type NiV-G protein (SEQ ID NO:19, SEQ ID NO:22 or SEQ ID NO:23), up to 15 contiguous amino acid residues at or near the N-terminus of the wild-type NiV-G protein (SEQ ID NO:19, SEQ ID NO:22 or SEQ ID NO:23) up to 16 contiguous amino acid residues at or near the N-terminus of the wild-type NiV-G protein (SEQ ID NO:19, SEQ ID NO:22 or SEQ ID NO:23), up to 17 contiguous amino acid residues at or near the N-terminus of the wild-type NiV-G protein (SEQ ID NO:19, SEQ ID NO:22 or SEQ ID NO:23), up to 18 contiguous amino acid residues at or near the N-terminus of the wild-type NiV-G protein (SEQ ID NO:19, SEQ ID NO:22 or SEQ ID NO:23), up to 19 contiguous amino acid residues at or near the N-terminus of the wild-type NiV-G protein (SEQ ID NO:19, SEQ ID NO:22 or SEQ ID NO:23), up to 20 contiguous amino acid residues at or near the N-terminus of the wild-type NiV-G protein (SEQ ID NO:19, SEQ ID NO:22 or SEQ ID NO:23), up to 21 contiguous amino acid residues at or near the N-terminus of the wild-type NiV-G protein SEQ ID NO:19, SEQ ID NO:22 or SEQ ID NO:23), up to 22 contiguous amino acid residues at or near the N-terminus of the wild-type NiV-G protein (SEQ ID NO:19, SEQ ID NO:22 or SEQ ID NO:23), up to 23 contiguous amino acid residues at or near the N-terminus of the wild-type NiV-G protein (SEQ ID NO:19, SEQ ID NO:22 or SEQ ID NO:23), up to 24 contiguous amino acid residues at or near the N-terminus of the wild-type NiV-G protein (SEQ ID NO:19, SEQ ID NO:22 or SEQ ID NO:23), up to 25 contiguous amino acid residues at or near the N-terminus of the wild-type NiV-G protein (SEQ ID NO:19, SEQ ID NO:22 or SEQ ID NO:23), up to 26 contiguous amino acid residues at or near the N-terminus of the wild-type NiV-G protein (SEQ ID NO:19, SEQ ID NO:22 or SEQ ID NO:23), up to 27 contiguous amino acid residues at or near the N-terminus of the wild-type NiV-G protein (SEQ ID NO:19, SEQ ID NO:22 or SEQ ID NO:23), up to 28 contiguous amino acid residues at or near the N-terminus of the wild-type NiV-G protein (SEQ ID NO:19, SEQ ID NO:22 or SEQ ID NO:23), up to 29 contiguous amino acid residues at or near the N-terminus of the wild-type NiV-G protein (SEQ ID NO:19, SEQ ID NO:22 or SEQ ID NO:23), up to 30 contiguous amino acid residues at or near the N-terminus of the wild-type NiV-G protein (SEQ ID NO:19, SEQ ID NO:22 or SEQ ID NO:23), up to 31 contiguous amino acid residues at or near the N-terminus of the wild-type NiV-G protein (SEQ ID NO:19, SEQ ID NO:22 or SEQ ID NO:23), up to 32 contiguous amino acid residues at or near the N-terminus of the wild-type NiV-G protein (SEQ ID NO:19, SEQ ID NO:22 or SEQ ID NO:23), up to 33 contiguous amino acid residues at or near the N-terminus of the wild-type NiV-G protein (SEQ ID NO:19, SEQ ID NO:22 or SEQ ID NO:23), up to 34 contiguous amino acid residues at or near the N-terminus of the wild-type NiV-G protein (SEQ ID NO:19, SEQ ID NO:22 or SEQ ID NO:23), up to 35 contiguous amino acid residues at or near the N-terminus of the wild-type NiV-G protein (SEQ ID NO:19, SEQ ID NO:22 or SEQ ID NO:23), up to 36 contiguous amino acid residues at or near the N-terminus of the wild-type NiV-G protein (SEQ ID NO:19, SEQ ID NO:22 or SEQ ID NO:23), up to 37 contiguous amino acid residues at or near the N-terminus of the wild-type NiV-G protein (SEQ ID NO:19, SEQ ID NO:22 or SEQ ID NO:23) up to 38 contiguous amino acid residues at or near the N-terminus of the wild-type NiV-G protein (SEQ ID NO:19, SEQ ID NO:22 or SEQ ID NO:23), up to 39 contiguous amino acid residues at or near the N-terminus of the wild-type NiV-G protein (SEQ ID NO:19, SEQ ID NO:22 or SEQ ID NO:23), up to 40 contiguous amino acid residues at or near the N-terminus of the wild-type NiV-G protein (SEQ ID NO:19, SEQ ID NO:22 or SEQ ID NO:23), up to 41 contiguous amino acid residues at or near the N-terminus of the wild-type NiV-G protein (SEQ ID NO:19, SEQ ID NO:22 or SEQ ID NO:23), up to 42 contiguous amino acid residues at or near the N-terminus of the wild-type NiV-G protein (SEQ ID NO:19, SEQ ID NO:22 or SEQ ID NO:23), up to 43 contiguous amino acid residues at or near the N-terminus of the wild-type NiV-G protein (SEQ ID NO:19, SEQ ID NO:22 or SEQ ID NO:23), up to 44 contiguous amino acid residues at or near the N-terminus of the wild-type NiV-G protein (SEQ ID NO:19, SEQ ID NO:22 or SEQ ID NO:23), or up to 45 contiguous amino acid residues at or near the N-terminus of the wild-type NiV-G protein (SEQ ID NO:19, SEQ ID NO:22 or SEQ ID NO:23).

In some embodiments, the NiV-G protein is a biologically active portion that does not contain a cytoplasmic domain. In some embodiments, the NiV-G protein without the cytoplasmic domain is encoded by SEQ ID NO: 40.

In some embodiments, the mutant NiV-G protein comprises a sequence set forth in any of SEQ ID NOS: 30-40, or is a functional variant thereof that has an amino acid sequence having at least at or 80%, at least at or about 81%, at least at or about 82%, at least at or about 83%, at or about 84%, at least at or about 85%, at least at or about 86%, or at least at or about 87%, at least at or about 88%, or at least at or about 89%, about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOS: 30-40.

In some embodiments, the mutant NiV-G protein has a 5 amino acid truncation at or near the N-terminus of the wild-type NiV-G protein (SEQ ID NO:30, SEQ ID NO: 31 or SEQ ID NO:32), such as set forth in SEQ ID NO: 33 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:33 or such as set forth in SEQ ID NO: 34 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:34 or such as set forth in SEQ ID NO: 35 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35. In some embodiments, the mutant NiV-G protein has a 10 amino acid truncation at or near the N-terminus of the wild-type NiV-G protein (SEQ ID NO:36, SEQ ID NO:37 or SEQ ID NO:38), such as set forth in SEQ ID NO: 36 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:36, or such as set forth in SEQ ID NO: 37 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:37 or such as set forth in SEQ ID NO: 38 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:38.

In some embodiments, the mutant NiV-G protein has a 15 amino acid truncation at or near the N-terminus of the wild-type NiV-G protein (SEQ ID NO:39, or SEQ ID NO:40), such as set forth in SEQ ID NO: 39 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39 or such as set forth in SEQ ID NO: 40 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:40.

In some embodiments, the G protein is a mutant HeV-G protein that is a biologically active portion of a wild-type HeV-G. In some embodiments, the biologically active portion is an N-terminally truncated fragment.

In some embodiments, the mutant G protein is a mutant HeV-G protein that has the sequence set forth in SEQ ID NO:41 or 42, or is a functional variant or biologically active portion thereof that has an amino acid sequence having at least at or about 80%, at least at or about 81%, at least at or about 82%, at least at or about 83%, at least at or about 84%, at or about 85%, at least at or about 86%, at least at or about 87%, at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:41 or 42.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:41 or 42), up to 6 contiguous amino acid residues at or near the N-terminus of the wild-type HeV-G protein (SEQ ID NO:41 or 42), up to 7 contiguous amino acid residues at or near the N-terminus of the wild-type HeV-G protein (SEQ ID NO:41 or 42 or up to 8 contiguous amino acid residues at or near the N-terminus of the wild-type HeV-G protein (SEQ ID NO:41 or 42), up to 9 contiguous amino acid residues at or near the N-terminus of the wild-type HeV-G protein (SEQ ID NO:41 or 42), up to 10 contiguous amino acid residues at or near the N-terminus of the wild-type HeV-G protein (SEQ ID NO:41 or 42), up to 11 contiguous amino acid residues at or near the N-terminus of the wild-type HeV-G protein (SEQ ID NO:41 or 42), up to 12 contiguous amino acid residues at or near the N-terminus of the wild-type HeV-G protein (SEQ ID NO:41 or 42), up to 13 contiguous amino acid residues at or near the N-terminus of the wild-type HeV-G protein (SEQ ID NO: 41 or 42), up to 14 contiguous amino acid residues at or near the N-terminus of the wild-type HeV-G protein (SEQ ID NO: 41 or 42), up to 15 contiguous amino acid residues at or near the N-terminus of the wild-type HeV-G protein (SEQ ID NO: 41 or 42), up to 16 contiguous amino acid residues at or near the N-terminus of the wild-type HeV-G protein (SEQ ID NO: 41 or 42), up to 17 contiguous amino acid residues at or near the N-terminus of the wild-type HeV-G protein (SEQ ID NO: 41 or 42), up to 18 contiguous amino acid residues at or near the N-terminus of the wild-type HeV-G protein (SEQ ID NO: 41 or 42), up to 19 contiguous amino acid residues at or near the N-terminus of the wild-type HeV-G protein (SEQ ID NO: 41 or 42), up to 20 contiguous amino acid residues at or near the N-terminus of the wild-type HeV-G protein (SEQ ID NO: 41 or 42), up to 21 contiguous amino acid residues at or near the N-terminus of the wild-type HeV-G protein (SEQ ID NO: 41 or 42), up to 22 contiguous amino acid residues at or near the N-terminus of the wild-type HeV-G protein (SEQ ID NO: 41 or 42), up to 23 contiguous amino acid residues at or near the N-terminus of the wild-type HeV-G protein (SEQ ID NO: 41 or 42), up to 24 contiguous amino acid residues at or near the N-terminus of the wild-type HeV-G protein (SEQ ID NO: 41 or 42), up to 25 contiguous amino acid residues at or near the N-terminus of the wild-type HeV-G protein (SEQ ID NO: 41 or 42), up to 26 contiguous amino acid residues at or near the N-terminus of the wild-type HeV-G protein (SEQ ID NO: 41 or 42), up to 27 contiguous amino acid residues at or near the N-terminus of the wild-type HeV-G protein (SEQ ID NO: 41 or 42), up to 28 contiguous amino acid residues at or near the N-terminus of the wild-type HeV-G protein (SEQ ID NO: 41 or 42), up to 29 contiguous amino acid residues at or near the N-terminus of the wild-type HeV-G protein (SEQ ID NO: 41 or 42), up to 30 contiguous amino acid residues at or near the N-terminus of the wild-type HeV-G protein (SEQ ID NO: 41 or 42), up to 31 contiguous amino acid residues at or near the N-terminus of the wild-type HeV-G protein (SEQ ID NO: 41 or 42), up to 32 contiguous amino acid residues at or near the N-terminus of the wild-type HeV-G protein (SEQ ID NO: 41 or 42), up to 33 contiguous amino acid residues at or near the N-terminus of the wild-type HeV-G protein (SEQ ID NO: 41 or 42), up to 34 contiguous amino acid residues at or near the N-terminus of the wild-type HeV-G protein (SEQ ID NO: 41 or 42), up to 35 contiguous amino acid residues at or near the N-terminus of the wild-type HeV-G protein (SEQ ID NO: 41 or 42), up to 36 contiguous amino acid residues at or near the N-terminus of the wild-type HeV-G protein (SEQ ID NO: 41 or 42), up to 37 contiguous amino acid residues at or near the N-terminus of the wild-type HeV-G protein (SEQ ID NO: 41 or 42), up to 38 contiguous amino acid residues at or near the N-terminus of the wild-type HeV-G protein (SEQ ID NO: 41 or 42), up to 39 contiguous amino acid residues at or near the N-terminus of the wild-type HeV-G protein (SEQ ID NO: 41 or 42), up to 40 contiguous amino acid residues at or near the N-terminus of the wild-type HeV-G protein (SEQ ID NO: 41 or 42), up to 41 contiguous amino acid residues at or near the N-terminus of the wild-type HeV-G protein (SEQ ID NO: 41 or 42), up to 42 contiguous amino acid residues at or near the N-terminus of the wild-type HeV-G protein (SEQ ID NO: 41 or 42), up to 43 contiguous amino acid residues at or near the N-terminus of the wild-type HeV-G protein (SEQ ID NO: 41 or 42), up to 44 contiguous amino acid residues at or near the N-terminus of the wild-type HeV-G protein (SEQ ID NO: 41 or 42), or up to 45 contiguous amino acid residues at or near the N-terminus of the wild-type HeV-G protein (SEQ ID NO: 41 or 42). In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO: 41 or 42), such as set forth in SEQ ID NO:44 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:44.

In some embodiments, the G protein or the functionally active variant or biologically active portion thereof binds to Ephrin B2 or Ephrin B3. In some aspects, the G protein has the sequence of amino acids set forth in any one of SEQ ID NO:42, SEQ ID NO:41 or SEQ ID NO:22, SEQ ID NO:24, SEQ ID NO: 23, SEQ ID NO:26 or SEQ ID NO:28, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to any of SEQ ID NO:42, SEQ ID NO:41 or SEQ ID NO:22, SEQ ID NO:24, SEQ ID NO: 23, SEQ ID NO:26 or SEQ ID NO:28, or a functionally active variant or biologically active portion thereof, and retains binding to Ephrhin B2 or B3.

In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, and retains binding to Ephrhin B2 or B3. Reference to retaining binding to Ephrin B2 or B3 includes binding that is at least or at least about 5% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 10% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 15% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 20% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 25% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion, 30% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 35% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 40% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 45% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 50% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 55% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 60% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 65% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, 70% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28 or a functionally active variant or biologically active portion thereof, such as at least or at least about 75% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, such as at least or at least about 80% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, such as at least or at least about 85% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, such as at least or at least about 90% of the level or degree of binding of the corresponding wild-type G protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof, or such as at least or at least about 95% of the level or degree of binding of the corresponding wild-type protein, such as set forth in SEQ ID NO:45, SEQ ID NO:41 or SEQ ID NO:22, SEQ ID NO:24, SEQ ID NO: 23, SEQ ID NO:26 or SEQ ID NO:28, or a functionally active variant or biologically active portion thereof. In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:22, SEQ ID NO:23 or SEQ ID NO:45, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:22, SEQ ID NO:23 or SEQ ID NO:45 and retains binding to Eprhin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type NiV-G, such as set forth in SEQ ID NO:22, SEQ ID NO:23 or SEQ ID NO:45, 10% of the level or degree of binding of the corresponding wild-type NiV-G, such as set forth in SEQ ID NO:22, SEQ ID NO:23 or SEQ ID NO:45, 15% of the level or degree of binding of the corresponding wild-type NiV-G, such as set forth in SEQ ID NO:22, SEQ ID NO:23 or SEQ ID NO:45, 20% of the level or degree of binding of the corresponding wild-type NiV-G, such as set forth in SEQ ID NO:22, SEQ ID NO:23 or SEQ ID NO:45, 25% of the level or degree of binding of the corresponding wild-type NiV-G, such as set forth in SEQ ID NO:22, SEQ ID NO:23 or SEQ ID NO:45, 30% of the level or degree of binding of the corresponding wild-type NiV-G, such as set forth in SEQ ID NO:22, SEQ ID NO:23 or SEQ ID NO:45, 35% of the level or degree of binding of the corresponding wild-type NiV-G, such as set forth in SEQ ID NO:22, SEQ ID NO:23 or SEQ ID NO:45, 40% of the level or degree of binding of the corresponding wild-type NiV-G, such as set forth in SEQ ID NO:22, SEQ ID NO:23 or SEQ ID NO:45, 45% of the level or degree of binding of the corresponding wild-type NiV-G, such as set forth in SEQ ID NO:22, SEQ ID NO:23 or SEQ ID NO:45 50% of the level or degree of binding of the corresponding wild-type NiV-G, such as set forth in SEQ ID NO:22, SEQ ID NO:23 or SEQ ID NO:68, 55% of the level or degree of binding of the corresponding wild-type NiV-G, such as set forth in SEQ ID NO:22, SEQ ID NO:23 or SEQ ID NO:45, 60% of the level or degree of binding of the corresponding wild-type NiV-G, such as set forth in SEQ ID NO:22, SEQ ID NO:23 or SEQ ID NO:45, 65% of the level or degree of binding of the corresponding wild-type NiV-G, such as set forth in SEQ ID NO:22, SEQ ID NO:23 or SEQ ID NO:45, 70% of the level or degree of binding of the corresponding wild-type NiV-G, such as set forth in SEQ ID NO:8, SEQ ID NO:9 or SEQ ID NO:44, such as at least or at least about 75% of the level or degree of binding of the corresponding wild-type NiV-G, such as set forth in SEQ ID NO:22, SEQ ID NO:23 or SEQ ID NO:45, such as at least or at least about 80% of the level or degree of binding of the corresponding wild-type NIV-G, such as set forth in SEQ ID NO:22, SEQ ID NO:23 or SEQ ID NO:45, such as at least or at least about 85% of the level or degree of binding of the corresponding wild-type NiV-G, such as set forth in SEQ ID NO:22, SEQ ID NO:23 or SEQ ID NO:45, such as at least or at least about 90% of the level or degree of binding of the corresponding wild-type NiV-G, such as set forth in SEQ ID NO:22, SEQ ID NO:23 or SEQ ID NO:45, or such as at least or at least about 95% of the level or degree of binding of the corresponding wild-type NiV-G, such as set forth in SEQ ID NO:22, SEQ ID NO:23 or SEQ ID NO:45.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as amutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:41 or 42, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 40 or 41 and retains binding to Eprhin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type HeV-G, such as set forth in SEQ ID NO: 39 or 40, 10% of the level or degree of binding of the corresponding wild-type HeV-G, such as set forth in SEQ ID NO: 39 or 40, 15% of the level or degree of binding of the corresponding wild-type HeV-G, such as set forth in SEQ ID NO:66 or 67, 20% of the level or degree of binding of the corresponding wild-type HeV-G, such as set forth in SEQ ID NO: 41 or 42, 25% of the level or degree of binding of the corresponding wild-type HeV-G, such as set forth in SEQ ID NO: 41 or 42, 30% of the level or degree of binding of the corresponding wild-type HeV-G, such as set forth in SEQ ID NO: 41 or 42, 35% of the level or degree of binding of the corresponding wild-type HeV-G, such as set forth in SEQ ID NO: 41 or 42, 40% of the level or degree of binding of the corresponding wild-type HeV-G, such as set forth in SEQ ID NO: 41 or 42, 45% of the level or degree of binding of the corresponding wild-type HeV-G, such as set forth in SEQ ID NO: 41 or 42, 50% of the level or degree of binding of the corresponding wild-type HeV-G, such as set forth in SEQ ID NO: 41 or 42, 55% of the level or degree of binding of the corresponding wild-type HeV-G, such as set forth in SEQ ID NO: 41 or 42, 60% of the level or degree of binding of the corresponding wild-type HeV-G, such as set forth in SEQ ID NO: 41 or 42, 65% of the level or degree of binding of the corresponding wild-type HeV-G, such as set forth in SEQ ID NO: 41 or 42, 70% of the level or degree of binding of the corresponding wild-type HeV-G, such as set forth in SEQ ID NO:18 or 52, such as at least or at least about 75% of the level or degree of binding of the corresponding wild-type HeV-G, such as set forth in SEQ ID NO: 41 or 42, such as at least or at least about 80% of the level or degree of binding of the corresponding wild-type NIV-G, such as set forth in SEQ ID NO: 41 or 42, such as at least or at least about 85% of the level or degree of binding of the corresponding wild-type HeV-G, such as set forth in SEQ ID NO: 41 or 42, such as at least or at least about 90% of the level or degree of binding of the corresponding wild-type HeV-G, such as set forth in SEQ ID NO: 41 or 42, or such as at least or at least about 95% of the level or degree of binding of the corresponding wild-type HeV-G, such as set forth in SEQ ID NO: 41 or 42.

In some embodiments, the G protein or the biologically thereof is a mutant G protein that exhibits reduced binding for the native binding partner of a wild-type G protein.

In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as amutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:39.

In some embodiments, the G protein is a mutant G protein containing one or more amino acid substitutions selected from the group consisting of E501A, W504A, Q530A and E533A with reference to numbering set forth in SEQ ID NO:22. In some embodiments, the G protein is a mutant G protein that contains one or more amino acid substitutions elected from the group consisting of E501A, W504A, Q530A and E533A with reference to SEQ ID NO:22 and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO: 22), 6 contiguous amino acid residues at or near the N-terminus of the wild-type NiV-G protein (SEQ ID NO: 22), 7 contiguous amino acid residues at or near the N-terminus of the wild-type NiV-G protein (SEQ ID NO: 22), 8 contiguous amino acid residues at or near the N-terminus of the wild-type NiV-G protein (SEQ ID NO: 22), 9 contiguous amino acid residues at or near the N-terminus of the wild-type NiV-G protein (SEQ ID NO: 22), up to 10 contiguous amino acid residues at or near the N-terminus of the wild-type NiV-G protein (SEQ ID NO: 22), 11 contiguous amino acid residues at or near the N-terminus of the wild-type NiV-G protein (SEQ ID NO: 22), 12 contiguous amino acid residues at or near the N-terminus of the wild-type NiV-G protein (SEQ ID NO: 22), 13 contiguous amino acid residues at or near the N-terminus of the wild-type NiV-G protein (SEQ ID NO: 22), 14 contiguous amino acid residues at or near the N-terminus of the wild-type NiV-G protein (SEQ ID NO: 22), up to 15 contiguous amino acid residues at or near the N-terminus of the wild-type NiV-G protein (SEQ ID NO: 22), 16 contiguous amino acid residues at or near the N-terminus of the wild-type NiV-G protein (SEQ ID NO: 22), 17 contiguous amino acid residues at or near the N-terminus of the wild-type NiV-G protein (SEQ ID NO: 22), 18 contiguous amino acid residues at or near the N-terminus of the wild-type NiV-G protein (SEQ ID NO: 22), 19 contiguous amino acid residues at or near the N-terminus of the wild-type NiV-G protein (SEQ ID NO: 22), up to 20 contiguous amino acid residues at or near the N-terminus of the wild-type NiV-G protein (SEQ ID NO: 22), 21 contiguous amino acid residues at or near the N-terminus of the wild-type NiV-G protein (SEQ ID NO: 22) 22 contiguous amino acid residues at or near the N-terminus of the wild-type NiV-G protein (SEQ ID NO: 22), 23 contiguous amino acid residues at or near the N-terminus of the wild-type NiV-G protein (SEQ ID NO: 22), 24 contiguous amino acid residues at or near the N-terminus of the wild-type NiV-G protein (SEQ ID NO: 22), up to 25 contiguous amino acid residues at or near the N-terminus of the wild-type NiV-G protein (SEQ ID NO: 22), 26 contiguous amino acid residues at or near the N-terminus of the wild-type NiV-G protein (SEQ ID NO: 22), 27 contiguous amino acid residues at or near the N-terminus of the wild-type NiV-G protein (SEQ ID NO: 22), 28 contiguous amino acid residues at or near the N-terminus of the wild-type NiV-G protein (SEQ ID NO: 22), 29 contiguous amino acid residues at or near the N-terminus of the wild-type NiV-G protein (SEQ ID NO: 22), up to 30 contiguous amino acid residues at or near the N-terminus of the wild-type NiV-G protein (EQ ID NO: 22), up to 31 contiguous amino acid residues at or near the N-terminus of the wild-type NiV-G protein (SEQ ID NO: 22), 32 contiguous amino acid residues at or near the N-terminus of the wild-type NiV-G protein (SEQ ID NO: 22), 33 contiguous amino acid residues at or near the N-terminus of the wild-type NiV-G protein (SEQ ID NO: 22) 34 contiguous amino acid residues at or near the N-terminus of the wild-type NiV-G protein (SEQ ID NO: 22), 35 contiguous amino acid residues at or near the N-terminus of the wild-type NiV-G protein (SEQ ID NO: 22) up to 36 contiguous amino acid residues at or near the N-terminus of the wild-type NiV-G protein (EQ ID NO: 22), up to 37 contiguous amino acid residues at or near the N-terminus of the wild-type NiV-G protein (EQ ID NO: 22), up to 38 contiguous amino acid residues at or near the N-terminus of the wild-type NiV-G protein (EQ ID NO: 22), up to 39 contiguous amino acid residues at or near the N-terminus of the wild-type NiV-G protein (SEQ ID NO: 22), or up to 40 contiguous amino acid residues at or near the N-terminus of the wild-type NiV-G protein (EQ ID NO: 22).

In some embodiments, the mutant NiV-G protein has the amino acid sequence set forth in SEQ ID NO: 35 or 36 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 35 or 36. In particular embodiments, the G protein has the sequence of amino acids set forth in SEQ ID NO 35 or 36.

In some embodiments, the G protein is a mutant G protein containing one or more amino acid substitutions selected from the group consisting of E501A, W504A, Q530A and E533A with reference to numbering set forth in SEQ ID NO:24. In some embodiments, the G protein is a mutant G protein that contains one or more amino acid substitutions elected from the group consisting of E501A, W504A, Q530A and E533A with reference to SEQ ID NO:24 and is a biologically active portion thereof containing an N-terminal truncation.

b. F Proteins

In some embodiments, the vector-surface targeting moiety comprises a protein with a hydrophobic fusion peptide domain. In some embodiments, the vector-surface targeting moiety comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein or a bat Paramyxovirus F protein or a biologically active portion thereof.

Table 2 provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F₀ precursors containing a signal peptide (e.g. corresponding to amino acid residues 1-26 of SEQ ID NO:46). Following cleavage of the signal peptide, the mature F₀ (e.g. SEQ ID NO:47) is transported to the cell surface, then endocytosed and cleaved by cathepsin L into the mature fusogenic subunits F1 and F2. The F1 and F2 subunits are associated by a disulfide bond and recycled back to the cell surface. The F1 subunit contains the fusion peptide domain located at the N terminus of the F1 subunit, where it is able to insert into a cell membrane to drive fusion. In some aspects, fusion is blocked by association of the F protein with G protein, until the G protein engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects or the provided re-targeted lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. Journal of Virology. 2019. 93(13):e00577-19). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 2
F proteins

| | | | SEQ ID |
|---|---|---|---|
| | | | (without |
| Full Gene | | SEQ | signal |
| Name | Sequence | ID | sequence) |
| Hendra virus | MATQEVRLKCLLCGIIVLVL | 46 | 47 |
| F Protein | SLEGLGILHYEKLSKIGLVK | | |
| | GITRKYKIKSNPLTKDIVIK | | |
| | MIPNVSNVSKCTGTVMENYK | | |
| | SRLTGILSPIKGAIELYNNN | | |
| | THDLVGDVKLAGVVMAGIAI | | |
| | GIATAAQITAGVALYEAMKN | | |
| | ADNINKLKSSIESTNEAVVK | | |
| | LQETAEKTVYVLTALQDYIN | | |
| | TNLVPTIDQISCKQTELALD | | |
| | LALSKYLSDLLFVFGPNLQD | | |
| | PVSNSMTIQAISQAFGGNYE | | |
| | TLLRTLGYATEDFDDLLESD | | |
| | SIAGQIVYVDLSSYYIIVRV | | |
| | YFPILTEIQQAYVQELLPVS | | |
| | FNNDNSEWISIVPNFVLIRN | | |
| | TLISNIEVKYCLITKKSVIC | | |
| | NQDYATPMTASVRECLTGST | | |
| | DKCPRELVVSSHVPRFALSG | | |
| | GVLFANCISVTCQCQTTGRA | | |
| | ISQSGEQTLLMIDNTTCTTV | | |
| | VLGNIIISLGKYLGSINYNS | | |
| | ESIAVGPPVYTDKVDISSQI | | |
| | SSMNQSLQQSKDYIKEAQKI | | |
| | LDTVNPSLISMLSMIILYVL | | |
| | SIAALCIGLITFISFVIVEK | | |
| | KRGNYSRLDDRQVRPVSNGD | | |
| | LYYIGT | | |
| Nipah virus F | MVVILDKRCYCNLLILILMI | 48 | 49 |
| Protein | SECSVGILHYEKLSKIGLVK | | |
| | GVTRKYKIKSNPLTKDIVIK | | |
| | MIPNVSNMSQCTGSVMENYK | | |
| | TRLNGILTPIKGALEIYKNN | | |
| | THDLVGDVRLAGVIMAGVAI | | |
| | GIATAAQITAGVALYEAMKN | | |
| | ADNINKLKSSIESTNEAVVK | | |
| | LQETAEKTVYVLTALQDYIN | | |
| | TNLVPTIDKISCKQTELSLD | | |
| | LALSKYLSDLLFVFGPNLQD | | |
| | PVSNSMTIQAISQAFGGNYE | | |
| | TLLRTLGYATEDFDDLLESD | | |
| | SITGQIIYVDLSSYYIIVRV | | |
| | YFPILTEIQQAYIQELLPVS | | |
| | FNNDNSEWISIVPNFILVRN | | |
| | TLISNIEIGFCLITKRSVIC | | |
| | NQDYATPMTNNMRECLTGST | | |
| | EKCPRELVVSSHVPRFALSN | | |
| | GVLFANCISVTCQCQTTGRA | | |
| | ISQSGEQTLLMIDNTTCPTA | | |
| | VLGNVIISLGKYLGSVNYNS | | |
| | EGIAIGPPVFTDKVDISSQI | | |
| | SSMNQSLQQSKDYIKEAQRL | | |
| | LDTVNPSLISMLSMIILYVL | | |
| | SIASLCIGLITFISFIIVEK | | |
| | KRNTYSRLEDRRVRPTSSGD | | |
| | LYYIGT | | |
| Cedar Virus F | MSNKRTTVLIIISYTLFYLN | 50 | 51 |
| Protein | NAAIVGFDFDKLNKIGVVQG | | |
| | RVLNYKIKGDPMTKDLVLKF | | |
| | IPNIVNITECVREPLSRYNE | | |
| | TVRRLLLPIHNMLGLYLNNT | | |
| | NAKMTGLMIAGVIMGGIAIG | | |
| | IATAAQITAGFALYEAKKNT | | |
| | ENIQKLTDSIMKTQDSIDKL | | |
| | TDSVGTSILILNKLQTYINN | | |
| | QLVPNLELLSCRQNKIEFDL | | |
| | MLTKYLVDLMTVIGPNINNP | | |
| | VNKDMTIQSLSLLFDGNYDI | | |
| | MMSELGYTPQDFLDLIESKS | | |
| | ITGQIIYVDMENLYVVIRTY | | |
| | LPTLIEVPDAQIYEFNKITM | | |
| | SSNGGEYLSTIPNFILIRGN | | |
| | YMSNIDVATCYMTKASVICN | | |
| | QDYSLPMSQNLRSCYQGETE | | |
| | YCPVEAVIASHSPRFALTNG | | |
| | VIFANCINTICRCQDNGKTI | | |
| | TQNINQFVSMIDNSTCNDVM | | |
| | VDKFTIKVGKYMGRKDINNI | | |
| | NIQIGPQIIIDKVDLSNEIN | | |
| | KMNQSLKDSIFYLREAKRIL | | |
| | DSVNISLISPSVQLFLIIIS | | |
| | VLSFIILLIIIVYLYCKSKH | | |
| | SYKYNKFIDDPDYYNDYKRE | | |
| | RINGKASKSNNIYYVGD | | |
| Mojiang virus, | MALNKNMFSSLFLGYLLVYA | 52 | 53 |
| Tongguan 1 F | TTVQSSIHYDSLSKVGVIKG | | |
| Protein | LTYNYKIKGSPSTKLMVVKL | | |
| | IPNIDSVKNCTQKQYDEYKN | | |
| | LVRKALEPVKMAIDTMLNNV | | |
| | KSGNNKYRFAGAIMAGVALG | | |
| | VATAATVTAGIALHRSNENA | | |
| | QAIANMKSAIQNTNEAVKQL | | |
| | QLANKQTLAVIDTIRGEINN | | |
| | NIIPVINQLSCDTIGLSVGI | | |
| | RLTQYYSEIITAFGPALQNP | | |
| | VNTRITIQAISSVFNGNFDE | | |
| | LLKIMGYTSGDLYEILHSEL | | |
| | IRGNIIDVDVDAGYIALEIE | | |
| | FPNLTLVPNAVVQELMPISY | | |
| | NIDGDEWVTLVPRFVLTRTT | | |
| | LLSNIDTSRCTITDSSVICD | | |
| | NDYALPMSHELIGCLQGDTS | | |
| | KCAREKVVSSYVPKFALSDG | | |
| | LVYANCLNTICRCMDTDTPI | | |
| | SQSLGATVSLLDNKRCSVYQ | | |
| | VGDVLISVGSYLGDGEYNAD | | |
| | NVELGPPIVIDKIDIGNQLA | | |
| | GINQTLQEAEDYIEKSEEFL | | |
| | KGVNPSIITLGSMVVLYIFM | | |
| | ILIAIVSVIALVLSIKLTVK | | |
| | GNVVRQQFTYTQHVPSMENI | | |
| | NYVSH | | |
| Bat | MKKKTDNPTISKRGHNHSRG | 54 | 55 |
| Paramyxovirus | IKSRALLRETDNYSNGLIVE | | |
| Eid_hel/GH- | NLVRNCHHPSKNNLNYTKTQ | | |
| M74a/GHA/2 | KRDSTIPYRVEERKGHYPKI | | |
| 009 F protein | KHLIDKSYKHIKRGKRRNGH | | |
| | NGNIITIILLLILILKTQMS | | |
| | EGAIHYETLSKIGLIKGITR | | |
| | EYKVKGTPSSKDIVIKLIPN | | |
| | VTGLNKCTNISMENYKEQLD | | |
| | KILIPINNIIELYANSTKSA | | |
| | PGNARFAGVIIAGVALGVAA | | |
| | AAQITAGIALHEARQNAERI | | |
| | NLLKDSISATNNAVAELQEA | | |
| | TGGIVNVITGMQDYINTNLV | | |
| | PQIDKLQCSQIKTALDISLS | | |
| | QYYSEILTVFGPNLQNPVTT | | |
| | SMSIQAISQSFGGNIDLLLN | | |
| | LLGYTANDLLDLLESKSITG | | |
| | QITYINLEHYFMVIRVYYPI | | |
| | MTTISNAYVQELIKISFNVD | | |
| | GSEWVSLVPSYILIRNSYLS | | |
| | NIDISECLITKNSVICRHDF | | |
| | AMPMSYTLKECLTGDTEKCP | | |
| | REAVVTSYVPRFAISGGVIY | | |
| | ANCLSTTCQCYQTGKVIAQD | | |
| | GSQTLMMIDNQTCSIVRIEE | | |
| | ILISTGKYLGSQEYNTMHVS | | |
| | VGNPVFTDKLDITSQISNIN | | |
| | QSIEQSKFYLDKSKAILDKI | | |
| | NLNLIGSVPISILFIIAILS | | |
| | LILSIITFVIVMIIVRRYNK | | |
| | YTPLINSDPSSRRSTIQDVY | | |
| | IIPNPGEHSIRSAARSIDRD | | |
| | RD | | |

In some embodiments, the F protein is encoded by a nucleotide sequence that encodes the sequence set forth by any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25 or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55.

In particular embodiments, the F protein or the functionally active variant or biologically active portion thereof retains fusogenic activity in conjunction with a Henipavirus G protein, such as a G protein set forth in Section IV.A.2 (e.g. NiV-G or HeV-G). Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:48).

In particular embodiments, the F protein has the sequence of amino acids set forth in SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55, and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55.

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55,

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 46-55. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F₀ precursor is encoded by a sequence of nucleotides encoding the sequence set forth in SEQ ID NO: 37. The encoding nucleic acid can encode a signal peptide sequence that has the sequence MVVILDKRCY CNLLILILMI SECSVG (SEQ ID NO: 56). In some embodiments, the F protein has the sequence set forth in SEQ ID NO:18. In some examples, the F protein is cleaved into an F1 subunit comprising the sequence set forth in SEQ ID NO:57 and an F2 subunit comprising the sequence set forth in SEQ ID NO: 58.

In some embodiments, the F protein is a NiV-F protein that is encoded by a sequence of nucleotides encoding the sequence set forth in SEQ ID NO:48, or is a functionally active variant or biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 48. In some embodiments, the NiV-F-protein has the sequence of set forth in SEQ ID NO: 25, or is a functionally active variant or a biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 25. In particular embodiments, the F protein or the functionally active variant or biologically active portion thereof retains the cleavage site cleaved by cathepsin L.

In some embodiments, the F protein or the functionally active variant or the biologically active portion thereof includes an F1 subunit that has the sequence set forth in SEQ ID NO: 57, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:57.

In some embodiments, the F protein or the functionally active variant or biologically active portion thereof includes an F2 subunit that has the sequence set forth in SEQ ID NO: 58, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:58.

In some embodiments, the F protein or the functionally active variant or the biologically active portion thereof includes an F1 subunit that has the sequence set forth in SEQ ID NO: 23, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:23.

In some embodiments, the F protein or the functionally active variant or biologically active portion thereof includes an F2 subunit that has the sequence set forth in SEQ ID NO: 24, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:24.

In some embodiments, the F protein is a mutant NiV-F protein that is a biologically active portion thereof that is truncated and lacks up to 20 contiguous amino acid residues at or near the C-terminus of the wild-type NiV-F protein (e.g. set forth SEQ ID NO:57). In some embodiments, the mutant NiV-F protein comprises an amino acid sequence set forth in SEQ ID NO:25. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 25. In some embodiments, the mutant F protein contains an F1 protein that has the sequence set forth in SEQ ID NO:26. In some embodiments, the mutant F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 26.

In some embodiments, the F protein is a mutant NiV-F protein that is a biologically active portion thereof that comprises a 20 amino acid truncation at or near the C-terminus of the wild-type NiV-F protein (SEQ ID NO:57); and a point mutation on an N-linked glycosylation site. In some embodiments, the mutant NiV-F protein comprises an amino acid sequence set forth in SEQ ID NO: 27. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 27.

In some embodiments, the F protein is a mutant NiV-F protein that is a biologically active portion thereof that comprises a 22 amino acid truncation at or near the C-terminus of the wild-type NiV-F protein (SEQ ID NO:57). In some embodiments, the NiV-F protein is encoded by a nucleotide sequence that encodes the sequence set forth in SEQ ID NO: 28. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 28.

B. Non-protein Fusogens

In some embodiments, the non-cell particle can comprise one or more fusogenic lipids, such as saturated fatty acids. In some embodiments, the saturated fatty acids have between 10-14 carbons. In some embodiments, the saturated fatty acids have longer-chain carboxylic acids. In some embodiments, the saturated fatty acids are mono-esters.

In some embodiments, the non-cell particle can comprise one or more unsaturated fatty acids. In some embodiments, the unsaturated fatty acids have between C16 and C18 unsaturated fatty acids. In some embodiments, the unsaturated fatty acids include oleic acid, glycerol mono-oleate, glycerides, diacylglycerol, modified unsaturated fatty acids, and any combination thereof.

Without wishing to be bound by theory, in some embodiments negative curvature lipids promote membrane fusion. In some embodiments, the non-cell particle comprises one or more negative curvature lipids, e.g., exogenous negative curvature lipids, in the membrane. In embodiments, the negative curvature lipid or a precursor thereof is added to media comprising source cells or non-cell particles. In embodiments, the source cell is engineered to express or overexpress one or more lipid synthesis genes. The negative curvature lipid can be, e.g., diacylglycerol (DAG), cholesterol, phosphatidic acid (PA), phosphatidylethanolamine (PE), or fatty acid (FA).

In some embodiments positive curvature lipids inhibit membrane fusion. In some embodiments, the non-cell particle comprises reduced levels of one or more positive curvature lipids, e.g., exogenous positive curvature lipids, in the membrane. In embodiments, the levels are reduced by inhibiting synthesis of the lipid, e.g., by knockout or knockdown of a lipid synthesis gene, in the source cell. The positive curvature lipid can be, e.g., lysophosphatidylcholine (LPC), phosphatidylinositol (Ptdlns), lysophosphatidic acid (LPA), lysophosphatidylethanolamine (LPE), or monoacylglycerol (MAG).

In some embodiments, the non-cell particle may be treated with fusogenic chemicals.

In some embodiments, the fusogenic chemical is polyethylene glycol (PEG) or derivatives thereof.

In some embodiments, the chemical fusogen induces a local dehydration between the two membranes that leads to unfavorable molecular packing of the bilayer. In some embodiments, the chemical fusogen induces dehydration of an area near the lipid bilayer, causing displacement of aqueous molecules between two membranes and allowing interaction between the two membranes together.

In some embodiments, the chemical fusogen is a positive cation. Some nonlimiting examples of positive cations include Ca2+, Mg2+, Mn2+, Zn2+, La3+, Sr3+, and H+.

In some embodiments, the chemical fusogen binds to the target membrane by modifying surface polarity, which alters the hydration-dependent intermembrane repulsion.

In some embodiments, the chemical fusogen is a soluble lipid soluble. Some nonlimiting examples include oleoylglycerol, dioleoylglycerol, trioleoylglycerol, and variants and derivatives thereof.

In some embodiments, the chemical fusogen is a water-soluble chemical. Some nonlimiting examples include polyethylene glycol, dimethyl sulphoxide, and variants and derivatives thereof.

In some embodiments, the chemical fusogen is a small organic molecule. A nonlimiting example includes n-hexyl bromide.

In some embodiments, the chemical fusogen does not alter the constitution, cell viability, or the ion transport properties of the fusogen or target membrane.

In some embodiments, the chemical fusogen is a hormone or a vitamin. Some nonlimiting examples include abscisic acid, retinol (vitamin A1), a tocopherol (vitamin E), and variants and derivatives thereof.

In some embodiments, the retroviral vector or VLP comprises actin and an agent that stabilizes polymerized actin. Without wishing to be bound by theory, stabilized actin in a retroviral vector or VLP can promote fusion with a target cell. In embodiments, the agent that stabilizes polymerized actin is chosen from actin, myosin, biotin-streptavidin, ATP, neuronal Wiskott-Aldrich syndrome protein (N-WASP), or formin. See, e.g., Langmuir. 2011 Aug. 16; 27(16):10061-71 and Wen et al., Nat Commun. 2016 Aug. 31;7. In embodiments, the retroviral vector or VLP comprises exogenous actin, e.g., wild-type actin or actin comprising a mutation that promotes polymerization. In embodiments, the retroviral vector or VLP comprises ATP or phosphocreatine, e.g., exogenous ATP or phosphocreatine.

In some embodiments, the non-cell particle may be treated with fusogenic small molecules. Some nonlimiting examples include halothane, nonsteroidal anti-inflammatory drugs (NSAIDs) such as meloxicam, piroxicam, tenoxicam, and chlorpromazine.

In some embodiments, the small molecule fusogen may be present in micelle-like aggregates or free of aggregates.

C. Re-Targeted Fusogens

In some embodiments, protein fusogens or viral envelope proteins may be re-targeted by mutating amino acid residues in a fusion protein or a targeting protein (e.g. the hemagglutinin protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the non-cell particle. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a targeting-moiety to the fusion protein or targeting protein (e.g. the hemagglutinin protein). In some embodiments, the fusogen and targeting moiety are covalently conjugated by expression of a chimeric protein comprising the fusogen linked to the targeting moiety. In some embodiments, a target includes any peptide (e.g. a receptor) that is displayed on a target cell. In some embodiments, the target is expressed at higher levels on a target cell than non-target cells. In some embodiments, a single-chain variable fragment (scFv) can be conjugated to fusogens to redirect fusion activity towards cells that display the scFv binding target (doi:10.1038/nbtl060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a targeting moiety to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a targeting moiety comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies@); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein.

In some embodiments, vector-surface targeting moiety is a peptide. In some embodiments, vector-surface targeting moiety is an antibody, such as a single domain antibody.

In some embodiments, the antibody can be human or humanized. In some embodiments, antibody or portion thereof is naturally occurring. In some embodiments, the antibody or portion thereof is synthetic.

In some embodiments, the antibody can be generated from phage display libraries to have specificity for a desired target ligand. In some embodiments the target ligand is expressed in the lung, such as ACE2. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the vector-surface targeting moiety is attached to the C-terminus of the G protein (e.g., fusogen) or biologically active portion thereof. In some embodiments, the N-terminus of the vector-surface targeting moiety is exposed on the exterior surface of the lipid bilayer. In some embodiments, the N-terminus of the vector-surface targeting moiety binds to a cell surface molecule of a target cell. In some embodiments, the vector-surface targeting moiety specifically binds to a cell surface molecule present on a target cell. In some embodiments, the vector-surface targeting moiety is a protein, glycan, lipid or low molecular weight molecule.

In some embodiments, the cell surface marker is a molecule expressed on a target cell that is an antigen or portion thereof recognized by the targeting moeity.

Exemplary target cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes,

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cadiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoeietic stem cell (HSC), a liver cell or a fully differentiated cell. In some embodiments, the target cell is a CD3+T cell, a CD4+ Tcell, a CD8+T cell, a hepatocyte, a haematepoietic stem cell, a CD34+ haematepoietic stem cell, a CD105+ haematepoietic stem cell, a CD117+ haematepoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell).

In some embodiments, the cell surface marker is any one of CD8, CD4, asialoglycoprotein receptor 2 (ASGR2), transmembrane 4 L6 family member 5 (TM4SF5), low density lipoprotein receptor (LDLR) or asialoglycoprotein 1 (ASGR1).

In some embodiments, non-cell particles may display targeting moieties that are not conjugated to protein fusogens in order to redirect the fusion activity towards a cell that is bound by the targeting moiety, or to affect homing.

In some embodiments, the targeting moiety added to the non-cell particle is modulated to have different binding strengths. In some embodiments, scFvs and antibodies with various binding strengths may be used to alter the fusion activity of the non-cell particle towards cells that display high or low amounts of the target antigen (doi:10.1128/JVI.01415-07, doi:10.1038/cgt.2014.25, DOI: 10.1002/jgm.1151). In some embodiments, DARPins with different affinities may be used to alter the fusion activity of the non-cell particle towards cells that display high or low amounts of the target antigen (doi:10.1038/mt.2010.298). In some embodiments, targeting moieties may also be modulated to target different regions on the target ligand, which will affect the fusion rate with cells displaying the target (doi: 10.1093/protein/gzv005).

In some embodiments protein fusogens can be altered to reduce immunoreactivity, e.g., as described herein. In some embodiments, protein fusogens may be decorated with molecules that reduce immune interactions, such as PEG (DOI: 10.1128/JVI.78.2.912-921.2004). In some embodiments, the fusogen comprises PEG, e.g., is a PEGylated polypeptide.

In some embodiments, amino acid residues in the fusogen that are targeted by the immune system may be altered to be unrecognized by the immune system (doi: 10.1016/j.virol.2014.01.027, doi:10.1371/journal.pone.0046667). In some embodiments the protein sequence of the fusogen is altered to resemble amino acid sequences found in humans (humanized). In some embodiments the protein sequence of the fusogen is changed to a protein sequence that binds MHC complexes less strongly. In some embodiments, the protein fusogens are derived from viruses or organisms that do not infect humans (and which humans have not been vaccinated against), increasing the likelihood that a patient's immune system is naive to the protein fusogens (e.g., there is a negligible humoral or cell-mediated adaptive immune response towards the fusogen) (doi:10.1006/mthe.2002.0550, doi:10.1371/journal.ppat.1005641, doi:10.1038/gt.2011.209, DOI 10.1182/blood-2014-02-558163). In some embodiments, glycosylation of the fusogen may be changed to alter immune interactions or reduce immunoreactivity.

In some embodiments, a protein fusogen derived from a virus or organism that do not infect humans does not have a natural fusion targets in patients, and thus has high specificity.

### V. EXOGENOUS AGENT

In some embodiments, the non-cell particle or pharmaceutical composition comprising same described herein contains an exogenous agent. In some embodiments, the non-cell particle or pharmaceutical composition comprising same described herein contains a nucleic acid that encodes an exogenous agent. In some embodiments, the non-cell particle contains the exogenous agent. In some embodiments, the non-cell particle contains a nucleic acid that encodes an exogenous agent. Reference to the coding sequence of the nucleic acid encoding the exogenous agent also is referred to herein as a payload gene. In some embodiments, the exogenous agent or the nucleic acid encoding the exogenous agent are present in the lumen of the non-cell particle.

In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent comprises or encodes a membrane protein. In some embodiments, the exogenous agent comprises or encodes a therapeutic agent. In some embodiments, the therapeutic agent is chosen from one or more of a protein, e.g., an enzyme, a transmembrane protein, a receptor, or an antibody; a nucleic acid, e.g., DNA, a chromosome (e.g. a human artificial chromosome), RNA, mRNA, siRNA, or miRNA; or a small molecule.

In some embodiments, the non-cell particle or pharmaceutical composition delivers to a target cell at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the non-cell particle. In some embodiments, the non-cell particle, e.g., fusosome, that contacts, e.g., fuses, with the target cell(s) delivers to the target cell an average of at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the non-cell particles, e.g., fusosomes, that contact, e.g., fuse, with the target cell(s). In some embodiments, the non-cell particle composition delivers to a target tissue at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the non-cell particle compositions.

In some embodiments, the exogenous agent is not expressed naturally in the cell from which the non-cell particle is derived. In some embodiments, the exogenous agent is expressed naturally in the cell from which the non-cell particle is derived. In some embodiments, the exogenous agent is loaded into the non-cell particle via expression in the cell from which the non-cell particle is derived (e.g. expression from DNA or mRNA introduced via transfection, transduction, or electroporation). In some embodiments, the exogenous is expressed from DNA integrated into the genome or maintained episosomally. In some embodiments, expression of the exogenous agent is constitutive. In some embodiments, expression of the exogenous agent is induced. In some embodiments, expression of the exogenous agent is induced immediately prior to generating the non-cell particle. In some embodiments, expression of the exogenous agent is induced at the same time as expression of the fusogen.

In some embodiments, the exogenous agent is loaded into the non-cell particle via electroporation into the non-cell particle itself or into the cell from which the non-cell particle is derived. In some embodiments, the exogenous agent is loaded into the non-cell particle via transfection (e.g., of a DNA or mRNA encoding the exogenous agent) into the non-cell particle itself or into the cell from which the non-cell particle is derived.

In some embodiments, the exogenous agent may include one or more nucleic acid sequences, one or more polypeptides, a combination of nucleic acid sequences and/or polypeptides, one or more organelles, and any combination thereof. In some embodiments, the exogenous agent may include one or more cellular components. In some embodiments, the exogenous agent includes one or more cytosolic and/or nuclear components.

In some embodiments, the non-cell particle contains an exogenous agent that is a nucleic acid or contains a nucleic acid encoding the exogenous agent. In some embodiments, the nucleic acid is operatively linked to a “positive target cell-specific regulatory element” (or positive TCSRE). In some embodiments, the positive TCSRE is a functional nucleic acid sequence. In some embodiments, the positive TCSRE comprises a promoter or enhancer. In some embodiments, the TCSRE is a nucleic acid sequence that increases the level of an exogenous agent in a target cell. In some embodiments, the positive target cell-specific regulatory element comprises a T cell-specific promoter, a T cell-specific enhancer, a T cell-specific splice site, a T cell-specific site extending half-life of an RNA or protein, a T cell-specific mRNA nuclear export promoting site, a T cell-specific translational enhancing site, or a T cell-specific post-translational modification site. In some embodiments, the T cell-specific promoter is a promoter described in Immgen consortium, herein incorporated by reference in its entirety, e.g., the T cell-specific promoter is an IL2RA (CD25), LRRC32, FOXP3, or IKZF2 promoter. In some embodiments, the T cell-specific promoter or enhancer is a promoter or enhancer described in Schmidl et a, Blood. 2014 Apr. 24;123(17):e68-78., herein incorporated by reference in its entirety. In some embodiments, the T cell-specific promoter is a transcriptionally active fragment of any of the foregoing. In some embodiments, the T-cell specific promoter is a variant having at least 70%, 75%, 80%, 85%, 90%, 95%, 96%, 97%, 98%, or 99% identity to any of the foregoing.

In some embodiments, the non-cell particle contains an exogenous agent that is a nucleic acid or contains a nucleic acid encoding the exogenous agent. In some embodiments, the nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the non-cell particle in a non-target cell. In some embodiments, the exogenous agent is operatively linked to a “non-target cell-specific regulatory element” (or NTCSRE). In some embodiments, the NTCSRE comprises a nucleic acid sequence that decreases the level of an exogenous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR3 1, miR363, or miR29c. In some embodiments, the NTCSRE is situated or encoded within a transcribed region encoding the exogenous agent, optionally wherein an RNA produced by the transcribed region comprises the miRNA recognition sequence within a UTR or coding region.

A. Nucleic Acids

In some embodiments, the exogenous agent may include a nucleic acid. For example, the exogenous agent may comprise RNA to enhance expression of an endogenous protein, or a siRNA or miRNA that inhibits protein expression of an endogenous protein. For example, the endogenous protein may modulate structure or function in the target cells. In some embodiments, the exogenous agent may include a nucleic acid encoding an engineered protein that modulates structure or function in the target cells. In some embodiments, the exogenous agent is a nucleic acid that targets a transcriptional activator that modulate structure or function in the target cells

In some embodiments, a non-cell particle described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide. The nucleic acid may include variants, e.g., having an overall sequence identity with a reference nucleic acid of at least 85%, 86%, 87%, 88%, 89%, 90%, 91%, 92%, 93%, 94%, 95%, 96%, 97%, or 99%. In some embodiments, a variant nucleic acid does not share at least one characteristic sequence element with a reference nucleic acid. In some embodiments, a variant nucleic acid shares one or more of the biological activities of the reference nucleic acid. In some embodiments, a nucleic acid variant has a nucleic acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant nucleic acid comprises about 10, about 9, about 8, about 7, about 6, about 5, about 4, about 3, about 2, or about 1 substituted residue as compared to a reference. In some embodiments, a variant nucleic acid comprises a very small number (e.g., fewer than about 5, about 4, about 3, about 2, or about 1) number of substituted, inserted, or deleted, functional residues that participate in a particular biological activity relative to the reference. In some embodiments, a variant nucleic acid comprises not more than about 15, about 12, about 9, about 3, or about 1 addition or deletion, and, in some embodiments, comprises no additions or deletions, as compared to the reference. In some embodiments, a variant nucleic acid comprises fewer than about 27, about 24, about 21, about 18, about 15, about 12, about 9, about 6, about 3, or fewer than about 9, about 6, about 3, or about 2 additions or deletions as compared to the reference.

In some embodiments, the exogenous agent includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences

In embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA.

In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155. In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target. In some embodiments, the exogenous agent comprises a shRNA. A shRNA (short hairpin RNA) can comprise a double-stranded structure that is formed by a single self complementary RNA strand. shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 2 1 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H 1 RNA promoter and the human tRNA-val promoter, or a strong constitutive pol II promoter.

B. Polypeptides

In some embodiments, the non-cell particle contains a nucleic acid that encodes a protein exogenous agent (also referred to as a “payload gene encoding an exogenous agent.”). In some embodiments, a non-cell particle described herein comprises an exogenous agent which is or comprises a protein.

In some embodiments, the protein may include moieties other than amino acids (e.g., may be glycoproteins, proteoglycans, etc.) and/or may be otherwise processed or modified. In some embodiments, the protein can sometimes include more than one polypeptide chain, for example linked by one or more disulfide bonds or associated by other means.

In some embodiments, the protein may contain L-amino acids, D-amino acids, or both and may contain any of a variety of amino acid modifications or analogs. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof. In some embodiments, a polypeptide may include its variants, e.g., having an overall sequence identity with a reference polypeptide of at least 85%, 86%, 87%, 88%, 89%, 90%, 91%, 92%, 93%, 94%, 95%, 96%, 97%, or 99%. In some embodiments, a variant polypeptide does not share at least one characteristic sequence element with a reference polypeptide. In some embodiments, a variant polypeptide shares one or more of the biological activities of the reference polypeptide. In some embodiments, a polypeptide variant has an amino acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide comprises about 10, about 9, about 8, about 7, about 6, about 5, about 4, about 3, about 2, or about 1 substituted residue as compared to a reference. In some embodiments, a variant polypeptide comprises a very small number (e.g., fewer than about 5, about 4, about 3, about 2, or about 1) number of substituted, inserted, or deleted, functional that participate in a particular biological activity relative to the reference. In some embodiments, a variant polypeptide comprises not more than about 5, about 4, about 3, about 2, or about 1 addition or deletion, and, in some embodiments, comprises no additions or deletions, as compared to the reference. In some embodiments, a variant polypeptide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments, the protein targets a protein in the cell for degradation. In some embodiments, the protein targets a protein in the cell for degradation by localizing the protein to the proteasome. In some embodiments, the protein is a wild-type protein. In some embodiments, the protein is a mutant protein.

Exemplary protein exogenous agents are described in the following subsections. In some embodiments, a non-cell particle provided herein can include any of such exogenous agents. In particular embodiments, a non-cell particle contains a nucleic acid encoding any of such exogenous agents.

a. Cytosolic Proteins

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

b. Membrane Proteins

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

1) Chimeric Antigen Receptors (CARs)

In some embodiments, a payload gene described herein encodes a chimeric antigen receptor (CAR) comprising an antigen binding domain. In some embodiments, an exogenous agent described herein comprises a chimeric antigen receptor (CAR) comprising an antigen binding domain. In some embodiments, the payload is or comprises a chimeric antigen receptor (CAR) comprising an antigen binding domain. In some embodiments, the CAR is or comprises a first generation CAR comprising an antigen binding domain, a transmembrane domain, and signaling domain (e.g., one, two or three signaling domains). In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, the antigen binding domain targets an antigen characteristic of a cell type. In some embodiments, the antigen binding domain targets an antigen characteristic of a neoplastic cell. In some embodiments, the antigen characteristic of a neoplastic cell is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA-4; GITR; GARP; LAP; granzyme B; LFA-1; transferrin receptor; NKp46, perforin, CD4+; 0; Th2; Th17; Th40; Th22; Th9; Tfh, Canonical Treg. FoxP3+; Tr1; Th3; Treg17; T_{RE}G; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-10, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), LiCAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACl, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p⁵³ mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RUi, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

In some embodiments, the antigen binding domain targets an antigen characteristic of a T cell. In some embodiments, the antigen characteristic of a T cell is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD36); CD3E (CD3R); CD3G (CD37); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments, the antigen binding domain targets an antigen characteristic of a disorder. In some embodiments, the antigen binding domain targets an antigen characteristic of an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor. In some embodiments, a CAR antigen binding domain binds to a ligand expressed on B cells, plasma cells, plasmablasts, CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See US 2003/0077249; WO 2017/058753; WO 2017/058850, the contents of which are herein incorporated by reference.

In some embodiments, the antigen binding domain targets an antigen characteristic of senescent cells, e.g., urokinase-type plasminogen activator receptor (uPAR). In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Eptstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gpl20, or CD4-induced epitope on HIV-1 Env.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FcERIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

In some embodiments, the CAR comprises at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets.

In some embodiments the exogenous agent is or comprises a CAR, e.g., a first generation CAR or a nucleic acid encoding a first generation CAR. In some embodiments, a first generation CAR comprises an antigen binding domain, a transmembrane domain, and signaling domain. In some embodiments a signaling domain mediates downstream signaling during T cell activation.

In some embodiments the exogenous agent is or comprises a second generation CAR or a nucleic acid encoding a second generation CAR. In some embodiments a second generation CAR comprises an antigen binding domain, a transmembrane domain, and two signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some embodiments the exogenous agent is or comprises a third generation CAR or a nucleic acid encoding a third generation CAR. In some embodiments, a third generation CAR comprises an antigen binding domain, a transmembrane domain, and at least three signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments the exogenous is or comprises a fourth generation CAR or a nucleic acid encoding a fourth generation CAR. In some embodiments a fourth generation CAR comprises an antigen binding domain, a transmembrane domain, and at least two, three, or four signaling domains. In some embodiments a signaling domain mediates downstream signaling during T cell activation. In some embodiments a signaling domain is a costimulatory domain.

In some embodiments, a costimulatory domain enhances cytokine production, CAR T cell proliferation, and or CAR T cell persistence during T cell activation.

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments a cytokine gene encodes a pro-inflammatory cytokine. In some embodiments a cytokine gene encodes IL-1, IL-2, IL-9, IL-12, IL-18, TNF, or IFN-gamma, or functional fragment thereof. In some embodiments a domain which upon successful signaling of the CAR induces expression of a cytokine gene is or comprises a transcription factor or functional domain or fragment thereof. In some embodiments a domain which upon successful signaling of the CAR induces expression of a cytokine gene is or comprises a transcription factor or functional domain or fragment thereof. In some embodiments a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, a CAR antigen binding domain is or comprises an antibody or antigen-binding portion thereof. In some embodiments, a CAR antigen binding domain is or comprises an scFv or Fab. In some embodiments a CAR antigen binding domain comprises an scFv or Fab fragment of a T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, an antigen binding domain binds to a cell surface antigen of a cell. In some embodiments, a cell surface antigen is characteristic of one type of cell. In some embodiments, a cell surface antigen is characteristic of more than one type of cell.

In some embodiments a CAR antigen binding domain binds a cell surface antigen characteristic of a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen characteristic of a T cell may be a T cell receptor. In some embodiments, a T cell receptor may be AKT1; AKT2; AKT3; ATF2; BCL10; CALM1; CD3D (CD36); CD3E (CD3F); CD3G (CD37); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p³⁸J); MAPK12 (p³⁸γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments a CAR comprises a signaling domain which is a costimulatory domain. In some embodiments a CAR comprises a second costimulatory domain. In some embodiments a CAR comprises at least two costimulatory domains. In some embodiments a CAR comprises at least three costimulatory domains. In some embodiments a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

In some embodiments a non-cell particle comprising a CAR or a nucleic acid encoding a CAR (e.g., a DNA, a gDNA, a cDNA, an RNA, a pre-MRNA, an mRNA, an miRNA, an siRNA, etc.) is delivered to a target cell. In some embodiments the target cell is an effector cell, e.g., a cell of the immune system that expresses one or more Fc receptors and mediates one or more effector functions. In some embodiments, a target cell may include, but may not be limited to, one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including but not limited to humans, mice, rats, rabbits, and monkeys.

C. Small Molecules

In some embodiments, the exogenous agent includes a small molecule, e.g., ions (e.g. Ca²⁺, C1-, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

### VI. EXEMPLARY FEATURES OF CD24 ASSOCIATED NON-CELL PARTICLES

In some embodiments of any of the aspects described herein, the non-cell particle composition is substantially non-immunogenic. Immunogenicity can be quantified, e.g., as described herein. In some embodiments, the non-cell particle composition comprises elevated levels of an immunosuppressive agent as compared to a reference particle, e.g., an otherwise similar particle but that does not contain exogenous CD24 or a biologically active portion. In some embodiments, the immunosuppressive agent is CD24. In some embodiments, the immunosuppressive agent is CD24 and CD47. In some embodiments, the elevated level is at least 5%, 10%, 20%, 30%, 40%, 50%, 60%, 70%, 80%, 90%, 2-fold, 3-fold, 5-fold, 10-fold, 20-fold, 50-fold, or 100-fold. In some embodiments, the non-cell particle composition comprises CD24 and CD47 that is absent from the reference particle.

In some embodiments, the non-cell particle composition has a reduction in immunogenicity as measured by a reduction in humoral response following one or more implantation of the non-cell particle derived into an appropriate animal model, e.g., an animal model described herein, compared to a humoral response following one or more implantation of a reference cell, e.g., an unmodified cell otherwise similar to the source cell, into an appropriate animal model, e.g., an animal model described herein. In some embodiments, the reduction in humoral response is measured in a serum sample by an anti-cell antibody titer, e.g., by ELISA.

In some embodiments, the serum sample from animals administered the non-cell particle composition has a reduction of 1%, 5%, 10%, 20%, 30%, 40%, 50%, 60%, 70%, 80%, 90%, or more of an anti-cell antibody titer compared to the serum sample from animals administered an unmodified cell. In some embodiments, the serum sample from animals administered the non-cell particle composition has an increased anti-cell antibody titer, e.g., increased by 1%, 2%, 5%, 10%, 20%, 30%, or 40% from baseline, e.g., wherein baseline refers to serum sample from the same animals before administration of the non-cell particle composition.

In some embodiments, immunogenicity of a non-cell particle composition is assayed by a serum inactivation assay (e.g., an assay that detects antibody-mediated neutralization or complement mediated degradation). In some embodiments, non-cell particle are not inactivated by serum or are inactivated at a level below a predetermined value. In some embodiments, serum of a non-cell particle-naive subject (e.g., human or mouse) is contacted with a test non-cell particle composition. In some embodiments, the serum of a subject that has received one or more administrations of non-cell particle, e.g., has received at least two administrations of non-cell particle, is contacted with the test non-cell particle composition. In embodiments, serum-exposed non-cell particles are then tested for ability to deliver a cargo to target cells. In some embodiments, the percent of cells that detectably comprise the cargo after treatment with serum-incubated non-cell particles is at least 50%, 60%, 70%, 80%, 90%, or 95% the percent of cells that detectably comprise the cargo after treatment with positive control non-cell particles not contacted with serum.

In some embodiments, immunogenicity of a non-cell particle composition is assayed by detecting complement activation in response to the non-cell particles. In some embodiments, the non-cell particles do not activate complement, or activate complement at a level below a predetermined value. In some embodiments, serum of a non-cell particle-naive subject (e.g., human or mouse) is contacted with a test non-cell particle composition. In some embodiments, the serum of a subject that has received one or more administrations of non-cell particles, e.g., has received at least two administrations of non-cell particles, is contacted with the test non-cell particle composition. In embodiments, the composition comprising serum and non-cell particles is then tested for an activated complement factor (e.g., C3a), e.g., by ELISA. In some embodiments, a non-cell particle described herein (e.g., elevated levels of a complement regulatory protein compared to a reference cell) undergoes reduced complement activation compared to an otherwise similar non-cell particle that does not contain exogenous CD24 or a biologically active portion, e.g., reduced by at least 5%, 10%, 20%, 30%, 40%, 50%, 60%, 70%, 80%, 90%, 95%, 98%, or 99%.

In some embodiments, the non-cell particles expressing CD24 or a biologically active portion thereof have a longer half-life in vivo compared to otherwise identical non-cell particles that do not express CD24 or a biologically active portion thereof. In some embodiments, the non-cell particles expressing CD24 and CD47 of a biologically active portion thereof have a longer half-life in vivo compared to otherwise identical non-cell particles that do not express CD24 and CD47 or a biologically active portion thereof.

In some embodiments, non-cell particles described herein evade phagocytosis and have a longer half-life when administered to a mammal. In some embodiments, the mammal is a human.

In some embodiments, the non-cell particle evades immune evasion through CD24 interaction with the inhibitory receptor sialic-acid-binding Ig-like lectin 10 (Siglec-10), which is expressed by tumor-associated macrophages. In some embodiments, the non-cell particle comprises immune evasion through CD24 and CD47 interaction with the inhibitory receptor sialic-acid-binding Ig-like lectin 10 (Siglec-10) and SIRPα, respectively.

In some embodiments, the non-cell particle composition has a reduction in macrophage phagocytosis, e.g., a reduction of 1%, 5%, 10%, 20%, 30%, 40%, 50%, 60%, 70%, 80%, 90%, or more in macrophage phagocytosis compared to a reference particle, e.g., an otherwise similar particle but not containing exogenous CD24 or a biologically active portion, wherein the reduction in macrophage phagocytosis is determined by assaying the phagocytosis index in vitro.

In some embodiments, non-cell particles delivered into the peripheral circulation evade capture and retention by the reticulo-endothelial system (RES) in order to reach target sites with high efficiency. The RES comprises a system of cells, primarily macrophages, which reside in solid organs such as the spleen, lymph nodes and the liver. In some embodiments, the non-cell particle is not captured by the scavenger system in circulation or by Kupffer cells in the sinus of the liver.

### VII. PHARMACEUTICAL COMPOSITIONS AND METHODS OF MANUFACTURE

The present disclosure also provides, in some aspects, a pharmaceutical composition comprising the non-cell particle composition described herein and pharmaceutically acceptable carrier. The pharmaceutical compositions can include any of the described non-cell particles.

In some embodiments, the non-cell particle meets a pharmaceutical or good manufacturing practices (GMP) standard. In some embodiments, the non-cell particle was made according to good manufacturing practices (GMP). In some embodiments, the non-cell particle has a pathogen level below a predetermined reference value, e.g., is substantially free of pathogens. In some embodiments, the non-cell particle has a contaminant level below a predetermined reference value, e.g., is substantially free of contaminants. In some embodiments, the non-cell particle has low immunogenicity.

In some embodiments, provided herein are the use of pharmaceutical compositions of the invention or salts thereof to practice the methods of the invention. Such a pharmaceutical composition may consist of at least one compound or conjugate of the invention or a salt thereof in a form suitable for administration to a subject, or the pharmaceutical composition may comprise at least one compound or conjugate of the invention or a salt thereof, and one or more pharmaceutically acceptable carriers, one or more additional ingredients, or some combination of these. In some embodiments, the compound or conjugate of the invention may be present in the pharmaceutical composition in the form of a physiologically acceptable salt, such as in combination with a physiologically acceptable cation or anion, as is well known in the art.

In some embodiments, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

In some embodiments, the relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. In some embodiments, the composition may comprise between 0.1% and 100% (w/w) active ingredient.

In some embodiments, pharmaceutical compositions that are useful in the methods of the invention may be suitably developed for oral, rectal, vaginal, parenteral, topical, pulmonary, intranasal, buccal, ophthalmic, or another route of administration. In some embodiments, a composition useful within the methods of the invention may be directly administered to the skin, vagina or any other tissue of a mammal. In some embodiments, formulations include liposomal preparations, resealed erythrocytes containing the active ingredient, and immunologically based formulations. In some embodiments, the route(s) of administration will be readily apparent to the skilled artisan and will depend upon any number of factors including the type and severity of the disease being treated, the type and age of the veterinary or human subject being treated, and the like.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, although the descriptions of pharmaceutical compositions provided herein are principally directed to pharmaceutical compositions that are suitable for ethical administration to humans, it will be understood by the skilled artisan that such compositions are generally suitable for administration to animals of all sorts. In some embodiments, modification of pharmaceutical compositions suitable for administration to humans in order to render the compositions suitable for administration to various animals is well understood, and the ordinarily skilled veterinary pharmacologist may design and perform such modification with merely ordinary, if any, experimentation. In some embodiments, subjects to which administration of the pharmaceutical compositions of the invention is contemplated include humans and other primates, mammals including commercially relevant mammals such as cattle, pigs, horses, sheep, cats, and dogs.

In some of any embodiments, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. In some embodiments, pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

In some embodiments, the carrier may be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity may be maintained, for example, by the use of a coating such as lecithin, by the maintenance of the required particle size in the case of dispersion and by the use of surfactants. In some embodiments, prevention of the action of microorganisms may be achieved by various antibacterial and antifungal agents, for example, parabens, chlorobutanol, phenol, ascorbic acid, thimerosal, and the like. In some embodiments, it is preferable to include isotonic agents, for example, sugars, sodium chloride, or polyalcohols such as mannitol and sorbitol, in the composition. In some embodiments, prolonged absorption of the injectable compositions may be brought about by including in the composition an agent that delays absorption, for example, aluminum monostearate or gelatin. In one embodiment, the pharmaceutically acceptable carrier is not DMSO alone.

In some embodiments, formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. In some embodiments, the pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. In some embodiments, pharmaceutical preparations may also be combined where desired with other active agents, e.g., other analgesic agents.

In some embodiments, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. In some embodiments, “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

In some embodiments, the composition of the invention may comprise a preservative from about 0.005% to 2.0% by total weight of the composition. In some embodiments, the preservative is used to prevent spoilage in the case of exposure to contaminants in the environment. In some embodiments, examples of preservatives useful in accordance with the invention included but are not limited to those selected from the group consisting of benzyl alcohol, sorbic acid, parabens, imidurea and combinations thereof. In some embodiments, a particularly preferred preservative is a combination of about 0.5% to 2.0% benzyl alcohol and 0.05% to 0.5% sorbic acid.

In some embodiments, the composition preferably includes an anti-oxidant and a chelating agent that inhibits the degradation of the compound. In some embodiments, antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. In some embodiments, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. In some embodiments, the chelating agent is useful for chelating metal ions in the composition that may be detrimental to the shelf life of the formulation. In some embodiments, other suitable and equivalent antioxidants and chelating agents may be substituted therefore as would be known to those skilled in the art.

In some embodiments, liquid suspensions may be prepared using conventional methods to achieve suspension of the active ingredient in an aqueous or oily vehicle. In some embodiments, aqueous vehicles include, for example, water, and isotonic saline. In some embodiments, oily vehicles include, for example, almond oil, oily esters, ethyl alcohol, vegetable oils such as arachis, olive, sesame, or coconut oil, fractionated vegetable oils, and mineral oils such as liquid paraffin. In some embodiments, liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. In some embodiments, oily suspensions may further comprise a thickening agent. In some embodiments, suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. In some embodiments, dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively).

Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

In some embodiments, liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. In some embodiments, liquid solutions of the pharmaceutical composition of the invention may comprise each of the components described with regard to liquid suspensions, it being understood that suspending agents will not necessarily aid dissolution of the active ingredient in the solvent. In some embodiments, aqueous solvents include, for example, water, and isotonic saline. In some embodiments, oily solvents include, for example, almond oil, oily esters, ethyl alcohol, vegetable oils such as arachis, olive, sesame, or coconut oil, fractionated vegetable oils, and mineral oils such as liquid paraffin.

In some embodiments, powdered and granular formulations of a pharmaceutical preparation of the invention may be prepared using known methods. In some embodiments, formulations may be administered directly to a subject, used, for example, to form tablets, to fill capsules, or to prepare an aqueous or oily suspension or solution by addition of an aqueous or oily vehicle thereto. In some of any embodiments, formulations may further comprise one or more of dispersing or wetting agent, a suspending agent, and a preservative. Additional excipients, such as fillers and sweetening, flavoring, or coloring agents, may also be included in these formulations.

In some embodiments, a pharmaceutical composition of the invention may also be prepared, packaged, or sold in the form of oil-in-water emulsion or a water-in-oil emulsion. In some embodiments, the oily phase may be a vegetable oil such as olive or arachis oil, a mineral oil such as liquid paraffin, or a combination of these. In some embodiments, compositions further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. In some embodiments, emulsions may also contain additional ingredients including, for example, sweetening or flavoring agents.

### VIII. METHODS OF DELIVERY AND TREATMENT

In some embodiments, the CD24-associated non-cell particles provided herein, e.g. retroviral particles, other non-cell particles, or fusosomes thereof, is capable of delivering (e.g., delivers) an exogenous agent to a target cell. The exogenous agent can be a protein, nucleic acid (e.g., mRNA), or small molecule. Exemplary exogenous agents that can be contained in a non-cell particle herein for delivery are described in Section V. Among provided methods herein are methods that comprise delivering an agent to a target cell. In some embodiments, the exogenous agent is an agent that is entirely heterologous or not produced or normally expressed by the target cell. In some embodiments, delivery of the exogenous agent to the target cell can provide a therapeutic effect to treat a disease or condition in the subject. The therapeutic effect may be by targeting, modulating or altering an antigen or protein present or expressed by the target cell that is associated with or involved in a disease or condition. The therapeutic effect may be by providing an exogenous agent in which the exogenous agent is a protein (or a nucleic acid encoding the protein, e.g., an mRNA encoding the protein) which is absent, mutant, or at a lower level than wild-type in the target cell. In some embodiments, the target cell is from a subject having a genetic disease, e.g., a monogenic disease, e.g., a monogenic intracellular protein disease.

The non-cell particles, e.g., fusosomes, retroviral vectors, VLPs, or pharmaceutical compositions, described herein can be administered to a subject, e.g., a mammal, e.g., a human.

In such embodiments, the subject may be at risk of, may have a symptom of, or may be diagnosed with or identified as having, a particular disease or condition (e.g., a disease or condition described herein). In some embodiments, the disease or condition may be one that is treated by delivery of the exogenous agent contained in the administered non-cell particle to a target cell in the subject.

This disclosure also provides, in certain aspects, a method of administering a non-cell particle composition to a subject (e.g., a human subject), a target tissue, or a cell, comprising administering to the subject, or contacting the target tissue or the cell with a non-cell particle composition comprising a plurality of non-cell particles described herein, a non-cell particle composition described herein, or a pharmaceutical composition described herein, thereby administering the non-cell particle composition to the subject

This disclosure also provides, in certain aspects, a method of delivering an exogenous agent, for instance a therapeutic agent (e.g., a polypeptide, a nucleic acid, a metabolite, an organelle, or a subcellular structure), to a subject, a target tissue, or a cell, comprising administering to the subject, or contacting the target tissue or the cell with, a plurality of non-cell particles described herein, a non-cell particle composition comprising a plurality of non-cell particles described herein, a non-cell particle composition described herein, or a pharmaceutical composition described herein, wherein the non-cell particle composition is administered in an amount and/or time such that the therapeutic agent is delivered. Exemplary exogenous agents that can be contained in a non-cell particle herein for delivery to a subject are described in Section V.

This disclosure also provides, in certain aspects, a method of delivering a function to a subject, a target tissue, or a cell, comprising administering to the subject, or contacting the target tissue or the cell with, a plurality of non-cell particles described herein, a non-cell particle composition comprising a plurality of non-cell particles described herein, a non-cell particle composition described herein, or a pharmaceutical composition described herein, wherein the non-cell particle composition is administered in an amount and/or time such that the function is delivered via delivery by the non-cell particle composition of an exogenous agent (e.g., therapeutic agent) to the target tissue or the cell.

Target cells from mammalian (e.g., human) tissue include cells from epithelial, connective, muscular, or nervous tissue or cells, and combinations thereof. Target mammalian (e.g., human) cells and organ systems include the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves)'; reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof. In some embodiments, a non-target cells or organ system is chosen from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves)'; reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof.

In some embodiments, the target cell or tissue is any such listed in any of WO 2020/102499, WO 2020/102485, WO 2019/222403, WO 2020/014209, and WO 2020/102503, each of which is hereby incorporated by reference in its entirety. In some embodiments, the target cell is a T cell. In some embodiments, the target cell is any of a CD4+T cell, a CD8+ T cell, an alpha beta T cell, a gamma delta T cell, a naive T cell, an effector T cell, a cytotoxic T cell (e.g., a CD8+ cytotoxic T cell), a regulatory T cell (e.g., a thymus-derived regulatory T cell, a peripherally derived regulatory T cell, a CD4+Foxp3+ regulatory T cell, or a CD4+FoxP3-type 1 regulatory T (Tr1) cell), a helper T cell (e.g., a CD4+ helper T cell, a Th1 cell, a Th2 cell, a Th3 cell, a Th9 cell, a Th17 cell, a Th22 cell, or a T follicular helper (Tfh) cell), a memory T cell (e.g., a stem cell memory T cell, a central memory T cell, or an effector memory T cell), a NKT cell, and a Mucosal associated invariant T (MAIT) cell.

A. Delivery

In some embodiments, the non-cell particle delivers the exogenous agent to at least 40%, 45%, 50%, 55%, 60%, 65%, 70%, 75%, 80%, 85%, 90%, 95%, 96%, 97%, 98%, or 99% of the number of cells in the target cell population. In some embodiments, the non-cell particle delivers at least 40%, 45%, 50%, 55%, 60%, 65%, 70%, 75%, 80%, 85%, 90%, 95%, 96%, 97%, 98%, or 99% of the exogenous agent to the target cell population.

In some embodiments, the non-cell particle delivers at least 40%, 45%, 50%, 55%, 60%, 65%, 70%, 75%, 80%, 85%, 90%, 95%, 96%, 97%, 98%, or 99% more of the exogenous agent to the target cell population compared to a non-target cell population. In some embodiments, the non-cell particle delivers more exogenous agent to the target cell population based on the non-cell particle comprising a fusogen or re-target fusogen that facilitates binding to the target cell population, but not the non-target cell population. The non-cell particle can comprise any of the exemplary fusogens and re-targeted fusogens described in Section IV herein. In some embodiments, when the plurality of non-cell particles are contacted with a cell population comprising target cells and non-target cells, the exogenous agent is present in at least 10-fold more target cells than non-target cells. In some embodiments, when the plurality of non-cell particles are contacted with a cell population comprising target cells and non-target cells, the exogenous agent is present at least 2-fold, 5-fold, 10-fold, 20-fold, or 50-fold higher in target cells than non-target cells and/or the exogenous agent is present at least 2-fold, 5-fold, 10-fold, 20-fold, or 50-fold higher in target cells than non-target cells. In some embodiments, the non-cell particles of the plurality fuse at a higher rate with a target cell than with a non-target cell by at least 50%.

In some embodiments, the non-cell particle, when contacted with a target cell population, delivers the exogenous agent to a target cell location other than an endosome or lysosome. In embodiments, less 50%, 40%, 30%, 20%, or 10% of the exogenous agent is delivered to an endosome or lysosome. In some embodiments, less than 10% of exogenous agent enters the cell by endocytosis. In some embodiments, the non-cell particle is capable of delivering (e.g., delivers) an exogenous agent, e.g., a protein, to the cytosol of a target cell.

In some embodiments, the non-cell particle is capable of delivering (e.g., delivers) an exogenous agent, e.g., a protein, to the cell membrane of a target cell. Similarly, in some embodiments, a method herein comprises delivering an exogenous agent to the cell membrane of a target cell. In some embodiments, delivering the protein comprises delivering a nucleic acid (e.g., mRNA) encoding the protein to the target cell such that the target cell produces the protein and localizes it to the membrane. In some embodiments, the non-cell particle comprises, or the method further comprises delivering, the protein, and fusion of the non-cell particle with the target cell transfers the protein to the cell membrane of the target cell. In some embodiments, the exogenous agent comprises a cell surface ligand or an antibody that binds a cell surface receptor.

In some embodiments, the non-cell particle further comprises, or the method further comprises delivering, a second exogenous agent that comprises or encodes a second cell surface ligand or antibody that binds a cell surface receptor, and optionally further comprising or encoding one or more additional cell surface ligands or antibodies that bind a cell surface receptor (e.g., 1, 2, 3, 4, 5, 10, 20, 50, or more). In some embodiments, the first exogenous agent and the second exogenous agent form a complex, wherein optionally the complex further comprises one or more additional cell surface ligands. In some embodiments, the exogenous agent comprises or encodes a cell surface receptor, e.g., an exogenous cell surface receptor. In some embodiments, the non-cell particle further comprises, or the method further comprises delivering, a second exogenous agent that comprises or encodes a second cell surface receptor, and optionally further comprises or encodes one or more additional cell surface receptors (e.g., 1, 2, 3, 4, 5, 10, 20, 50, or more cell surface receptors).

In some embodiments, the non-cell particle is capable of delivering (e.g., delivers) one or more cell surface receptors to a target cell (e.g., an immune cell). Similarly, in some embodiments, a method herein comprises delivering one or more cell surface receptors to a target cell. In some embodiments, the first exogenous agent and the second exogenous agent form a complex, wherein optionally the complex further comprises one or more additional cell surface receptors. In some embodiments, the exogenous agent comprises or encodes an antigen or an antigen presenting protein.

In some embodiments, the non-cell particle is capable of causing (e.g., causes) a target cell to secrete a protein, e.g., a therapeutic protein. In some embodiments, the non-cell particle is capable of delivering (e.g., delivers) a secreted exogenous agent, e.g., a secreted protein to a target site (e.g., an extracellular region), e.g., by delivering a nucleic acid (e.g., mRNA) encoding the protein to the target cell under conditions that allow the target cell to produce and secrete the protein. Similarly, in some embodiments, a method herein comprises delivering a secreted exogenous agent as described herein. In embodiments, the secreted protein comprises a protein therapeutic, e.g., an antibody molecule, a cytokine, or an enzyme. In embodiments, the secreted protein comprises an autocrine signalling molecule or a paracrine signalling molecule. In embodiments, the secreted exogenous agent comprises a secretory granule.

In some embodiments, the non-cell particle is capable of secreting (e.g., secretes) an exogenous agent, e.g., a protein. In some embodiments, the exogenous agent, e.g., secreted agent, is delivered to a target site in a subject. In some embodiments, the exogenous agent is a protein that cannot be made recombinantly or is difficult to make recombinantly. In some embodiments, the non-cell particle that secretes a protein is from a source cell selected from an MSC or a chondrocyte.

In some embodiments, the non-cell particle is capable of reprogramming (e.g., reprograms) a target cell (e.g., an immune cell), e.g., by delivering an exogenous agent selected from a transcription factor, a nucleic acid encoding a transcription factor, mRNA, or a plurality of said exogenous agents. Similarly, in some embodiments, a method herein comprises reprogramming a target cell. In embodiments, reprogramming comprises inducing a pancreatic endocrine cell to take on one or more characteristics of a pancreatic beta cell, by inducing a non-dopaminergic neuron to take on one or more characteristics of a dopaminergic neuron, or by inducing an exhausted T cell to take on one or more characteristics of a nonexhausted T cell, e.g., a killer T cell. In some embodiments, the exogenous agent comprises an antigen. In some embodiments, the non-cell particle comprises a first exogenous agent comprising an antigen and a second exogenous agent comprising an antigen presenting protein.

In some embodiments, a non-cell particle is capable of modifying, e.g., modifies, a target tumor cell, for instance by delivering an exogenous agent (protein or nucleic acid) or a nucleic encoding an exogenous agent. Similarly, in some embodiments, a method herein comprises modifying a target tumor cell. In embodiments, the non-cell particle delivers an mRNA encoding an immunostimulatory ligand, an antigen presenting protein, a tumor suppressor protein, or a pro-apoptotic protein. In some embodiments, the non-cell particle delivers an miRNA capable of reducing levels in a target cell of an immunosuppressive ligand, a mitogenic signal, or a growth factor.

In some embodiments, a non-cell particle delivers an exogenous agent that is immunomodulatory, e.g., immuno stimulatory.

In some embodiments, a non-cell particle is capable of causing (e.g., causes) the target cell to present an antigen, for instance by delivering an exogenous agent comprising an antigen or a nucleic acid encoding the antigen. Similarly, in some embodiments, a method herein comprises presenting an antigen on a target cell. In some embodiments, the non-cell particle promotes regeneration in a target tissue. Similarly, in some embodiments, a method herein comprises promoting regeneration in a target tissue. In embodiments, the target cell is a cardiac cell, e.g., a cardiomyocyte (e.g., a quiescent cardiomyocyte), a hepatoblast (e.g., a bile duct hepatoblast), an epithelial cell, a naive T cell, a macrophage (e.g., a tumor infiltrating macrophage), or a fibroblast (e.g., a cardiac fibroblast). In embodiments, the source cell is a T cell (e.g., a Treg), a macrophage, or a cardiac myocyte.

In some embodiments, the non-cell particle is capable of delivering (e.g., delivers) a nucleic acid to a target cell, e.g., to stably modify the genome of the target cell, e.g., for gene therapy. Similarly, in some embodiments, a method herein comprises delivering a nucleic acid to a target cell. In some embodiments, the target cell has an enzyme deficiency, e.g., comprises a mutation in an enzyme leading to reduced activity (e.g., no activity) of the enzyme.

In some embodiments, the non-cell particle is capable of delivering (e.g., delivers) a reagent that mediates a sequence specific modification to DNA (e.g., Cas9, ZFN, or TALEN) in the target cell. Similarly, in some embodiments, a method herein comprises delivering the reagent to the target cell. In embodiments, the target cell is a CNS cell.

In some embodiments, the non-cell particle is capable of delivering (e.g., delivers) a nucleic acid to a target cell, e.g., to transiently modify gene expression in the target cell.

In some embodiments, the non-cell particle is capable of delivering (e.g., delivers) a protein to a target cell, e.g., to transiently rescue a protein deficiency. Similarly, in some embodiments, a method herein comprises delivering a protein to a target cell. In embodiments, the protein is a membrane protein (e.g., a membrane transporter protein), a cytoplasmic protein (e.g., an enzyme), or a secreted protein (e.g., an immunosuppressive protein).

In some embodiments, the non-cell particle is capable of intracellular molecular delivery, e.g., delivers a protein exogenous agent to a target cell. Similarly, in some embodiments, a method herein comprises delivering a molecule to an intracellular region of a target cell. In embodiments, the protein exogenous agent is an inhibitor. In some embodiments, the protein exogenous agent comprises a nanobody, scFv, camelid antibody, peptide, macrocycle, or small molecule.

In some embodiments, the non-cell particle comprises on its membrane one or more cell surface ligands (e.g., 1, 2, 3, 4, 5, 10, 20, 50, or more cell surface ligands), said cell surface ligands to be presented by the non-cell particle to a target cell. Similarly, in some embodiments, a method herein comprises presenting one or more cell surface ligands to a target cell. In some embodiments, the non-cell particle having a cell surface ligand is from a source cell chosen from a neutrophil (e.g., and the target cell is a tumor-infiltrating lymphocyte), dendritic cell (e.g., and the target cell is a naive T cell), or neutrophil (e.g., and the target is a tumor cell or virus-infected cell). In some embodiments the non-cell particle comprises a membrane complex, e.g., a complex comprising at least 2, 3, 4, or 5 proteins, e.g., a homodimer, heterodimer, homotrimer, heterotrimer, homotetramer, or heterotetramer. In some embodiments, the non-cell particle comprises an antibody, e.g., a toxic antibody, e.g., the non-cell particle is capable of delivering the antibody to the target site, e.g., by homing to a target site. In some embodiments, the source cell is an NK cell or neutrophil.

In some embodiments, a method herein comprises causing ligand presentation on the surface of a target cell by presenting cell surface ligands on the non-cell particle. In some embodiments, the non-cell particle is capable of causing cell death of the target cell. In some embodiments, the non-cell particle is from a NK source cell.

In some embodiments, a non-cell particle or target cell is capable of phagocytosis (e.g., of a pathogen). Similarly, in some embodiments, a method herein comprises causing phagocytosis.

In some embodiments, a non-cell particle senses and responds to its local environment. In some embodiments, the non-cell particle is capable of sensing level of a metabolite, interleukin, or antigen.

In embodiments, a non-cell particle is capable of chemotaxis, extravasation, or one or more metabolic activities. In embodiments, the metabolic activity is selected from kyneurinine, gluconeogenesis, prostaglandin fatty acid oxidation, adenosine metabolism, urea cycle, and thermogenic respiration. In some embodiments, the source cell is a neutrophil and the non-cell particle is capable of homing to a site of injury. In some embodiments, the source cell is a macrophage and the non-cell particle is capable of phagocytosis. In some embodiments, the source cell is a brown adipose tissue cell and the non-cell particle is capable of lipolysis.

In some embodiments, the non-cell particle comprises (e.g., is capable of delivering to the target cell) a plurality of exogenous agents (e.g., at least 2, 3, 4, 5, 10, 20, or 50 exogenous agents) or nucleic acids encoding a plurality of exogenous agents. In embodiments, the non-cell particle comprises an inhibitory nucleic acid (e.g., siRNA or miRNA) and an mRNA.

In some embodiments, the non-cell particle comprises (e.g., is capable of delivering to the target cell) a membrane protein or a nucleic acid encoding the membrane protein. In embodiments, the non-cell particle is capable of reprogramming or transdifferentiating a target cell, e.g., the non-cell particle comprises one or more agents that induce reprogramming or transdifferentiation of a target cell.

In some embodiments, the non-cell particle, e.g., fusosome, fuses at a higher rate with a target cell than with a non-target cell based on the non-cell particle comprising a fusogen or re-target fusogen that facilitates binding to the target cell, but not the non-target cell. The non-cell particle can comprise any of the exemplary fusogens and re-targeted fusogens described in Section IV. In some embodiments, the non-cell particle, e.g., fusosome, fuses at a higher rate with a target cell than with a non-target cell, e.g., by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold. In some embodiments, the non-cell particle, e.g., fusosome, fuses at a higher rate with a target cell than with other non-cell particles, e.g., by at least 10%, 20%, 30%, 40%, 50%, 60%, 70%, 80%, or 90%. In some embodiments, the non-cell particle, e.g., fusosome, fuses with target cells at a rate such that an exogenous agent or nucleic acid encoding an exogenous agent in the non-cell particle is delivered to at least 10%, 20%, 30%, 40%, 50%, 60%, 70%, 80%, or 90%, of target cells after 24, 48, or 72 hours. In embodiments, the amount of targeted fusion is about 30%-70%, 35%-65%, 40%-60%, 45%-55%, or 45%-50%. In embodiments, the amount of targeted fusion is about 20%-40%, 25%-35%, or 30%-35%.

In some embodiments, the fusogen is present at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, at least 10%, 20%, 30%, 40%, 50%, 60%, 70%, 80%, 90%, 95%, 96%, 97%, 98%, or 99% of the fusogen comprised by the non-cell particle is disposed in the cell membrane. In embodiments, the non-cell particle also comprises fusogen internally, e.g., in the cytoplasm or an organelle. In some embodiments, the fusogen comprises (or is identified as comprising) about 0.1%, 0.2%, 0.3%, 0.4%, 0.5%, 0.6%, 0.7%, 0.8%, 0.9%, 1%, 5%, 10%, 11%, 12%, 13%, 14%, 15%, 20%, or more, or about 1-30%, 5-20%, 10-15%, 12-15%, 13-14%, or 13.6% of the total protein in a non-cell particle, e.g., as determined by a mass spectrometry assay. In embodiments, the fusogen comprises (or is identified as comprising) about 13.6% of the total protein in the non-cell particle. In some embodiments, the fusogen is (or is identified as being) more or less abundant than one or more additional proteins of interest. In an embodiment, the fusogen has (or is identified as having) a ratio to EGFP of about 140, 145, 150, 151, 152, 153, 154, 155, 156, 157 (e.g., 156.9), 158, 159, 160, 165, or 170. In another embodiment, the fusogen has (or is identified as having) a ratio to CD63 of about 2700, 2800, 2900, 2910 (e.g., 2912), 2920, 2930, 2940, 2950, 2960, 2970, 2980, 2990, or 3000, or about 1000-5000, 2000-4000, 2500-3500, 2900-2930, 2910-2915, or 2912.0, e.g., by a mass spectrometry assay. In an embodiment, the fusogen has (or is identified as having) a ratio to ARRDC1 of about 600, 610, 620, 630, 640, 650, 660 (e.g., 664.9), 670, 680, 690, or 700. In another embodiment, the fusogen has (or is identified as having) a ratio to GAPDH of about 50, 55, 60, 65, 70 (e.g., 69), 75, 80, or 85, or about 1-30%, 5-20%, 10-15%, 12-15%, 13-14%, or 13.6%. In another embodiment, the fusogen has (or is identified as having) a ratio to CNX of about 500, 510, 520, 530, 540, 550, 560 (e.g., 558.4), 570, 580, 590, or 600, or about 300-800, 400-700, 500-600, 520-590, 530-580, 540-570, 550-560, or 558.4, e.g., by a mass spectrometry assay.

In some embodiments, the non-cell particle comprises a therapeutic agent at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the non-cell particle comprises a protein therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the non-cell particle comprises a nucleic acid therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the non-cell particle comprises a DNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the non-cell particle comprises an RNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the non-cell particle comprises a nucleic acid encoding an exogenous protein, e.g. a therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the non-cell particle comprises an exogenous therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the non-cell particle comprises an exogenous protein therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the non-cell particle comprises an exogenous nucleic acid (e.g., DNA or RNA) therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the ratio of the copy number of the fusogen to the copy number of the therapeutic agent is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000

In some embodiments, the non-cell particle delivers to a target cell at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a exogenous agent. In some embodiments, the non-cell particle delivers to a target cell at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a therapeutic agent. In some embodiments, the non-cell particle delivers to a target cell at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a protein therapeutic agent. In some embodiments, the non-cell particle delivers to a target cell at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a nucleic acid therapeutic agent. In some embodiments, the non-cell particle delivers to a target cell at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of an RNA therapeutic agent. In some embodiments, the non-cell particle delivers to a target cell at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a DNA therapeutic agent.

In some embodiments, the non-cell particle delivers to a target cell at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the non-cell particle. In some embodiments, the non-cell particles, e.g., fusosomes, that fuse with the target cell(s) deliver to the target cell an average of at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the non-cell particles that fuse with the target cell(s). In some embodiments, the non-cell particle composition delivers to a target tissue at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the non-cell particle composition.

In some embodiments, the non-cell particle comprises 0.00000001 mg fusogen to 1 mg fusogen per mg of total protein in non-cell particle, e.g., 0.00000001-0.0000001, 0.0000001-0.000001, 0.000001-0.00001, 0.00001-0.0001, 0.0001-0.001, 0.001-0.01, 0.01-0.1, or 0.1-1 mg fusogen per mg of total protein in non-cell particle. In some embodiments, the non-cell particle comprises 0.00000001 mg fusogen to 5 mg fusogen per mg of lipid in non-cell particle, e.g., 0.00000001-0.0000001, 0.0000001-0.000001, 0.000001-0.00001, 0.00001-0.0001, 0.0001-0.001, 0.001-0.01, 0.01-0.1, 0.1-1, or 1-5 mg fusogen per mg of lipid in non-cell particle.

B. Treatment and Uses

In some embodiments, the CD24-associated non-cell particles provided herein, e.g. non-cell particle, e.g. retroviral particles other non-cell particles or fusosomes thereof, or pharmaceutical compositions thereof as described herein can be administered to a subject, e.g. a mammal, e.g. a human. In some embodiments, the administration delivers the non-cell particles to a target cell in the subject. In such embodiments, the subject may be at risk of, may have a symptom of, or may be diagnosed with or identified as having, a particular disease or condition. In some embodiments, the methods thereby treat the disease or condition or disorder in the subject. In one embodiment, the subject has cancer. In one embodiment, the subject has an infectious disease. In some embodiments, the non-cell particle, e.g. retroviral particles other non-cell particles or fusosomes thereof, contains nucleic acid sequences encoding an exogenous agent for treating the disease or condition in the subject. For example, the exogenous agent is one that targets or is specific for a protein of a neoplastic cells and the non-cell particle, e.g. retroviral particles other non-cell particles or fusosomes thereof, is administered to a subject for treating a tumor or cancer in the subject. In another example, the exogenous agent is an inflammatory mediator or immune molecule, such as a cytokine, and the non-cell particle, e.g. retroviral particles other non-cell particles or fusosomes thereof, is administered to a subject for treating any condition in which it is desired to modulate (e.g. increase) the immune response, such as a cancer or infectious disease. In some embodiments, the CD24-associated non-cell particle, e.g. retroviral particles other non-cell particles or fusosomes thereof, is administered in an effective amount or dose to effect treatment of the disease, condition or disorder.

Provided herein are uses of any of the provided CD24-associated non-cell particles, e.g. retroviral particles other non-cell particles or fusosomes thereof, in such methods and treatments, and in the preparation of a medicament in order to carry out such therapeutic methods. In some embodiments, the methods are carried out by administering the non-cell particle, e.g. retroviral particles other non-cell particles or fusosomes thereof, or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intracavity, and subcutaneous) administration. In some embodiments, the non-cell particles may be administered alone or formulated as a pharmaceutical composition. In some embodiments, the non-cell particles or pharmaceutical compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the non-cell particles may be administered in the form of a unit-dose composition, such as a unit dose oral, parenteral, transdermal or inhaled composition.

In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, the administration of the compositions of the present invention to a subject, preferably a mammal, more preferably a human, may be carried out using known procedures, at dosages and for periods of time effective to prevent or treat disease. In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. In some embodiments, the effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

In some embodiments, the compound may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

In some embodiments, dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian could start doses of the compounds of the invention employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. In some embodiments, dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. In some embodiments, the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the compositions provided herein containing a provided non-cell particle such as any of the viral vectors or virus-based particles described herein, can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25(2):115-25. 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10¹⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 01m infectious units, inclusive. In some embodiments, the dosage of administration of a viral vector or virus-like particle is from about 109 to about 10¹⁵ infectious units, inclusive In some embodiments, the dosage of administration of a viral vector or virus-like particle is from about 10W to about 109 infectious units. In some embodiments, the dosage of administration of a viral vector or virus-like particle is from about 106 to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, 1.0×10¹⁰ infectious units, 5.0×10¹⁰ infectious units, 1.×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID₅₀) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 1010 plaque forming units (pfu), inclusive. In some embodiments, the dosage of administration of a viral vector or virus-like particle is from about 10⁹ to about 10¹⁵ pfu, inclusive In some embodiments, the dosage of administration of a viral vector or virus-like particle is from about 10⁵ to about 10⁹ pfu. In some embodiments, the dosage of administration of a viral vector or virus-like particle is from about 10⁶ to about 109 pfu. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some aspects, the dosage of administration of a vehicle within the pharmaceutical compositions provided herein varies depending on a subject's body weight. For example, a composition may be formulated as GC/kg, infectious units/kg, pfu/kg, etc. In some aspects, the dosage at which a therapeutic effect is obtained is from at or about 108 GC/kg to at or about 10¹⁴ GC/kg of the subject's body weight, inclusive. In some aspects, the dosage at which a therapeutic effect is obtained is at or about 108 GC/kg of the subject's body weight (GC/kg).

In some of any embodiments, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors

In some embodiments, compounds for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound is from about 1 mg and about 2,500 mg. In some embodiments, a dose of a compound used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In some of any embodiments, the present disclosure is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition may be contained on the packaging containing the pharmaceutical composition, and as such the instructions form an increased functional relationship to the packaged product. In some embodiments, instructions may contain information pertaining to the compound's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some embodiments, delivery of a non-cell particle composition described herein may induce or block cellular differentiation, de-differentiation, or trans-differentiation. In some embodiments, the target mammalian cell may be a precursor cell. In some embodiments, the target mammalian cell may be a differentiated cell, and the cell fate alteration includes driving de-differentiation into a pluripotent precursor cell, or blocking such de-differentiation. In some embodiments, the effective amounts of a non-cell particle described herein encoding a cell fate inductive molecule or signal is introduced into a target cell under conditions such that an alteration in cell fate is induced. In some embodiments, a non-cell particle described herein is useful to reprogram a subpopulation of cells from a first phenotype to a second phenotype. In some embodiments, reprogramming may be temporary or permanent. In some embodiments, the reprogramming induces a target cell to adopt an intermediate phenotype.

Provided herein are methods of reducing cellular differentiation in a target cell population. In some embodiments, a target cell population containing one or more precursor cell types is contacted with a non-cell particles composition described herein, under conditions such that the composition reduces the differentiation of the precursor cell. In certain embodiments, the target cell population contains injured tissue in a mammalian subject or tissue affected by a surgical procedure. The precursor cell is, e.g., a stromal precursor cell, a neural precursor cell, or a mesenchymal precursor cell.

In some embodiments, the non-cell particle composition comprising a cargo, may be used to deliver such cargo to a cell tissue or subject. In some embodiments, delivery of a cargo by administration of a non-cell particle composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the non-cell particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the non-cell particle composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the non-cell particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered or contacted with a cell, e.g., a cell preparation. In some embodiments, the cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the non-cell particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of non-cell particles are from the same subject that is administered a non-cell particle composition. In other embodiments, they are different. In some embodiments, the source of non-cell particles and recipient tissue may be autologous (from the same subject) or heterologous (from different subjects). In some embodiments, the donor tissue for non-cell particle compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the non-cell particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the non-cell particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 DOI: 10.1038/srepO1462). In some embodiments, the non-cell particles is co-administered with an inhibitor of sypressyn, e.g., a siRNA or inhibitory antibody.

### IX. EXEMPLARY EMBODIMENTS

Among the provided embodiments are:

1. A non-cell particle comprising CD24 or a biologically active portion thereof on an exposed surface of the particle, wherein the non-cell particle is 1 μm or smaller.

2. The non-cell particle of embodiment 1, wherein the CD24 or the biologically active portion thereof binds Siglec-10.

3. The non-cell particle of embodiment 1 or embodiment 2, wherein the CD24 or biologically active portion thereof is human.

4. The non-cell particle of any of embodiments 1-3, wherein the CD24 or biologically active portion thereof:

(i) comprises the sequence set forth in SEQ ID NO: 2;

(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds Siglec-10; or

(iii) a binding portion of (i) or (ii) that binds to Siglec-10.

5. The non-cell particle of any of embodiments 1-4, wherein the CD24 or biologically active portion thereof comprises the sequence set forth in SEQ ID NO:2.

6. The non-cell particle of any of embodiments 1-5, wherein the CD24 or biologically active portion is displayed on an exposed surface of the particle via a Glycosylphosphatidylinositol (GPI) membrane anchor.

7. The non-cell particle of any of embodiments 1-6, wherein the CD24 or the biologically active portion thereof is encoded by a nucleic molecule encoding the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds Siglec-10.

8. The non-cell particle of any of embodiments 1-6, wherein the CD24 or the biologically active portion thereof is encoded by a nucleic molecule encoding the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds Siglec-10.

9. The non-cell particle of any of embodiments 1-5, wherein the CD24 or biologically active portion is displayed on an exposed surface of the particle via a transmembrane domain.

10. The non-cell particle of any of embodiments 1-9, wherein the CD24 or biologically active portion is a glycoprotein

11. The non-cell particle of any of embodiments 1-10, wherein the CD24 or biologically active portion is sialylated.

12. The non-cell particle of any of embodiments 1-11, wherein the CD24 or biologically active portion comprises an α2-3-linked sialoside and/or an α2-6-linked sialoside.

13. The non-cell particle of any of embodiments 1-12, wherein the CD24 or biologically active portion has a molecular weight of between at or about 35 kDa and at or about 45 kDa.

14. The non-cell particle of any of embodiments 1-13, wherein the non-cell particle further comprises a CD47 or a biologically active portion thereof on an exposed surface of the non-cell particle.

15. The non-cell particle of embodiment 14, wherein the CD47 or biologically active portion binds to SIRPα.

16. The non-cell particle of embodiment 14 or embodiment 15, wherein the CD47 or biologically active portion is human.

17. The non-cell particle of any one of embodiments 14-16, wherein the CD47 or biologically active portion comprises the extracellular domain or CD47 or a binding portion thereof that binds to SIRPα.

18. The non-cell particle of any of embodiments 14-17, wherein the CD47 or biologically active portion thereof:

(i) comprises the sequence set forth in SEQ ID NO: 7;

(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or

(iii) a binding portion of (i) or (ii) that binds to SIRPα.

19. The non-cell particle of any of embodiments 14-18, wherein the CD47 or biologically active portion thereof comprises the sequence set forth in SEQ ID NO:7.

20. The non-cell particle of any of embodiments 14-18, wherein the CD47 or biologically active portion thereof comprises the sequence set forth in SEQ ID NO:11.

21. The non-cell particle of any of embodiments 14-18 and 20, wherein the CD47 or biologically active portion thereof comprises the sequence set forth in SEQ ID NO:9.

22. The non-cell particle of any of embodiments 14-18 and 20, wherein the CD47 or biologically active portion thereof comprises the sequence set forth in SEQ ID NO:10.

23. The non-cell particle of any of embodiments 14-18 and 20, wherein the CD47 or biologically active portion thereof comprises the sequence set forth in SEQ ID NO:12.

24. The non-cell particle of any of embodiments 14-23, wherein the CD47 or biologically active portion is displayed on an exposed surface of the particle via a transmembrane domain.

25. The non-cell particle of any of embodiments 14-19 and 24, wherein the CD47 or biologically active portion comprises SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα.

26. The non-cell particle of any of embodiments 14-19, 24 and 25, wherein the CD47 or the biologically active portion thereof is encoded by a nucleic molecule encoding the sequence set forth in SEQ ID NO:5 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5 that binds to SIRPα.

27. The non-cell particle of any of embodiments 1-26, wherein the non-cell particle is a synthetic particle, a viral particle or a cell-derived particle.

28. The non-cell particle of any of embodiments 1-17, wherein further comprising a nucleic acid comprising a payload gene encoding an exogenous agent.

29. The non-cell particle of any of embodiments 1-28, wherein the non-cell particle is a synthetic particle selected from the group consisting of a liposome, a microparticle, a nanoparticle, a nanogel, a dendrimer and a dendrisome.

30. The non-cell particle of any of embodiments 1-28, wherein the exposed surface is a lipid bilayer and the non-cell particle further comprises a lumen comprising a cytosol, wherein the lumen is surrounded by the lipid bilayer.

31. The non-cell particle of embodiment 30, wherein the lumen further comprises a nucleic acid comprising a payload gene encoding an exogenous agent.

32. The non-cell particle of embodiment 30 or embodiment 31, wherein the non-cell particle is a fusosome and the lipid bilayer further comprises a fusogen.

33. The non-cell particle of any of embodiments 1-28 and 30-32, wherein the non-cell particle is derived from a source cell.

34. The non-cell particle of any of embodiments 1-33, wherein the non-cell particle does not comprise a nucleus.

35. The non-cell particle of any of embodiments 1-28 and 30-32, wherein the non-cell particle is a virus particle or a virus-like particle (VLP).

36. The non-cell particle of embodiments 35, wherein the virus particle or virus-like particle is a retroviral particle or retrovirus-like particle.

37. The non-cell particle of embodiment 36, wherein the retroviral particle or retrovirus-like particle is a lentiviral particle or a lentiviral-like particle.

38. The non-cell particle of any of embodiments 35-37, comprising a fusogen that is a viral fusogen selected from a Class I viral membrane fusion protein, a Class II viral membrane protein, a Class II viral membrane fusion protein, a viral membrane glycoprotein, or a viral envelope protein.

39. The non-cell particle of embodiment 38, wherein the fusogen is endogenous to the virus.

40. The non-cell particle of embodiment 38, wherein the fusogen is a pseudotyped fusogen.

41. The non-cell particle of any of embodiments 32-40, wherein the fusogen is a re-targeted fusogen that binds to a target cell.

42. The non-cell particle of embodiment 41, wherein the fusogen comprises a targeting moiety that binds to the target cell.

43. The non-cell particle of any of embodiments 35-42, wherein the virus or virus-like particle further comprising a lumen comprising a nucleic acid.

44. The non-cell particle of embodiment 43, wherein the nucleic acid comprises a viral nucleic acid comprising one or more of (e.g., all of) the following nucleic acid sequences:

5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

45. The non-cell particle of any of embodiments 35-44, wherein the non-cell particle is a virus-like particle (e.g. retrovirus-like particle) that is a replication defective.

46. A pseudotyped lentivirus or lentiviral-like particle comprising CD24 or a biologically active portion thereof on an exposed surface of the lentiviral particle.

47. The pseudotyped lentivirus or lentiviral-like particle of embodiment 46, wherein the particle is pseudotyped with a vesicular stomatitis virus envelope glycoprotein (VSV-G).

48. The pseudotyped lentivirus or lentiviral particle of embodiment 46, wherein the lentiviral particle is pseudotyped with a protein derived from an envelope glycoprotein of a virus of the Paramyxovirus family.

49. The pseudotyped lentivirus or lentiviral-like particle of embodiment 46, wherein the particle is pseudotyped with a cell targeting fusion protein comprising a protein derived from a Paramyxoviridae envelope protein G or H or a biologically active portion thereof and at least one cell targeting domain.

50. The pseudotyped lentivirus or lentiviral particle of embodiment 48 or embodiment 49, wherein the virus of the Paramyxovirus family is a Henipavirus or is a Morbillivirus.

51. The pseudotyped lentivirus or lentiviral particle of any of embodiments 48-50, wherein the envelope glycoprotein is an envelope glycoprotein G or H or a biologically active portion thereof.

52. The pseudotyped lentivirus or lentiviral-like particle of any of embodiments 48-51, wherein the envelope glycoprotein is Nipah virus G (Niv-G) protein or a biologically active portion thereof.

53. The pseudotyped lentivirus or lentiviral-like particle of any of embodiments 48-51, wherein the envelope glycoprotein is a Hendra virus G protein or a biologically active portion thereof.

54. The pseudotyped lentivirus or lentiviral-like particle of any of embodiments 48-51, wherein the envelope glycoprotein is a measles virus glycoprotein or a biologically active portion thereof.

55. The pseudotyped lentivirus or lentiviral-like particle of any of embodiments 48-54, wherein said protein derived from an envelope glycoprotein G or H of a virus of the Paramyxoviridae family is at least partially unable to bind at least one natural receptor of said envelope glycoprotein G or H.

56. The pseudotyped lentivirus or lentiviral-like particle of any of embodiments 48-55, further comprising an F protein molecule or a biologically active portion thereof from a Paramyxovirus.

57. The pseudotyped lentivirus or lentiviral-like particle of embodiment 56, wherein the Paramyxovirus is a Henipavirus.

58. The pseudotyped lentivirus or lentiviral-like particle of embodiment 56 or embodiment 57, wherein the protein protein molecule or a biologically active portion is a NiV-F protein or a biologically active portion.

59. The pseudotyped lentivirus or lentiviral-like particle of any of embodiments 46-58, wherein the CD24 or biologically active portion thereof:

(i) comprises the sequence set forth in SEQ ID NO: 2;

(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds to Siglec-10; or

(iii) a binding portion of (i) or (ii) that binds to Siglec-10.

60. The pseudotyped lentivirus or lentiviral-like particle of any of embodiments 46-59, wherein the CD24 or biologically active portion thereof comprises the sequence set forth in SEQ ID NO:2.

61. The pseudotyped lentivirus or lentiviral-like particle of any of embodiments 46-60, wherein the CD24 or biologically active portion is displayed on an exposed surface of the particle via a Glycosylphosphatidylinositol (GPI) membrane anchor.

62. The pseudotyped lentivirus or lentiviral-like particle of any of embodiments 46-61, wherein the CD24 or the biologically active portion thereof is encoded by a nucleic molecule encoding the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds to Siglec-10.

63. The pseudotyped lentivirus or lentiviral-like particle of any of embodiments 46-62, wherein the CD24 or the biologically active portion thereof is encoded by a nucleic molecule encoding the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds to Siglec-10.

64. The pseudotyped lentivirus or lentiviral-like particle of any of embodiments 46-60, wherein the CD24 or biologically active portion is displayed on an exposed surface of the particle via a transmembrane domain.

65. The pseudotyped lentivirus or lentiviral-like particle of any of embodiments 46-64, wherein the CD24 or biologically active portion is a glycoprotein

66. The pseudotyped lentivirus or lentiviral-like particle of any of embodiments 46-65, wherein the CD24 or biologically active portion is sialylated.

67. The pseudotyped lentivirus or lentiviral-like particle of any of embodiments 46-66, wherein the CD24 or biologically active portion comprises an α2-3-linked sialoside and/or an α2-6-linked sialoside.

68. The pseudotyped lentivirus or lentiviral-like particle of any of embodiments 46-67, wherein the CD24 or biologically active portion has a molecular weight of between at or about 35 kDa and at or about 45 kDa.

69. The pseudotyped lentivirus or lentiviral-like particle of any of embodiments 46-68, wherein the non-cell particle further comprises a CD47 or a biologically active portion thereof on an exposed surface of the non-cell particle.

70. The pseudotyped lentivirus or lentiviral-like particle of embodiment 69, wherein the CD47 or biologically active portion binds to SIRPα.

71. The pseudotyped lentivirus or lentiviral-like particle of embodiment 69 or embodiment 70, wherein the CD47 or biologically active portion is human.

72. The pseudotyped lentivirus or lentiviral-like particle of any one of embodiments 69-71, wherein the CD47 or biologically active portion comprises the extracellular domain or CD47 or a binding portion thereof that binds to SIRPα.

73. The pseudotyped lentivirus or lentiviral-like particle of any of embodiments 69-72, wherein the CD47 or biologically active portion thereof:

(i) comprises the sequence set forth in SEQ ID NO: 7;

(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or

(iii) a binding portion of (i) or (ii) that binds to SIRPα.

74. The pseudotyped lentivirus or lentiviral-like particle of any of embodiments 69-73, wherein the CD47 or biologically active portion thereof comprises the sequence set forth in SEQ ID NO:7.

75. The pseudotyped lentivirus or lentiviral-like particle of any of embodiments 69-73, wherein the CD47 or biologically active portion thereof comprises the sequence set forth in SEQ ID NO:11.

76. The pseudotyped lentivirus or lentiviral-like particle of any of embodiments 69-73 and 75, wherein the CD47 or biologically active portion thereof comprises the sequence set forth in SEQ ID NO:9.

77. The pseudotyped lentivirus or lentiviral-like particle of any of embodiments 69-73 and 75, wherein the CD47 or biologically active portion thereof comprises the sequence set forth in SEQ ID NO:10.

78. The pseudotyped lentivirus or lentiviral-like particle of any of embodiments 69-73 and 77, wherein the CD47 or biologically active portion thereof comprises the sequence set forth in SEQ ID NO:12.

79. The pseudotyped lentivirus or lentiviral-like particle of any of embodiments 69-78, wherein the CD47 or biologically active portion is displayed on an exposed surface of the particle via a transmembrane domain.

80. The pseudotyped lentivirus or lentiviral-like particle of any of embodiments 69-74 and 79, wherein the CD47 or biologically active portion comprises SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα.

81. The pseudotyped lentivirus or lentiviral-like particle of any of embodiments 69-74, 79 and 80, wherein the CD47 or the biologically active portion thereof is encoded by a nucleic molecule encoding the sequence set forth in SEQ ID NO:5 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5 that binds to SIRPα.

82. The non-cell particle of any of embodiments 1-45 or the pseudotyped lentivirus or lentiviral-like particle of any of embodiments 46-81, further comprising a nucleic acid comprising a payload gene encoding an exogenous agent.

83. The non-cell particle of embodiment 28, embodiment 31 or embodiment 82 or the pseudotyped lentivirus or lentiviral-like particle of embodiment 82, wherein the exogenous agent encodes a therapeutic agent or a diagnostic agent.

84. The non-cell particle of any of embodiments 1-45, 82 and 83 or the pseudotyped lentivirus or lentiviral-like particle of any of embodiments 46-83, wherein phagocytosis of the particle by a phagocytic cells, optionally a macrophage, is reduced compared to a reference particle that is otherwise similar but does not comprise the CD24 or biologically active portion, optionally wherein phagocytosis is reduced by at least 10%, 20%, 30%, 40%, 50%, 60%, 70%, 80%, or 90%.

85. The non-cell particle of any of embodiments 1-45 and 82-84 or the pseudotyped lentivirus or lentiviral-like particle of any of embodiments 46-84, wherein the half-life of the particle in vivo is increased compared to a reference particle that is otherwise similar but does not comprise the CD24 or biologically active portion, optionally wherein the half-life is increased by at least 10%, 20%, 30%, 40%, 50%, 60%, 70%, 80%, or 90%.

86. A polynucleotide comprising a first nucleic acid sequence encoding CD24 or a biologically active portion and a second nucleic acid encoding CD47 or a biologically active portion.

87. The polynucleotide of embodiment 86, wherein the encoded CD24 or the biologically active portion thereof binds Siglec-10.

88. The polynucleotide of embodiment 86 or embodiment 87, wherein the encoded CD24 or biologically active portion thereof is human.

89. The polynucleotide of any of embodiments 84-88, wherein the encoded CD24 or biologically active portion thereof:

(i) comprises the sequence set forth in SEQ ID NO: 2;

(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds to Siglec-10; or

(iii) a binding portion of (i) or (ii) that binds to Siglec-10.

90. The polynucleotide of any of embodiments 84-89, wherein the encoded CD24 or biologically active portion thereof comprises the sequence set forth in SEQ ID NO:2.

91. The polynucleotide of any of embodiments 84-90, wherein the encoded CD24 or biologically active portion is displayed on an exposed surface of the particle via a Glycosylphosphatidylinositol (GPI) membrane anchor.

92. The polynucleotide of any of embodiments 84-91, wherein:

the first nucleic acid encoding CD24 or a biologically active portion encodes the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds to Siglec-10; or

the first nucleic acid encoding CD24 or a biologically active portion encodes the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds to Siglec-10.

93. The polynucleotide of any of embodiments 84-92, wherein the first nucleic acid encoding CD24 comprises the sequence set forth in SEQ ID NO:4 or a sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:4; 94. The polynucleotide of any of embodiments 84-93, wherein the encoded CD47 or biologically active portion binds to SIRPα.

95. The polynucleotide of any of embodiments 84-94, wherein the encoded CD47 or biologically active portion is human. 96. The polynucleotide of any of embodiments 84-95, wherein the encoded CD47 or biologically active portion comprises the extracellular domain or CD47 or a binding portion thereof that binds to SIRPα.

97. The polynucleotide of any of embodiments 84-96, wherein the encoded CD47 or biologically active portion thereof:

(i) comprises the sequence set forth in SEQ ID NO: 7;

(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or

(iii) a binding portion of (i) or (ii) that binds to SIRPα.

98. The polynucleotide of any of embodiments 84-97, wherein the encoded CD47 or biologically active portion thereof comprises the sequence set forth in SEQ ID NO:7.

99. The polynucleotide of any of embodiments 84-97, wherein the encoded CD47 or biologically active portion thereof comprises the sequence set forth in SEQ ID NO:11.

100. The polynucleotide of any of embodiments 84-97 and 99, wherein the encoded CD47 or biologically active portion thereof comprises the sequence set forth in SEQ ID NO:9

101. The polynucleotide of any of embodiments 84-97 and 99, wherein the encoded CD47 or biologically active portion thereof comprises the sequence set forth in SEQ ID NO:10.

102. The polynucleotide of any of embodiments 84-97 and 99, wherein the encoded CD47 or biologically active portion thereof comprises the sequence set forth in SEQ ID NO:12.

103. The polynucleotide of any of embodiments 84-102, wherein the encoded CD47 or biologically active portion comprises a transmembrane domain.

104. The polynucleotide of any of embodiments 84-103, wherein:

the second nucleic acid encodes the sequence set forth in SEQ ID NO: 5 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:5 that binds to SIRPα; or

the second nucleic acid encodes the sequence set forth in SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα.

105. The polynucleotide of any of embodiments 84-104, wherein the second nucleic acid encoding CD47 comprises the sequence set forth in SEQ ID NO:13 or a sequence having at least at or about 90%, at least at or about 91%, at or about 92%, at least at or about 93%, at least at or about 94%, at or about 95%, at least at or about 96%, at least at or about 97%, at least at or about 98%, or at least at or about 99% identity to SEQ ID NO:13.

106. The polynucleotide of any of embodiments 84-105, further comprising at least one promoter that is operatively linked to control expression of the CD24 or biologically active portion and/or the CD47 or biologically active portion.

107. The polynucleotide of any of embodiments 84-106, wherein the first and second nucleic acid are operatively linked to the same promoter.

108. The polynucleotide of any of embodiments 84-106, wherein the first nucleic acid is operatively linked to a first promoter and the second nucleic acid is operatively linked to a second promoter.

109. The polynucleotide of embodiment 108, wherein the first and second promoter are different.

110. The polynucleotide of any of embodiments 106-109, wherein the promoter, or each promoter individually, is a heterologous promoter.

111. The polynucleotide of any of embodiments 106-110, wherein the promoter, or each promoter individually, is an inducible promoter.

112. The polynucleotide of any of embodiments 84-111, further comprising a nucleic acid sequence encoding a linking peptide between the first and second nucleic acid sequences, wherein the linking peptide separates the translation products of the first and second nucleic acid sequences during or after translation.

113. The polynucleotide of embodiment 112, wherein the linking peptide comprises an internal ribosome entry site (IRES), a self-cleaving peptide, or a peptide that causes ribosome skipping, optionally a T2A peptide.

114. A vector, comprising the polynucleotide of any of embodiments 84-112.

115. The vector of embodiment 114, wherein the vector is a mammalian vector, viral vector or artificial chromosome, optionally wherein the artificial chromosome is a bacterial artificial chromosome (BAC).

116. A method of making a non-cell particle comprising CD24 or a biologically active portion, comprising:

a) providing a cell that comprises a nucleic acid encoding CD24 or a biologically active portion thereof;

b) culturing the cell under conditions that allow for production of a non-cell particle, and

c) separating, enriching, or purifying the particle from the cell, thereby making the fusosome.

117. The method of embodiment 116, wherein the cell further comprises a nucleic acid encoding CD47 or a biologically active portion thereof or the nucleic acid further encodes CD47 or a biologically active portion thereof.

118. A method of making a non-cell particle comprising CD24 or a biologically active portion, comprising:

a) providing a cell that comprises the polynucleotide of any of embodiments 84-112 or the vector of embodiment 114 or embodiment 115;

b) culturing the cell under conditions that allow for production of a non-cell particle, and

c) separating, enriching, or purifying the non-cell particle from the cell, thereby making the fusosome.

119. The method of any of embodiments 116-118, wherein the cell is a mammalian cell and the non-cell particle is a vesicle or an exosome, optionally wherein the vesicle is a microvesicle or a nanovesicle.

120. The method of any of embodiments 116-118, wherein the cell is a producer cell and the non-cell particle is a viral particle or a viral-like particle, optionally a retroviral particle or a retroviral-like particle, optionally a lentiviral particle or lentiviral-like particle.

121. The method of any of embodiments 116-120, wherein the cell further comprises an exogenous nucleic acid sequence encoding an exogenous agent.

122. The method of any of embodiments 116-121, wherein the cell further comprises a fusogen.

123. A non-cell particle made by the method of any of embodiments 116-122.

124. A mammalian cell comprising (i) a viral nucleic acid(s) and (ii) nucleic acid encoding an exogenous CD24 or a biologically active portion thereof, optionally wherein the viral nucleic acid(s) are lentiviral nucleic acids.

125. The mammalian cell of embodiment 124, wherein the viral nucleic acid(s) lacks one or more genes involved in viral replication.

126. The mammalian cell of embodiment 124 or embodiment 125, wherein the viral nucleic acid comprises:

one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3);

a nucleic acid encoding a viral envelope protein; and/or

a nucleic acid encoding a viral packaging protein selected from one or more of Gag, Pol, Rev and Tat.

127. The mammalian cell of any of embodiments 124-126, wherein the the exogenous CD24 or biologically active portion comprises:

(i) the sequence set forth in SEQ ID NO: 2;

(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds Siglec-10; or

(iii) a binding portion of (i) or (ii) that binds to Siglec-10.

128. The mammalian cell of embodiment 127, wherein the nucleic acid encoding exogenous CD24 further encodes a a Glycosylphosphatidylinositol (GPI) membrane anchor signal sequence or a transmembrane domain.

129. The mammalian cell of any of embodiments 124-128, wherein the cell further comprises a nucleic acid encoding exogenous CD47 or a biologically active portion.

130. The mammalian cell of embodiment 129, wherein the exogenous CD47 or biologically active portion comprises:

(i) the sequence set forth in SEQ ID NO: 7;

(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or

(iii) a binding portion of (i) or (ii) that binds to SIRPα.

131. The mammalian cell of embodiment 130, wherein the nucleic acid encoding exogenous CD47 further encodes a Glycosylphosphatidylinositol (GPI) membrane anchor signal sequence or a transmembrane domain.

132. The mammalian cell of any of embodiments 124-131, wherein the nucleic acid encoding the exogenous CD24 or biologically active portion and the nucleic acid encoding the exogenous CD47 or biologically active portion are encoded by the polynucleotide of any of embodiments 84-112.

133. A viral vector particle or viral-like particle produced from the mammalian cell of any of embodiments 124-132.

134. A composition comprising a plurality of non-cell particles of any of embodiments 1-45.

135. A composition comprising a plurality of pseudotyped lentivirus or lentiviral-like particles of any of embodiments 46-81.

136. The composition of embodiment 134 or embodiment 135 further comprising a pharmaceutically acceptable carrier.

137. The pharmaceutical composition of any of embodiments 134-136, wherein the plurality of particles comprise an average diameter of less than 1 μm.

138. A method of delivering an exogenous agent to a subject (e.g., a human subject), the method comprising administering to the subject the non-cell particle of any of embodiments 1-45 or the pseudotyped lentivirus or lentiviral-like particles of any of embodiments 46-81 or the composition of any of embodiments 134-137.

139. A method of treating a disease or disorder in a subject (e.g., a human subject), the method comprising administering to the subject a non-cell particle of any of embodiments 1-45 or the pseudotyped lentivirus or lentiviral-like particles of any of embodiments 46-81 or the composition of any of embodiments 134-137.

140. A method of evading phagocytosis of a particle by a phagocytic cell, the method comprising contacting a phagocytic cell with a non-cell particle of any of embodiments 1-45 or the pseudotyped lentivirus or lentiviral-like particles of any of embodiments 46-81 or the composition of any of embodiments 134-137, whereby said particles evades phacocytosis by said phagocytic cell.

141. A method of increasing the life of a particle in vivo in a mammal, method comprising administering the non-cell particle of any of embodiments 1-45 or the pseudotyped lentivirus or lentiviral-like particles of any of embodiments 46-81 or the composition of any of embodiments 134-137 to a mammalian subject wherein said administered particles have a longer half-life in said mammal than an otherwise similar particle that does not have CD24 expressed thereon.

142. The non-cell particle of any of embodiments 1-27, 29, 30, and 32-45, further comprising an exogenous agent.

143. The non-cell particle of any of embodiments 28, 31, 82, 83, and 142, wherein the exogenous agent comprises a protein.

144. The non-cell particle of any of embodiments 28, 31, 82, 83, 142, and 143, wherein the exogenous agent comprises a membrane protein.

145. The non-cell particle of any of embodiments 28, 31, 82, 83, and 142-144, wherein the exogenous agent comprises a chimeric antigen receptor (CAR), a T cell receptor, an integrin, an ion channel, a pore forming protein, a Toll-Like Receptor, an interleukin receptor, a cell adhesion protein, or a transport protein.

146. The non-cell particle of any of embodiments 28, 31, 82, 83, and 142-145, wherein the exogenous agent comprises a CAR comprising an antigen binding domain, a transmembrane domain, and one or more signaling domains.

147. The non-cell particle of embodiment 146, wherein the antigen binding domain binds to a surface antigen characteristic of a cell type or a disorder.

148. The non-cell particle of embodiment 146 or 147, wherein the antigen binding domain binds to a surface antigen characteristic of a neoplastic cell, a T cell, an autoimmune or inflammatory disorder, a senescent cell, or an infectious disease.

149. A method of delivering an exogenous agent to a subject (e.g., a human subject), the method comprising administering to a subject the non-cell particle of any of embodiments 28, 31, 82, 83, and 142-148, wherein the payload gene encoding the exogenous agent or the exogenous agent is delivered to a target cell.

150. The method of embodiment 149, wherein the exogenous agent is a CAR.

151. The method of embodiment 149 or embodiment 150, wherein the target cell is a T cell.

152. The method of any of embodiments 149-151, wherein the target cell is any of a CD4+T cell, a CD8+T cell, an alpha beta T cell, a gamma delta T cell, a naive T cell, an effector T cell, a cytotoxic T cell (e.g., a CD8+ cytotoxic T cell), a regulatory T cell (e.g., a thymus-derived regulatory T cell, a peripherally derived regulatory T cell, a CD4+Foxp3+ regulatory T cell, or a CD4+FoxP3− type 1 regulatory T (Tr1) cell), a helper T cell (e.g., a CD4+ helper T cell, a Th1 cell, a Th2 cell, a Th3 cell, a Th9 cell, a Th17 cell, a Th22 cell, or a T follicular helper (Tfh) cell), a memory T cell (e.g., a stem cell memory T cell, a central memory T cell, or an effector memory T cell), a NKT cell, and a Mucosal associated invariant T (MAIT) cell.

### X. EXAMPLES

The following examples are included for illustrative purposes only and are not intended to limit the scope of the invention.

### Example 1: Modification of Retroviral Vector with CD24 to Evade Macrophage Phagocytosis

This Example describes quantification of the evasion of phagocytosis by a modified retroviral vector. In an embodiment, a modified retroviral vector will evade phagocytosis by macrophages.

Cells engage in phagocytosis, engulfing particles, enabling the sequestration and destruction of foreign invaders, like bacteria or dead cells. In some embodiments, phagocytosis of lentiviral vectors by macrophages would reduce their activity. In some embodiments, phagocytosis of lentiviral vectors is a measure of immunogenicity of retroviral vectors.

Retroviral vectors are produced from cells which lack CD24 (hereinafter NMC, positive control), cells that are transfected with CD24 cDNA (hereinafter NMC-CD47), and cells transfected with an empty vector control (hereinafter NMC-empty vector, negative control). Prior to retroviral vector production, the cells are labeled with CSFE.

Reduction of macrophage mediated immune clearance is determined with a phagocytosis assay according to the following protocol. Macrophages are plated immediately after harvest in confocal glass bottom dishes. Macrophages are incubated in DMEM+10% FBS+1% P/S for 1h to attach. An appropriate number of retroviral vectors produced from NMC, NMC-CD24, NMC-empty vector are added to the macrophages as indicated in the protocol, and are incubated for 2h, substantially as described in tools.thermofisher.com/content/sfs/manuals/mp06694.pdf.

After 2h, the dish is gently washed, and intracellular fluorescence is examined. Intracellular fluorescence emitted by engulfed retroviral particles is imaged by confocal microscopy at 488 excitation. The total fluorescence and/or number of phagocytotic positive macrophage is quantified using imaging software. The data may be expressed as the phagocytic index=(number of macrophages containing engulfed cells/total number of counted macrophages)×100.

In an embodiment, the phagocytic index will be reduced when macrophages are incubated with retroviral vectors derived from NMC-CD24, versus those derived from NMC, or NMC-empty vector.

### Example 2: Modification of Retroviral Vector with CD24 and CD47 to Evade Macrophage Phagocytosis

This Example describes quantification of the evasion of phagocytosis by a modified retroviral vector. In an embodiment, a modified retroviral vector will evade phagocytosis by macrophages.

Cells engage in phagocytosis, engulfing particles, enabling the sequestration and destruction of foreign invaders, like bacteria or dead cells. In some embodiments, phagocytosis of lentiviral vectors by macrophages would reduce their activity. In some embodiments, phagocytosis of lentiviral vectors is a measure of immunogenicity of retroviral vectors.

Retroviral vectors are produced from cells which lack CD24 and CD47 (hereinafter NMC, positive control), cells that are transfected with CD24 and CD47 cDNA (hereinafter NMC-CD24-CD47), and cells transfected with an empty vector control (hereinafter NMC-empty vector, negative control). Prior to retroviral vector production, the cells are labeled with CSFE.

Reduction of macrophage mediated immune clearance is determined with a phagocytosis assay according to the following protocol. Macrophages are plated immediately after harvest in confocal glass bottom dishes. Macrophages are incubated in DMEM+10% FBS+1% P/S for 1h to attach. An appropriate number of retroviral vectors produced from NMC, NMC-CD24-CD47, NMC-empty vector are added to the macrophages as indicated in the protocol, and are incubated for 2h, substantially as described in tools.thermofisher.com/content/sfs/manuals/mp06694.pdf.

After 2h, the dish is gently washed, and intracellular fluorescence is examined. Intracellular fluorescence emitted by engulfed retroviral particles is imaged by confocal microscopy at 488 excitation. The total fluorescence and/or number of phagocytotic positive macrophage is quantified using imaging software. The data may be expressed as the phagocytic index=(number of macrophages containing engulfed cells/total number of counted macrophages)×100.

In an embodiment, the phagocytic index will be reduced when macrophages are incubated with retroviral vectors derived from NMC-CD24-CD48, versus those derived from NMC, or NMC-empty vector.

### Example 3: Production of LV Particles with Human CD24 and/or Human CD47

Laboratory-grade VSV-G-pseudotyped third-generation SIN lentiviral vectors (LVs) are produced by calcium phosphate transient transfection into 293T cells or by LV stable producer cell lines. 293T cells are transfected with a solution containing third generation lentiviral packaging plasmids expressing (i) gag and pol, (ii) rev, and (iii) VSV-G envelope protein, along with the expression plasmids pCMV-hCD47 and/or pCMV-hCD24, and transgene transfer plasmid pLenti-GFP. The amount of human CD47 and human CD24 plasmids are varied to generate LV particles with variable CD47 and/or CD24. Medium is changed 14 to 16 hours after transfection, and supernatant is collected 30 hours after medium change. LV-containing supernatants are sterilized through a 0.22-μm filter and, as necessary is transferred into sterile polyal-lomer tubes and centrifuged at 20,000g for 120 minutes at 20° C. (Beckman Optima XL-100 K Ultracentrifuge). LV pellets are dissolved in the appropriate volume of Phosphate Buffered Saline (PBS) to allow 500× to 1000× concentrations.

For LV titration, 1e5 293T cells are transduced with serial LV dilutions in the presence of polybrene (8 μg/ml). For LV-GFP, cells are analyzed by flow cytometry 3-7 days after transduction and infectious titer, expressed as transducing units (TU)/mL, is calculated using the formula TU/mL=((% GFP+ cells/100)×100,000×(1/dilution factor)).

LVs are analyzed for CD24 or CD47 expression by immunoblotting. To reduce and denature LV samples, each sample is boiled in sample buffer at 100° C. for 5 minutes. Sodium dodecyl sulphate polyacrylamide gel electrophoresis (SDS-PAGE) is used to resolve proteins from LV particles. Resolved gels are transferred to Polyvinylidene difluoride (PVDF) membrane by immersion tank electrophoresis. Antibodies for human CD47, human CD24, and p24 are used to identify amounts of proteins present in LV preparations. Ratios of CD47 and CD24 to p24 provide a qualitative measure of average CD47 or CD24 per LV particle. This can then be related to the plasmid concentration used during LV preparation.

Increased amounts of CD24 and CD47 plasmid during transfection increases LV incorporation of these proteins as detected by immunoblotting.

### Example 4: Analysis of CD24 and/or CD47 LV Uptake into Human Macrophages

LVs with CD47 or CD24 are produced as described in Example 3.

Leukocyte reduction system (LRS) chambers from anonymous donors are obtained from local blood banks. Peripheral monocytes are purified through successive density gradients using Ficoll (Sigma Aldrich) and Percoll (GE Healthcare). Monocytes are then differentiated into macrophages by 7-9 days of culture in IMDM+10% AB human serum. Unless otherwise stated, macrophages used for all in vitro phagocytosis assays are stimulated with 50 ng/mL human TGFβ1 and 50 ng/mL human IL-10 on Days 3-4 of differentiation until the end of the culture period on Days 7-9. IL-4 stimulation is added at a concentration of 20 ng/mL on Days 3-4 of differentiation until the end of the culture period on Days 7-9.

In vitro phagocytosis assays are performed by incubating 100,000 donor-derived macrophages with serial dilutions of crude LV preparations (1:2 thru 1:10000 by volumetric ratio) overnight in a humidified, 5% CO₂ incubator at 37° C. in ultra-low-attachment 96-well U-bottom plates in serum-free IMDM (Life Technologies). Plates are centrifuged and medium is changed after overnight incubation. Macrophages are incubated for another 3 days to permit lentiviral expression before being harvested for flow cytometry.

Macrophages are harvested by centrifugation at 400 g for 5 minutes at 4° C. and stained with A647-labeled anti-CD11b to identify human macrophages. Assays are analyzed by flow cytometry on an LRSFortessa Analyzer (BD Biosciences) using a high-throughput auto-sampler. Phagocytosis is measured as the number of CD11b+, GFP+ macrophages, quantified as a percentage of the total CD11b+ macrophages.

GFP+ macrophages due to LV-mediated transduction/uptake are reduced in CD47 and CD24 expressing LVs and further reduced in LVs with both CD47 and CD24. Moreover, GFP+ macrophage numbers decrease with increasing CD47 and/or CD24 levels on LV particles.

### Example 5: Reduction of In Vivo CD24 and/or CD47 LV Uptake into Mouse Macrophages

LVs with CD47 or CD24 are produced as described in Examples 1 and 3, with the substitution of the use of pCMV-mCD47 and pCMV-mCD24 for transfection. Analysis of CD47 and CD24 incorporation is carried out using immunoblotting with anti-mouse CD47 and anti-mouse CD24 antibodies as is described in Example 3.

Vector administration is carried out in adult (7 to 10 weeks old) wild-type C57BL/6 mice by tail-vein injection. Mice are bled from the retro-orbital plexus using capillary tubes, and blood is collected into 0.38% sodium citrate buffer (pH 7.4). Mice are anesthetized with tribromoethanol and euthanized by C02 inhalation at scheduled times following LV administration. All animal procedures are performed according to protocols approved by the Institutional Animal Care and Use Committee.

LVs are delivered at time zero (TO) to five cohorts of mice (N=5 each). Blood is sampled at 5 minutes, 10 minutes, 30 minutes, 1 hour, 2 hour, 4 hours, 8 hours, 12 hours, 24 hours, 48 hours rotating through the five cohorts to prevent undue blood volume depletion of the mice.

Blood samples are analyzed for viral RNA. RNA extraction is performed using the RNeasy Plus mini Kit (Qiagen) according to manufacturer's instructions and reverse transcribed using the SuperScript Vilo kit. All q-PCR analyses are performed using TaqMan probes from Applied Biosystems (WPRE: primer fw 5′-GGCTGTTGGGCACTGACAAT-3′; primer rv 5′-ACGTCCCGCGCAGAATC-3′; probe FAM 5′-TTTCCTTGGCTGCTCGCCTGTGT-3′ NGB).

Q-PCR is run for 40 cycles using the Viia 7 instrument and raw data (Ct) are analyzed as follows: to determine gene expression, the difference (ΔCt) between the threshold cycle (Ct) of WPRE and that of the reference gene TAF7 is calculated by applying an equal threshold. The lower the ΔCt, the higher the gene expression level.

The time course of viral RNA in serum shows a decrease over time. The addition of CD47 and/or CD24 reduces the rate of decrease, indicating an increase in viral vector bioavailability. Increasing levels of CD47 and/or CD24 reduces the rate of decrease in a dose-dependent manner.

The present invention is not intended to be limited in scope to the particular disclosed embodiments, which are provided, for example, to illustrate various aspects of the invention.

Various modifications to the compositions and methods described will become apparent from the description and teachings herein. Such variations may be practiced without departing from the true scope and spirit of the disclosure and are intended to fall within the scope of the present disclosure.

### XI. SEQUENCES

| # | SEQUENCE | Description |
|---|---|---|
| 1 | MGRAMVARLG LGLLLLALLL PTQIYSSETT TGTSS NSSQS | CD24 full |
| | TSNSGLAPNP TNATTKAAGG ALQSTASLFV VSLSLLHLYS | human aa |
| 2 | SETT TGTSSNSSQS TSNSGLAPNP TNATTKAA | CD 24 |
| | | extracellular |
| | | domain |
| | | human aa |
| 3 | SETT TGTSSNSSQS TSNSGLAPNP TNATTKAAGG | CD24 Mature |
| | ALQSTASLFV VSLSLLHLYS | human aa |
| 4 | ATGGGCAGAG CAATGGTGGC CAGGCTCGGG CTGGGGCTGC | CD24 human |
| | TGCTGCTGGC ACTGCTCCTA CCCACGCAGA TTTATTCCAG | full nt |
| | TGAAACAACA ACTGGAACTT CAAGTAACTC CTCCCAGAGT | |
| | ACTTCCAACT CTGGGTTGGC CCCAAATCCA ACTAATGCCA | |
| | CCACCAAGGC GGCTGGTGGT GCCCTGCAGT CAACAGCCAG | |
| | TCTCTTCGTG GTCTCACTCT CTCTTCTGCA TCTCTACTCT | |
| | TAA | |
| 5 | MWPLVAALLLGSACCGSAQLLFNKTKSVEFTFCNDTVVIPCFV | CD47 human |
| | TNMEAQNTTEVYVKWKFKGRDIYTFDGALNKSTVPTDFSSAKI | full aa |
| | EVSQLLKGDASLKMDKSDAVSHTGNYTCEVTELTREGETIIELK | |
| | YRVVSWFSPNENILIVIFPIFAILLFWGQFGIKTLKYRSGGMDEK | |
| | TIALLVAGLVITVIVIVGAILFVPGEYSLKNATGLGLIVTSTGILIL | |
| | LHYYVFSTAIGLTSFVIAILVIQVIAYILAVVGLSLCTAACIPMHG | |
| | PLLISGLSILALAQLLGLVYMKFV | |
| 6 | MWPLVAALLLGSACCGSAQLLFNKTKSVEFTFCNDTVVIPCFV | CD47 |
| | TNMEAQNTTEVYVKWKFKGRDIYTFDGALNKSTVPTDFSSAKI | extracellular |
| | EVSQLLKGDASLKMDKSDAVSHTGNYTCEVTELTREGETIIEL | domain |
| | KYRVVSWFSPN | human aa |
| 7 | QLLFNKTKSVEFTFCNDTVVIPCFVTNMEAQNTTEVYVKWKFK | CD47 mature |
| | GRDIYTFDGALNKSTVPTDFSSAKIEVSQLLKGDASLKMDKSDA | extracellular |
| | VSHTGNYTCEVTELTREGETIIELKYRVVSWFSPN | domain |
| | | human aa |
| 8 | QLLFNKTKSVEFTFCNDTVVIPCFVTNMEAQNTTEVYVKWKFK | CD47 |
| | GRDIYTFDGALNKSTVPTDFSSAKIEVSQLLKGDASLKMDKSDA | mature |
| | VSHTGNYTCEVTELTREGETIIELKYRVVSWFSPNENILIVIFPIFA | human aa |
| | ILLFWGQFGIKTLKYRSGGMDEKTIALLVAGLVITVIVIVGAILF | |
| | VPGEYSLKNATGLGLIVTSTGILILLHYYVI STAIGLTSE VIAILVI | |
| | QVIAYI | |
| | LAVVGLSLCIAACIPMHGPLLISGLSILALAQLLGLVYMKFV | |
| 9 | GNYTCEVTELTREGETIIELK | CD47 peptide |
| 10 | XAA-EVTELTREGE-XAA | CD47 peptide |
| | XAA IS C, T OR NULL | |
| 11 | EVTELTREGE | CD47 peptide |
| 12 | CEVTELTREGEC | CD47 peptide |
| 13 | ATGTGGCCCC TGGTAGCGGC GCTGTTGCTG GGCTCGGCGT | CD47 full |
| | GCTGCGGATC AGCTCAGCTA CTATTTAATA AAACAAAATC | human nt |
| | TGTAGAATTC ACGTTTTGTA ATGACACTGT CGTCATTCCA | |
| | TGCTTTGTTA CTAATATGGA GGCACAAAAC ACTACTGAAG | |
| | TATACGTAAA GTGGAAATTT AAAGGAAGAG ATATTTACAC | |
| | CTTTGATGGA GCTCTAAACA AGTCCACTGT CCCCACTGAC | |
| | TTTAGTAGTG CAAAAATTGA AGTCTCACAA TTACTAAAAG | |
| | GAGATGCCTC TTTGAAGATG GATAAGAGTG ATGCTGTCTC | |
| | ACACACAGGA AAGTAGACTT GTGAAGTAAC AGAATTAACC | |
| | AGAGAAGGTG AAACGATCAT CGAGCTAAAA TATCGTGTTG | |
| | TTTCATGGTT TTCTCCAAAT GAAAATATTC TTATTGTTAT | |
| | TTTCCCAATT TTTGCTATAC TCCTGTTCTG GGGACAGTTT | |
| | GGTATTAAAA CACTTAAATA TAGATCCGGT GGTATGGATG | |
| | AGAAAACAAT TGCTTTACTT GTTGCTGGAC TAGTGATCAC | |
| | TGTCATTGTC ATTGTTGGAG CCATTCTTTT CGTCCCAGGT | |
| | GAATATTCAT TAAAGAATGC TACTGGCCTT GGTTTAATTG | |
| | TGACTTCTAC AGGGATATTA ATATTACTTC ACTACTATGT | |
| | GTTTAGTACA GCGATTGGAT TAACCTCCTT CGTCATTGCC | |
| | ATATTGGTTA TTCAGGTGAT AGCCTATATC CTCGCTGTGG | |
| | TTGGACTGAG TCTCTGTATT GCGGCGTGTA TACCAATGCA | |
| | TGGCCCTCTT CTGATTTCAG GTTTGAGTAT CTTAGCTCTA | |
| | GCACAATTAC TTGGACTAGT TTATATGAAA TTTGTGGAAT | |
| | AA | |
| 14 | NILIVIFPIFAILLFWGQFGIKTLKYRSGGMDEKTIALLVAGLVIT | Human CD47 |
| | VIVIVGAILFVPGEYSLKNATGLGLIVTSTGILILLHYYVFSTAIG | transmembra |
| | LTSFVIA | nedomain |
| | ILVIQVIAYILAVVGLSLCIAACIPMHGPLLISGLSILALAQLLGLV | |
| | YM | |
| 15 | MVGRFCPESPPGFVRVAATSAVSLDPPSGEPRPGCGYPGPRSAA | Human CD24 |
| | SRVYGCTAPARETGGWAWETLAGAGAKKIYSSETTTGTSSNSS | |
| | QSTSNSGLAPNPTNATTKAAGGALQSTASLFVVSLSLLHLYS | |
| 16 | MVGRFCPESPPGFVQVAATSAVSLDPPSGEPRPGCGYLGPRSAA | Bonobo |
| | SRVYGCTAPARETGGWAWETLAGVGAKKIYSSETTTGTSSNSS | CD24 |
| | QSTSNSGFAPNPTNATTKAAGGALQSTASLFVVSLSLLHLYS | |
| 17 | MVGRFCPESPPGFVQVAATSAVSLDPPSGEPRPGCGYLGPRSAA | Chimp CD24 |
| | SRVYGCTAPARETGGWAWETLAGVGAKKIYSSETTTGTSSNSS | |
| | QSTSNSGFAPNPTNATTKAAGGALQSTASLFVVSLSLLHLYS | |
| 18 | SETT TGTSSNSSQS TSNSGFAPNP TNATTKA A | CD 24 |
| | | extracellular |
| | | domain aa |
| 19 | MGPAENKKVR FENTTSDKGK IPSKVIKSYY GTMDIKKINE | NiVG protein |
| | GLLDSKILSA FNTVIALLGS IVIIVMNIMI IQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | (602 aa) |
| | ISCPNPLPFR | |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 20 | MMADSKLVSLNNNLSGKIKDQGKVIKNYYGTMDIKKINDGLLDSKIL | Hendra Virus |
| | GAFNTVIALLGSIIIIVMNIMIIQNYTRTTDNQALIKESLQSVQQQIKALT | G Protein |
| | DKIGTEIGPKVSLIDTSSTITIPANIGLLGSKISQSTSSINENVNDKCKFTL | |
| | PPLKIHECNISCPNPLPFREYRPISQGVSDLVGLPNQICLQKTTSTILKPR | |
| | LISYTLPINTREGVCITDPLLAVDNGFFAYSHLEKIGSCTRGIAKQRIIG | |
| | VGEVLDRGDKVPSMFMTNVWTPPNPSTIHHCSSTYHEDFYYTLCAVS | |
| | HVGDPILNSTSWTESLSLIRLAVRPKSDSGDYNQKYIAITKVERGKYD | |
| | KVMPYGPSGIKQGDTLYFPAVGFLPRTEFQYNDSNCPIIHCKYSKAEN | |
| | CRLSMGVNSKSHYILRSGLLKYNLSLGGDIILQFIEIADNRLTIGSPSKI | |
| | YNSLGQPVFYQASYSWDTMIKLGDVDTVDPLRVQWRNNSVISRPGQS | |
| | QCPRFNVCPEVCWEGTYNDAFLIDRLNWVSAGVYLNSNQTAENPVF | |
| | AVFKDNEILYQVPLAEDDTNAQKTITDCFLLENVIWCISLVEIYDTGDS | |
| | VIRPKLFAVKIPAQCSES | |
| 21 | MADSKLVSLNNNLSGKIKDQGKVIKNYYGTMDIKKINDGLLDSKILG | Hendra Virus |
| | AFNTVIALLGSIIIIVMNIMIIQNYTRTTDNQALIKESLQSVQQQIKALTD | G Protein |
| | KIGTEIGPKVSLIDTSSTITIPANIGLLGSKISQSTSSINENVNDKCKFTLP | without Met |
| | PLKIHECNISCPNPLPFREYRPISQGVSDLVGLPNQICLQKTTSTILKPRL | |
| | ISYTLPINTREGVCITDPLLAVDNGFFAYSHLEKIGSCTRGIAKQRIIGV | |
| | GEVLDRGDKVPSMFMTNVWTPPNPSTIHHCSSTYHEDFYYTLCAVSH | |
| | VGDPILNSTSWTESLSLIRLAVRPKSDSGDYNQKYIAITKVERGKYDK | |
| | VMPYGPSGIKQGDTLYFPAVGFLPRTEFQYNDSNCPIIHCKYSKAENC | |
| | RLSMGVNSKSHYILRSGLLKYNLSLGGDIILQFIEIADNRLTIGSPSKIY | |
| | NSLGQPVFYQASYSWDTMIKLGDVDTVDPLRVQWRNNSVISRPGQSQ | |
| | CPRFNVCPEVCWEGTYNDAFLIDRLNWVSAGVYLNSNQTAENPVFA | |
| | VFKDNEILYQVPLAEDDTNAQKTITDCFLLENVIWCISLVEIYDTGDSV | |
| | IRPKLFAVKIPAQCSES | |
| 22 | MPAENKKVRFENTTSDKGKIPSKVIKSYYGTMDIKKINEGLLDSKILSA | Nipah Virus G |
| | FNTVIALLGSIVIIVMNIMIIQNYTRSTDNQAVIKDALQGIQQQIKGLAD | Protein |
| | KIGTEIGPKVSLIDTSSTITIPANIGLLGSKISQSTASINENVNEKCKFTLP | |
| | PLKIHECNISCPNPLPFREYRPQTEGVSNLVGLPNNICLQKTSNQILKPK | |
| | LISYTLPVVGQSGTCITDPLLAMDEGYFAYSHLERIGSCSRGVSKQRII | |
| | GVGEVLDRGDEVPSLFMTNVWTPPNPNTVYHCSAVYNNEFYYVLCA | |
| | VSTVGDPILNSTYWSGSLMMTRLAVKPKSNGGGYNQHQLALRSIEKG | |
| | RYDKVMPYGPSGIKQGDTLYFPAVGFLVRTEFKYNDSNCPITKCQYS | |
| | KPENCRLSMGIRPNSHYILRSGLLKYNLSDGENPKVVFIEISDQRLSIGS | |
| | PSKIYDSLGQPVFYQASFSWDTMIKFGDVLTVNPLVVNWRNNTVISRP | |
| | GQSQCPRFNTCPEICWEGVYNDAFLIDRINWISAGVFLDSNQTAENPV | |
| | FTVFKDNEILYRAQLASEDTNAQKTITNCFLLKNKIWCISLVEIYDTGD | |
| | NVIRPKLFAVKIPEQCT | |
| 23 | PAENKKVRFENTTSDKGKIPSKVIKSYYGTMDIKKINEGLLDSKILSAF | Nipah Virus G |
| | NTVIALLGSIVIIVMNIMIIQNYTRSTDNQAVIKDALQGIQQQIKGLADK | Protein (No |
| | IGTEIGPKVSLIDTSSTITIPANIGLLGSKISQSTASINENVNEKCKFTLPP | Met) |
| | LKIHECNISCPNPLPFREYRPQTEGVSNLVGLPNNICLQKTSNQILKPKL | |
| | ISYTLPVVGQSGTCITDPLLAMDEGYFAYSHLERIGSCSRGVSKQRIIG | |
| | VGEVLDRGDEVPSLFMTNVWTPPNPNTVYHCSAVYNNEFYYVLCAV | |
| | STVGDPILNSTYWSGSLMMTRLAVKPKSNGGGYNQHQLALRSIEKGR | |
| | YDKVMPYGPSGIKQGDTLYFPAVGFLVRTEFKYNDSNCPITKCQYSKP | |
| | ENCRLSMGIRPNSHYILRSGLLKYNLSDGENPKVVFIEISDQRLSIGSPS | |
| | KIYDSLGQPVFYQASFSWDTMIKFGDVLTVNPLVVNWRNNTVISRPG | |
| | QSQCPRFNTCPEICWEGVYNDAFLIDRINWISAGVFLDSNQTAENPVF | |
| | TVFKDNEILYRAQLASEDTNAQKTITNCFLLKNKIWCISLVEIYDTGDN | |
| | VIRPKLFAVKIPEQCT | |
| 24 | MLSQLQKNYLDNSNQQGDKMNNPDKKLSVNFNPLELDKGQKDLNKSYYV | Cedar Virus G |
| | KNKNYNVSNLLNESLHDIKFCIYCIFSLLIIITIINIITISIVITRLKV | Protein |
| | HEENNGMESPNLQSIQDSLSSLTNMINTEITPRIGILVTATSVTLSSSINY | |
| | VGTKTNQLVNELKDYITKSCGFKVPELKLHECNISCADPKISKSAMYS | |
| | TNAYAELAGPPKIFCKSVSKDPDFRLKQIDYVIPVQQDRSICMNNPLL | |
| | DISDGFFTYIHYEGINSCKKSDSFKVLLSHGEIVDRGDYRPSLYLLSSH | |
| | YHPYSMQVINCVPVTCNQSSFVFCHISNNTKTLDNSDYSSDEYYITYF | |
| | NGIDRPKTKKIPINNMTADNRYIHFTFSGGGGVCLGEEFIIPVTTVINTD | |
| | VFTHDYCESFNCSVQTGKSLKEICSESLRSPTNSSRYNLNGIMIISQNN | |
| | MTDFKIQLNGITYNKLSFGSPGRLSKTLGQVLYYQSSMSWDTYLKAG | |
| | FVEKWKPFTPNWMNNTVISRPNQGNCPRYHKCPEICYGGTYNDIAPL | |
| | DLGKDMYVSVILDSDQLAENPEITVFNSTTILYKERVSKDELNTRSTTT | |
| | SCFLFLDEPWCISVLETNRFNGKSIRPEIYSYKIPKYC | |
| 25 | LSQLQKNYLDNSNQQGDKMNNPDKKLSVNFNPLELDKGQKDLNKSYYVKN | Cedar Virus G |
| | KNYNVSNLLNESLHDIKFCIYCIFSLLIIITIINIITISIVITRLKVHEE | Protein (No |
| | NNGMESPNLQSIQDSLSSLTNMINTEITPRIGILVTATSVTLSSSINYVGT | Met) |
| | KTNQLVNELKDYITKSCGFKVPELKLHECNISCADPKISKSAMYSTNA | |
| | YAELAGPPKIFCKSVSKDPDFRLKQIDYVIPVQQDRSICMNNPLLDISD | |
| | GFFTYIHYEGINSCKKSDSFKVLLSHGEIVDRGDYRPSLYLLSSHYHPY | |
| | SMQVINCVPVTCNQSSFVFCHISNNTKTLDNSDYSSDEYYITYFNGIDR | |
| | PKTKKIPINNMTADNRYIHFTFSGGGGVCLGEEFIIPVTTVINTDVFTHD | |
| | YCESFNCSVQTGKSLKEICSESLRSPTNSSRYNLNGIMIISQNNMTDFKI | |
| | QLNGITYNKLSFGSPGRLSKTLGQVLYYQSSMSWDTYLKAGFVEKW | |
| | KPFTPNWMNNTVISRPNQGNCPRYHKCPEICYGGTYNDIAPLDLGKD | |
| | MYVSVILDSDQLAENPEITVFNSTTILYKERVSKDELNTRSTTTSCFLFL | |
| | DEPWCISVLETNRFNGKSIRPEIYSYKIPKYC | |
| 26 | MPQKTVEFINMNSPLERGVSTLSDKKTLNQSKITKQGYFGLGSHSERN | Bat |
| | WKKQKNQNDHYMTVSTMILEILVVLGIMFNLIVLTMVYYQNDNINQ | Paramyxovirus |
| | RMAELTSNITVLNLNLNQLTNKIQREIIPRITLIDTATTITIPSAITYILAT | G Protein |
| | LTTRISELLPSINQKCEFKTPTLVLNDCRINCTPPLNPSDGVKMSSLATN | |
| | LVAHGPSPCRNFSSVPTIYYYRIPGLYNRTALDERCILNPRLTISSTKFA | |
| | YVHSEYDKNCTRGFKYYELMTFGEILEGPEKEPRMFSRSFYSPTNAVN | |
| | YHSCTPIVTVNEGYFLCLECTSSDPLYKANLSNSTFHLVILRHNKDEKI | |
| | VSMPSFNLSTDQEYVQIIPAEGGGTAESGNLYFPCIGRLLHKRVTHPLC | |
| | KKSNCSRTDDESCLKSYYNQGSPQHQVVNCLIRIRNAQRDNPTWDVI | |
| | TVDLTNTYPGSRSRIFGSFSKPMLYQSSVSWHTLLQVAEITDLDKYQL | |
| | DWLDTPYISRPGGSECPFGNYCPTVCWEGTYNDVYSLTPNNDLFVTV | |
| | YLKSEQVAENPYFAIFSRDQILKEFPLDAWISSARTTTISCFMFNNEIW | |
| | CIAALEITRLNDDIIRPIYYSFWLPTDCRTPYPHTGKMTRVPLRSTYNY | |
| 27 | PQKTVEFINMNSPLERGVSTLSDKKTLNQSKITKQGYFGLGSHSERNW | Bat |
| | KKQKNQNDHYMTVSTMILEILVVLGIMFNLIVLTMVYYQNDNINQRMA | Paramyxovirus |
| | ELTSNITVLNLNLNQLTNKIQREIIPRITLIDTATTITIPSAITYILATL | G Protein (No |
| | TTRISELLPSINQKCEFKTPTLVLNDCRINCTPPLNPSDGVKMSSLATNL | Met) |
| | VAHGPSPCRNFSSVPTIYYYRIPGLYNRTALDERCILNPRLTISSTKFAY | |
| | VHSEYDKNCTRGFKYYELMTFGEILEGPEKEPRMFSRSFYSPTNAVNY | |
| | HSCTPIVTVNEGYFLCLECTSSDPLYKANLSNSTFHLVILRHNKDEKIV | |
| | SMPSFNLSTDQEYVQIIPAEGGGTAESGNLYFPCIGRLLHKRVTHPLCK | |
| | KSNCSRTDDESCLKSYYNQGSPQHQVVNCLIRIRNAQRDNPTWDVIT | |
| | VDLTNTYPGSRSRIFGSFSKPMLYQSSVSWHTLLQVAEITDLDKYQLD | |
| | WLDTPYISRPGGSECPFGNYCPTVCWEGTYNDVYSLTPNNDLFVTVY | |
| | LKSEQVAENPYFAIFSRDQILKEFPLDAWISSARTTTISCFMFNNEIWCI | |
| | AALEITRLNDDIIRPIYYSFWLPTDCRTPYPHTGKMTRVPLRSTYNY | |
| 28 | MATNRDNTITSAEVSQEDKVKKYYGVETAEKVADSISGNKVFILMNT | Mojiang virus, |
| | LLILTGAIITITLNITNLTAAKSQQNMLKIIQDDVNAKLEMFVNLDQLV | Tongguan 1 G |
| | KGEIKPKVSLINTAVSVSIPGQISNLQTKFLQKYVYLEESITKQCTCNPL | Protein |
| | SGIFPTSGPTYPPTDKPDDDTTDDDKVDTTIKPIEYPKPDGCNRTGDHF | |
| | TMEPGANFYTVPNLGPASSNSDECYTNPSFSIGSSIYMFSQEIRKTDCT | |
| | AGEILSIQIVLGRIVDKGQQGPQASPLLVWAVPNPKIINSCAVAAGDE | |
| | MGWVLCSVTLTAASGEPIPHMFDGFWLYKLEPDTEVVSYRITGYAYL | |
| | LDKQYDSVFIGKGGGIQKGNDLYFQMYGLSRNRQSFKALCEHGSCLG | |
| | TGGGGYQVLCDRAVMSFGSEESLITNAYLKVNDLASGKPVIIGQTFPP | |
| | SDSYKGSNGRMYTIGDKYGLYLAPSSWNRYLRFGITPDISVRSTTWLK | |
| | SQDPIMKILSTCTNTDRDMCPEICNTRGYQDIFPLSEDSEYYTYIGITPN | |
| | NGGTKNFVAVRDSDGHIASIDILQNYYSITSATISCFMYKDEIWCIAITE | |
| | GKKQKDNPQRIYAHSYKIRQMCYNMKSATVTVGNAKNITIRRY | |
| 29 | ATNRDNTITSAEVSQEDKVKKYYGVETAEKVADSISGNKVFILMNTL | Mojiang virus, |
| | LILTGAIITITLNITNLTAAKSQQNMLKIIQDDVNAKLEMFVNLDQLVK | Tongguan 1 G |
| | GEIKPKVSLINTAVSVSIPGQISNLQTKFLQKYVYLEESITKQCTCNPLS | (No Met) |
| | GIFPTSGPTYPPTDKPDDDTTDDDKVDTTIKPIEYPKPDGCNRTGDHFT | |
| | MEPGANFYTVPNLGPASSNSDECYTNPSFSIGSSIYMFSQEIRKTDCTA | |
| | GEILSIQIVLGRIVDKGQQGPQASPLLVWAVPNPKIINSCAVAAGDEM | |
| | GWVLCSVTLTAASGEPIPHMFDGFWLYKLEPDTEVVSYRITGYAYLL | |
| | DKQYDSVFIGKGGGIQKGNDLYFQMYGLSRNRQSFKALCEHGSCLGT | |
| | GGGGYQVLCDRAVMSFGSEESLITNAYLKVNDLASGKPVIIGQTFPPS | |
| | DSYKGSNGRMYTIGDKYGLYLAPSSWNRYLRFGITPDISVRSTTWLKS | |
| | QDPIMKILSTCTNTDRDMCPEICNTRGYQDIFPLSEDSEYYTYIGITPNN | |
| | GGTKNFVAVRDSDGHIASIDILQNYYSITSATISCFMYKDEIWCIAITEG | |
| | KKQKDNPQRIYAHSYKIRQMCYNMKSATVTVGNAKNITIRRY | |
| 30 | MKVR FENTTSDKGK IPSKVIKSYY GTMDIKKINE GLLDSKILSA | NiVG protein |
| | FNTVIALLGS IVIIVMNIMIIQNYTRSTDN QAVIKDALQG | attachment |
| | IQQQIKGLAD KIGTEIGPKV SLIDTSSTIT IPANIGLLGS | glycoprotein |
| | KISQSTASIN | Truncated A5 |
| | ENVNEKCKFT LPPLKIHECN ISCPNPLPFR EYRPQTEGVS | |
| | NLVGLPNNIC LQKTSNQILK PKLISYTLPV VGQSGTCITD | |
| | PLLAMDEGYF AYSHLERIGS CSRGVSKQRIIGVGEVLDRG | |
| | DEVPSLFMTN VWTPPNPNTV YHCSAVYNNE FYYVLCAVST | |
| | VGDPILNSTY WSGSLMMTRL AVKPKSNGGG YNQHQLALRS | |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QCT | |
| 31 | MSKVIKSYY GTMDIKKINE GLLDSKILSA FNTVIALLGS | NiVG protein |
| | IVIIVMNIMI | attachment |
| | IQNYTRSTDN QAVIKDALQG IQQQIKGLAD KIGTEIGPKV | glycoprotein |
| | SLIDTSSTIT IPANIGLLGS KISQSTASIN ENVNEKCKFT | Truncated A20 |
| | LPPLKIHECN | |
| | ISCPNPLPFR EYRPQTEGVS NLVGLPNNIC LQKTSNQILK | |
| | PKLISYTLPV VGQSGTCITD PLLAMDEGYF AYSHLERIGS | |
| | CSRGVSKQRIIGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE QCT | |
| 32 | MSYY GTMDIKKINE GLLDSKILSA FNTVIALLGS IVIIVMNIMI | NiVG protein |
| | IQNYTRSTDN QAVIKDALQG IQQQIKGLAD KIGTEIGPKV | attachment |
| | SLIDTSSTIT IPANIGLLGS KISQSTASIN ENVNEKCKFT | glycoprotein |
| | LPPLKIHECN | Truncated A25 |
| | ISCPNPLPFR EYRPQTEGVS NLVGLPNNIC LQKTSNQILK | |
| | PKLISYTLPV VGQSGTCITD PLLAMDEGYF AYSHLERIGS | |
| | CSRGVSKQRI IGVGEVLDRG DEVPSLFMTN VWTPPNPNTV | |
| | YHCSAVYNNE FYYVLCAVST VGDPILNSTY WSGSLMMTRL | |
| | AVKPKSNGGG YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV LTVNPLVVNW | |
| | RNNTVISRPG QSQCPRFNTC PEICWEGVYN DAFLIDRINW | |
| | ISAGVFLDSN QTAENPVFTV FKDNEILYRA QLASEDTNAQ | |
| | KTITNCFLLK NKIWCISLVE IYDTGDNVIR PKLFAVKIPE | |
| | QCT | |
| 33 | ILHY EKLSKIGLVK GVTRKYKIKS NPLTKDIVIK MIPNVSNMSQ | Nipah virus |
| | CTGSVMENYK TRLNGILTPI KGALEIYKNQ THDLVGDVRL | NiV-F FO |
| | AGVIMAGVAI GIATAAQITA GVALYEAMKN ADNINKLKSS | T234 |
| | IESTNEAVVK LQETAEKTVY VLTALQDYIN TNLVPTIDKI | truncation (aa |
| | SCKQTELSLD LALSKYLSDL LFVFGPNLQD PVSNSMTIQA | 525-544) AND |
| | ISQAFGGNYE TLLRTLGYAT EDFDDLLESD SITGQIIYVD | mutation on N- |
| | LSSYYIIVRV YFPILTEIQQ AYIQELLPVS FNNDNSEWIS | linked |
| | IVPNFILVRN | glycosylation |
| | TLISNIEIGF CLITKRSVIC NQDYATPMTN NMRECLTGST | site |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK | |
| | KRNTGT | |
| 34 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion |
| | TRLNGILTPI KGALEIYKNN THDLVGDVRL AGVIMAGVAI | glycoprotein |
| | GIATAAQITA GVALYEAMKN ADNINKLKSS IESTNEAWK | (FcDelta22) at |
| | LQETAEKTVY VLTALQDYIN TNLVPTIDKI SCKQTELSLD | cytoplasmic |
| | LALSKYLSDL LFVFGPNLQD PVSNSMTIQA ISQAFGGNYE | tail |
| | TLLRTLGYAT EDFDDLLESD SITGQIIYVD LSSYYIIVRV | (with signal |
| | YFPILTEIQQ AYIQELLPVS FNNDNSEWIS IVPNFILVRN | sequence) |
| | TLISNIEIGF | |
| | CLITKRSVIC NQDYATPMTN NMRECLTGST EKCPRELVVS | |
| | SHVPRFALSN GVLFANCISV TCQCQTTGRA ISQSGEQTLL | |
| | MIDNTTCPTA VLGNVIISLG KYLGSVNYNS EGIAIGPPVF | |
| | TDKVDISSQI SSMNQSLQQS KDYIKEAQRL LDTVNPSLIS | |
| | MLSMIILYVL SIASLCIGLI TFISFIIVEK KRNT | |
| 35 | MKKINEGLLDSKILSA FNTVIALLGS IVIIVMNIMI | NiVG protein |
| | IQNYTRSTDN | |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR | |
| | EYRPQTEGVS NLVGLPNNIC LQKTSNQILK PKLISYTLPV | Truncated and |
| | VGQSGTCITD PLLAMDEGYF AYSHLERIGS CSRGVSKQRI | mutated |
| | IGVGEVLDRG DEVPSLFMTN VWTPPNPNTV YHCSAVYNNE | (E501 A, |
| | FYYVLCAVST VGDPILNSTY WSGSLMMTRL AVKPKSNGGG | W504A, |
| | YNQHQLALRS IEKGRYDKVM PYGPSGIKQG DTLYFPAVGF | Q530A, |
| | LVRTEFKYND SNCPITKCQY SKPENCRLSM GIRPNSHYIL | E533A) NiV G |
| | RSGLLKYNLS DGENPKVVFI EISDQRLSIG SPSKIYDSLG | protein (Gc A |
| | QPVFYQASFS WDTMIKFGDV LTVNPLVVNW RNNTVISRPG | 34) |
| | QSQCPRFNTC PAICAEGVYN DAFLIDRINW ISAGVFLDSN | |
| | ATAANPVFTV FKDNEILYRA QLASEDTNAQ KTITNCFLLK | |
| | NKIWCISLVE IYDTGDNVIR PKLFAVKIPE QCT | |
| 36 | KKINEGLLDSKILSA FNTVIALLGS IVIIVMNIMI IQNYTRSTDN | NiVG protein |
| | QAVIKDALQG IQQQIKGLAD KIGTEIGPKV SLIDTSSTIT | attachment |
| | IPANIGLLGS KISQSTASIN ENVNEKCKFT LPPLKIHECN | glycoprotein |
| | ISCPNPLPFR | Truncated and |
| | EYRPQTEGVS NLVGLPNNIC LQKTSNQILK PKLISYTLPV | mutated |
| | VGQSGTCITD PLLAMDEGYF AYSHLERIGS CSRGVSKQRI | (E501 A, |
| | IGVGEVLDRG DEVPSLFMTN VWTPPNPNTV YHCSAVYNNE | W504A, |
| | FYYVLCAVST VGDPILNSTY WSGSLMMTRL AVKPKSNGGG | Q530A, |
| | YNQHQLALRS IEKGRYDKVM PYGPSGIKQG DTLYFPAVGF | E533A) NiV G |
| | LVRTEFKYND SNCPITKCQY SKPENCRLSM GIRPNSHYIL | protein (Gc A |
| | RSGLLKYNLS DGENPKVVFI EISDQRLSIG SPSKIYDSLG | 34) Without |
| | QPVFYQASFS WDTMIKFGDV LTVNPLVVNW RNNTVISRPG | N-terminal |
| | QSQCPRFNTC PAICAEGVYN DAFLIDRINW ISAGVFLDSN | methionine |
| | ATAANPVFTV FKDNEILYRA QLASEDTNAQ KTITNCFLLK | |
| | NKIWCISLVE IYDTGDNVIR PKLFAVKIPE QCT | |
| 37 | MVVILDKRCY CNLLILILMI SECSVGILHY EKLSKIGLVK | Truncated NiV |
| | GVTRKYKIKS NPLTKDIVIK MIPNVSNMSQ CTGSVMENYK | fusion |
| | TRLNGILTPI KGALEIYKNN THDLVGDVRL AGVIMAGVAI | glycoprotein |
| | GIATAAQITA GVALYEAMKN ADNINKLKSS IESTNEAWK | (FcDelta22) at |
| | LQETAEKTVY VLTALQDYIN TNLVPTIDKI SCKQTELSLD | cytoplasmic |
| | LALSKYLSDL LFVFGPNLQD PVSNSMTIQA ISQAFGGNYE | tail |
| | TLLRTLGYAT EDFDDLLESD SITGQIIYVD LSSYYIIVRV | (with signal |
| | YFPILTEIQQ AYIQELLPVS FNNDNSEWIS IVPNFILVRN | sequence) |
| | TLISNIEIGF | |
| | CLITKRSVIC NQDYATPMTN NMRECLTGST EKCPRELVVS | |
| | SHVPRFALSN GVLFANCISV TCQCQTTGRA ISQSGEQTLL | |
| | MIDNTTCPTA VLGNVIISLG KYLGSVNYNS EGIAIGPPVF | |
| | TDKVDISSQI SSMNQSLQQS KDYIKEAQRL LDTVNPSLIS | |
| | MLSMIILYVL SIASLCIGLI TFISFIIVEK KRNT | |
| 38 | ILHY EKLSKIGLVK GVTRKYKIKS NPLTKDIVIK MIPNVSNMSQ | Nipah virus |
| | CTGSVMENYK TRLNGILTPI KGALEIYKNN THDLVGDVRL | NiV-F FO |
| | AGVIMAGVAI GIATAAQITA GVALYEAMKN ADNINKLKSS | T234 |
| | IESTNEAWK LQETAEKTVY VLTALQDYIN TNLVPTIDKI | truncation (aa |
| | SCKQTELSLD LALSKYLSDL LFVFGPNLQD PVSNSMTIQA | 525-544) |
| | ISQAFGGNYE TLLRTLGYAT EDFDDLLESD SITGQIIYVD | |
| | LSSYYIIVRV YFPILTEIQQ AYIQELLPVS FNNDNSEWIS | |
| | IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK | |
| | KRNTGT | |
| 39 | ILHY EKLSKIGLVK GVTRKYKIKS NPLTKDIVIK MIPNVSNMSQ | Truncated |
| | CTGSVMENYK TRLNGILTPI KGALEIYKNN THDLVGDVRL | mature NiV |
| | AGVIMAGVAI GIATAAQITA GVALYEAMKN ADNINKLKSS | fusion |
| | IESTNEAWK LQETAEKTVY VLTALQDYIN TNLVPTIDKI | glycoprotein |
| | SCKQTELSLD LALSKYLSDL LFVFGPNLQD PVSNSMTIQA | (FcDelta22) at |
| | ISQAFGGNYE TLLRTLGYAT EDFDDLLESD SITGQIIYVD | cytoplasmic |
| | LSSYYIIVRV YFPILTEIQQ AYIQELLPVS FNNDNSEWIS | tail |
| | IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK | |
| | KRNT | |
| 40 | FNTVIALLGS IVIIVMNIMIIQNYTRSTDN QAVIKDALQG | NivG protein |
| | IQQQIKGLAD KIGTEIGPKV SLIDTSSTIT IPANIGLLGS | attachment |
| | KISQSTASIN | glycoprotein |
| | ENVNEKCKFT LPPLKIHECN ISCPNPLPFR EYRPQTEGVS | Without |
| | NLVGLPNNIC LQKTSNQILK PKLISYTLPV VGQSGTCITD | cytoplasmic |
| | PLLAMDEGYF AYSHLERIGS CSRGVSKQRIIGVGEVLDRG | tail |
| | DEVPSLFMTN VWTPPNPNTV YHCSAVYNNE FYYVLCAVST | Uniprot |
| | VGDPILNSTY WSGSLMMTRL AVKPKSNGGG YNQHQLALRS | Q9IH62 |
| | IEKGRYDKVM PYGPSGIKQG DTLYFPAVGF LVRTEFKYND | |
| | SNCPITKCQY SKPENCRLSM GIRPNSHYIL RSGLLKYNLS | |
| | DGENPKVVFI EISDQRLSIG SPSKIYDSLG QPVFYQASFS | |
| | WDTMIKFGDV LTVNPLVVNW RNNTVISRPG QSQCPRFNTC | |
| | PEICWEGVYN DAFLIDRINW ISAGVFLDSN QTAENPVFTV | |
| | FKDNEILYRA QLASEDTNAQ KTITNCFLLK NKIWCISLVE | |
| | IYDTGDNVIR PKLFAVKIPE QC | |
| 41 | MMADSKLVSL NNNLSGKIKD QGKVIKNYYG | Hendra virus |
| | TMDIKKINDG LLDSKILGAF NTVIALLGSI | G protein |
| | IIIVMNIMII QNYTRTTDNQ ALIKESLQSV | Uniprot |
| | QQQIKALTDK IGTEIGPKVS LIDTSSTITI | 089343 |
| | PANIGLLGSK ISQSTSSINE NVNDKCKFTL | |
| | PPLKIHECNI SCPNPLPFRE YRPISQGVSD | |
| | LVGLPNQICL QKTTSTILKP RLISYTLPIN | |
| | TREGVCITDP LLAVDNGFFA YSHLEKIGSC | |
| | TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY | |
| | NQKYIAITKV ERGKYDKVMP YGPSGIKQGD | |
| | TLYFPAVGFL PRTEFQYNDS NCPIIHCKYS | |
| | KAENCRLSMG VNSKSHYILR SGLLKYNLSL | |
| | GGDIILQFIE IADNRLTIGS PSKIYNSLGQ | |
| | PVFYQASYSW DTMIKLGDVD TVDPLRVQWR | |
| | NNSVISRPGQ SQCPRFNVCP EVCWEGTYND | |
| | AFLIDRLNWV SAGVYLNSNQ TAENPVFAVF | |
| | KDNEILYQVP LAEDDTNAQK TITDCFLLEN | |
| | VIWCISLVEI YDTGDSVIRP KLFAVKIPAQ | |
| | CSES | |
| 42 | MMADSKLVSL NNNLSGKIKD QGKVIKNYYG | Hendra virus |
| | TMDIKKINDG LLDSKILGAF NTVIALLGSI | G protein |
| | IIIVMNIMII QNYTRTTDNQ ALIKESLQSV | Uniprot |
| | QQQIKALTDK IGTEIGPKVS LIDTSSTITI | 089343 |
| | PANIGLLGSK ISQSTSSINE NVNDKCKFTL | Without N- |
| | PPLKIHECNI SCPNPLPFRE YRPISQGVSD | terminal |
| | LVGLPNQICL QKTTSTILKP RLISYTLPIN | methionine |
| | TREGVCITDP LLAVDNGFFA YSHLEKIGSC | |
| | TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY | |
| | NQKYIAITKV ERGKYDKVMP YGPSGIKQGD | |
| | TLYFPAVGFL PRTEFQYNDS NCPIIHCKYS | |
| | KAENCRLSMG VNSKSHYILR SGLLKYNLSL | |
| | GGDIILQFIE IADNRLTIGS PSKIYNSLGQ | |
| | PVFYQASYSW DTMIKLGDVD TVDPLRVQWR | |
| | NNSVISRPGQ SQCPRFNVCP EVCWEGTYND | |
| | AFLIDRLNWV SAGVYLNSNQ TAENPVFAVF | |
| | KDNEILYQVP LAEDDTNAQK TITDCFLLEN | |
| | VIWCISLVEI YDTGDSVIRP KLFAVKIPAQ | |
| | CSES | |
| 43 | FNTVIALLGSI | Hendra virus |
| | IIIVMNIMII QNYTRTTDNQ ALIKESLQSV | G protein |
| | QQQIKALTDK IGTEIGPKVS LIDTSSTITI | Uniprot |
| | PANIGLLGSK ISQSTSSINE NVNDKCKFTL | 089343 |
| | PPLKIHECNI SCPNPLPFRE YRPISQGVSD | Without |
| | LVGLPNQICL QKTTSTILKP RLISYTLPIN | cytoplasmic |
| | TREGVCITDP LLAVDNGFFA YSHLEKIGSC | tail |
| | TRGIAKQRII GVGEVLDRGD KVPSMFMTNV | |
| | WTPPNPSTIH HCSSTYHEDF YYTLCAVSHV | |
| | GDPILNSTSW TESLSLIRLA VRPKSDSGDY | |
| | NQKYIAITKV ERGKYDKVMP YGPSGIKQGD | |
| | TLYFPAVGFL PRTEFQYNDS NCPIIHCKYS | |
| | KAENCRLSMG VNSKSHYILR SGLLKYNLSL | |
| | GGDIILQFIE IADNRLTIGS PSKIYNSLGQ | |
| | PVFYQASYSW DTMIKLGDVD TVDPLRVQWR | |
| | NNSVISRPGQ SQCPRFNVCP EVCWEGTYND | |
| | AFLIDRLNWV SAGVYLNSNQ TAENPVFAVF | |
| | KDNEILYQVP LAEDDTNAQK TITDCFLLEN | |
| | VIWCISLVEI YDTGDSVIRP KLFAVKIPAQ | |
| | CSES | |
| 44 | FNTVIALLGSI | Hendra virus |
| | IIIVMNIMII QNYTRTTDNQ ALIKESLQSV | G protein |
| | QQQIKALTDK IGTEIGPKVS LIDTSSTITI | Uniprot |
| | PANIGLLGSK ISQSTSSINE NVNDKCKFTL | 089343 |
| | PPLKIHECNI SCPNPLPFRE YRPISQGVSD | |
| | LVGLPNQICL QKTTSTILKP RLISYTLPIN | |
| | TREGVCITDP LLAVDNGFFA YSHLEKIGSC | |
| | TRGIAKQRII GVGEVLDRGD KVPSMFMTNV | Without |
| | WTPPNPSTIH HCSSTYHEDF YYTLCAVSHV | cytoplasmic |
| | GDPILNSTSW TESLSLIRLA VRPKSDSGDY | tail |
| | NQKYIAITKV ERGKYDKVMP YGPSGIKQGD | |
| | TLYFPAVGFL PRTEFQYNDS NCPIIHCKYS | |
| | KAENCRLSMG VNSKSHYILR SGLLKYNLSL | |
| | GGDIILQFIE IADNRLTIGS PSKIYNSLGQ | |
| | PVFYQASYSW DTMIKLGDVD TVDPLRVQWR | |
| | NNSVISRPGQ SQCPRFNVCP EVCWEGTYND | |
| | AFLIDRLNWV SAGVYLNSNQ TAENPVFAVF | |
| | KDNEILYQVP LAEDDTNAQK TITDCFLLEN | |
| | VIWCISLVEI YDTGDSVIRP KLFAVKIPAQ | |
| | CSES | |
| 45 | MGPAENKKVR FENTTSDKGK IPSKVIKSYY GTMDIKKINE | NiVG protein |
| | GLLDSKILSA FNTVIALLGS IVIIVMNIMIIQNYTRSTDN | attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | (602 aa) |
| | ISCPNPLPFR | |
| | EYRPQTEGVS NLVGLPNNIC LQKTSNQILK PKLISYTLPV | |
| | VGQSGTCITD PLLAMDEGYF AYSHLERIGS CSRGVSKQRI | |
| | IGVGEVLDRG DEVPSLFMTN VWTPPNPNTV YHCSAVYNNE | |
| | FYYVLCAVST VGDPILNSTY WSGSLMMTRL AVKPKSNGGG | |
| | YNQHQLALRS IEKGRYDKVM PYGPSGIKQG DTLYFPAVGF | |
| | LVRTEFKYND SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW RNNTVISRPG | |
| | QSQCPRFNTC PEICWEGVYN DAFLIDRINW ISAGVFLDSN | |
| | QTAENPVFTV FKDNEILYRA QLASEDTNAQ KTITNCFLLK | |
| | NKIWCISLVE IYDTGDNVIR PKLFAVKIPE QC | |
| 46 | MATQEVRLKCLLCGIIVLVLSLEGLGILHYEKLSKIGLVKGITRKYKIK | Hendra virus F |
| | SNPLTKDIVIKMIPNVSNVSKCTGTVMENYKSRLTGILSPIKGAIELYN | Protein |
| | NNTHDLVGDVKLAGVVMAGIAIGIATAAQITAGVALYEAMKNADNI | |
| | NKLKSSIESTNEAVVKLQETAEKTVYVLTALQDYINTNLVPTIDQISCK | |
| | QTELALDLALSKYLSDLLFVFGPNLQDPVSNSMTIQAISQAFGGNYET | |
| | LLRTLGYATEDFDDLLESDSIAGQIVYVDLSSYYIIVRVYFPILTEIQQA | |
| | YVQELLPVSFNNDNSEWISIVPNFVLIRNTLISNIEVKYCLITKKSVICN | |
| | QDYATPMTASVRECLTGSTDKCPRELVVSSHVPRFALSGGVLFANCIS | |
| | VTCQCQTTGRAISQSGEQTLLMIDNTTCTTVVLGNIIISLGKYLGSINY | |
| | NSESIAVGPPVYTDKVDISSQISSMNQSLQQSKDYIKEAQKILDTVNPS | |
| | LISMLSMIILYVLSIAALCIGLITFISFVIVEKKRGNYSRLDDRQVRPVSN | |
| | GDLYYIGT | |
| 47 | ILHYEKLSKIGLVKGITRKYKIKSNPLTKDIVIKMIPNVSNVSKCTGTV | Hendra virus F |
| | MENYKSRLTGILSPIKGAIELYNNNTHDLVGDVKLAGVVMAGIAIGIA | Protein, |
| | TAAQITAGVALYEAMKNADNINKLKSSIESTNEAVVKLQETAEKTVY | Without signal |
| | VLTALQDYINTNLVPTIDQISCKQTELALDLALSKYLSDLLFVFGPNLQ | sequence |
| | DPVSNSMTIQAISQAFGGNYETLLRTLGYATEDFDDLLESDSIAGQIVY | |
| | VDLSSYYIIVRVYFPILTEIQQAYVQELLPVSFNNDNSEWISIVPNFVLIR | |
| | NTLISNIEVKYCLITKKSVICNQDYATPMTASVRECLTGSTDKCPRELV | |
| | VSSHVPRFALSGGVLFANCISVTCQCQTTGRAISQSGEQTLLMIDNTTC | |
| | TTVVLGNIIISLGKYLGSINYNSESIAVGPPVYTDKVDISSQISSMNQSL | |
| | QQSKDYIKEAQKILDTVNPSLISMLSMIILYVLSIAALCIGLITFISFVIVE | |
| | KKRGNYSRLDDRQVRPVSNGDLYYIGT | |
| 48 | MVVILDKRCYCNLLILILMISECSVGILHYEKLSKIGLVKGVTRKYKIK | Nipah virus F |
| | SNPLTKDIVIKMIPNVSNMSQCTGSVMENYKTRLNGILTPIKGALEIYK | Protein |
| | NNTHDLVGDVRLAGVIMAGVAIGIATAAQITAGVALYEAMKNADNI | |
| | NKLKSSIESTNEAVVKLQETAEKTVYVLTALQDYINTNLVPTIDKISCK | |
| | QTELSLDLALSKYLSDLLFVFGPNLQDPVSNSMTIQAISQAFGGNYETL | |
| | LRTLGYATEDFDDLLESDSITGQIIYVDLSSYYIIVRVYFPILTEIQQAYI | |
| | QELLPVSFNNDNSEWISIVPNFILVRNTLISNIEIGFCLITKRSVICNQDY | |
| | ATPMTNNMRECLTGSTEKCPRELVVSSHVPRFALSNGVLFANCISVTC | |
| | QCQTTGRAISQSGEQTLLMIDNTTCPTAVLGNVIISLGKYLGSVNYNSE | |
| | GIAIGPPVFTDKVDISSQISSMNQSLQQSKDYIKEAQRLLDTVNPSLISM | |
| | LSMIILYVLSIASLCIGLITFISFIIVEKKRNTYSRLEDRRVRPTSSGDLYY | |
| | IGT | |
| 49 | ILHYEKLSKIGLVKGVTRKYKIKSNPLTKDIVIKMIPNVSNMSQCTGSV | Nipah virus F |
| | MENYKTRLNGILTPIKGALEIYKNNTHDLVGDVRLAGVIMAGVAIGIA | Protein, |
| | TAAQITAGVALYEAMKNADNINKLKSSIESTNEAVVKLQETAEKTVY | without signal |
| | VLTALQDYINTNLVPTIDKISCKQTELSLDLALSKYLSDLLFVFGPNLQ | sequence |
| | DPVSNSMTIQAISQAFGGNYETLLRTLGYATEDFDDLLESDSITGQIIY | |
| | VDLSSYYIIVRVYFPILTEIQQAYIQELLPVSFNNDNSEWISIVPNFILVR | |
| | NTLISNIEIGFCLITKRSVICNQDYATPMTNNMRECLTGSTEKCPRELV | |
| | VSSHVPRFALSNGVLFANCISVTCQCQTTGRAISQSGEQTLLMIDNTTC | |
| | PTAVLGNVIISLGKYLGSVNYNSEGIAIGPPVFTDKVDISSQISSMNQSL | |
| | QQSKDYIKEAQRLLDTVNPSLISMLSMIILYVLSIASLCIGLITFISFIIVE | |
| | KKRNTYSRLEDRRVRPTSSGDLYYIGT | |
| 50 | MSNKRTTVLIIISYTLFYLNNAAIVGFDFDKLNKIGVVQGRVLNYKIKG | Cedar Virus F |
| | DPMTKDLVLKFIPNIVNITECVREPLSRYNETVRRLLLPIHNMLGLYLN | Protein |
| | NTNAKMTGLMIAGVIMGGIAIGIATAAQITAGFALYEAKKNTENIQKL | |
| | TDSIMKTQDSIDKLTDSVGTSILILNKLQTYINNQLVPNLELLSCRQNKI | |
| | EFDLMLTKYLVDLMTVIGPNINNPVNKDMTIQSLSLLFDGNYDIMMS | |
| | ELGYTPQDFLDLIESKSITGQIIYVDMENLYVVIRTYLPTLIEVPDAQIY | |
| | EFNKITMSSNGGEYLSTIPNFILIRGNYMSNIDVATCYMTKASVICNQD | |
| | YSLPMSQNLRSCYQGETEYCPVEAVIASHSPRFALTNGVIFANCINTIC | |
| | RCQDNGKTITQNINQFVSMIDNSTCNDVMVDKFTIKVGKYMGRKDIN | |
| | NINIQIGPQIIIDKVDLSNEINKMNQSLKDSIFYLREAKRILDSVNISLISP | |
| | SVQLFLIIISVLSFIILLIIIVYLYCKSKHSYKYNKFIDDPDYYNDYKRERI | |
| | NGKASKSNNIYYVGD | |
| 51 | TVLIIISYTLFYLNNAAIVGFDFDKLNKIGVVQGRVLNYKIKGDPMTK | Cedar Virus F |
| | DLVLKFIPNIVNITECVREPLSRYNETVRRLLLPIHNMLGLYLNNTNAK | Protein, |
| | MTGLMIAGVIMGGIAIGIATAAQITAGFALYEAKKNTENIQKLTDSIM | without signal |
| | KTQDSIDKLTDSVGTSILILNKLQTYINNQLVPNLELLSCRQNKIEFDL | sequence |
| | MLTKYLVDLMTVIGPNINNPVNKDMTIQSLSLLFDGNYDIMMSELGY | |
| | TPQDFLDLIESKSITGQIIYVDMENLYVVIRTYLPTLIEVPDAQIYEFNKI | |
| | TMSSNGGEYLSTIPNFILIRGNYMSNIDVATCYMTKASVICNQDYSLP | |
| | MSQNLRSCYQGETEYCPVEAVIASHSPRFALTNGVIFANCINTICRCQD | |
| | NGKTITQNINQFVSMIDNSTCNDVMVDKFTIKVGKYMGRKDINNINIQ | |
| | IGPQIIIDKVDLSNEINKMNQSLKDSIFYLREAKRILDSVNISLISPSVQLF | |
| | LIIISVLSFIILLIIIVYLYCKSKHSYKYNKFIDDPDYYNDYKRERINGKA | |
| | SKSNNIYYVGD | |
| 52 | MALNKNMFSSLFLGYLLVYATTVQSSIHYDSLSKVGVIKGLTYNYKIK | Mojiang virus, |
| | GSPSTKLMVVKLIPNIDSVKNCTQKQYDEYKNLVRKALEPVKMAIDT | Tongguan 1 F |
| | MLNNVKSGNNKYRFAGAIMAGVALGVATAATVTAGIALHRSNENAQ | Protein |
| | AIANMKSAIQNTNEAVKQLQLANKQTLAVIDTIRGEINNNIIPVINQLS | |
| | CDTIGLSVGIRLTQYYSEIITAFGPALQNPVNTRITIQAISSVFNGNFDEL | |
| | LKIMGYTSGDLYEILHSELIRGNIIDVDVDAGYIALEIEFPNLTLVPNAV | |
| | VQELMPISYNIDGDEWVTLVPRFVLTRTTLLSNIDTSRCTITDSSVICDN | |
| | DYALPMSHELIGCLQGDTSKCAREKVVSSYVPKFALSDGLVYANCLN | |
| | TICRCMDTDTPISQSLGATVSLLDNKRCSVYQVGDVLISVGSYLGDGE | |
| | YNADNVELGPPIVIDKIDIGNQLAGINQTLQEAEDYIEKSEEFLKGVNP | |
| | SIITLGSMVVLYIFMILIAIVSVIALVLSIKLTVKGNVVRQQFTYTQHVP | |
| | SMENINYVSH | |
| 53 | IHYDSLSKVGVIKGLTYNYKIKGSPSTKLMVVKLIPNIDSVKNCTQKQ | Mojiang virus, |
| | YDEYKNLVRKALEPVKMAIDTMLNNVKSGNNKYRFAGAIMAGVAL | Tongguan 1 F |
| | GVATAATVTAGIALHRSNENAQAIANMKSAIQNTNEAVKQLQLANK | Protein, |
| | QTLAVIDTIRGEINNNIIPVINQLSCDTIGLSVGIRLTQYYSEIITAFGPAL | without signal |
| | QNPVNTRITIQAISSVFNGNFDELLKIMGYTSGDLYEILHSELIRGNIIDV | sequence |
| | DVDAGYIALEIEFPNLTLVPNAVVQELMPISYNIDGDEWVTLVPRFVL | |
| | TRTTLLSNIDTSRCTITDSSVICDNDYALPMSHELIGCLQGDTSKCARE | |
| | KVVSSYVPKFALSDGLVYANCLNTICRCMDTDTPISQSLGATVSLLDN | |
| | KRCSVYQVGDVLISVGSYLGDGEYNADNVELGPPIVIDKIDIGNQLAG | |
| | INQTLQEAEDYIEKSEEFLKGVNPSIITLGSMVVLYIFMILIAIVSVIALV | |
| | LSIKLTVKGNVVRQQFTYTQHVPSMENINYVSH | |
| 54 | MKKKTDNPTISKRGHNHSRGIKSRALLRETDNYSNGLIVENLVRNCH | Bat |
| | HPSKNNLNYTKTQKRDSTIPYRVEERKGHYPKIKHLIDKSYKHIKRGK | Paramyxovirus |
| | RRNGHNGNIITIILLLILILKTQMSEGAIHYETLSKIGLIKGITREYKVKG | F Protein |
| | TPSSKDIVIKLIPNVTGLNKCTNISMENYKEQLDKILIPINNIIELYANST | |
| | KSAPGNARFAGVIIAGVALGVAAAAQITAGIALHEARQNAERINLLKD | |
| | SISATNNAVAELQEATGGIVNVITGMQDYINTNLVPQIDKLQCSQIKTA | |
| | LDISLSQYYSEILTVFGPNLQNPVTTSMSIQAISQSFGGNIDLLLNLLGY | |
| | TANDLLDLLESKSITGQITYINLEHYFMVIRVYYPIMTTISNAYVQELIK | |
| | ISFNVDGSEWVSLVPSYILIRNSYLSNIDISECLITKNSVICRHDFAMPM | |
| | SYTLKECLTGDTEKCPREAVVTSYVPRFAISGGVIYANCLSTTCQCYQ | |
| | TGKVIAQDGSQTLMMIDNQTCSIVRIEEILISTGKYLGSQEYNTMHVSV | |
| | GNPVFTDKLDITSQISNINQSIEQSKFYLDKSKAILDKINLNLIGSVP | |
| | ISILFIIAILSLILSIITFVIVMIIVRRYNKYTPLINSDPSSRRSTIQD | |
| | VYIIPNPGEHSIRSAARSIDRDRD | |
| 55 | SRALLRETDNYSNGLIVENLVRNCHHPSKNNLNYTKTQKRDSTIPYRV | Bat |
| | EERKGHYPKIKHLIDKSYKHIKRGKRRNGHNGNIITIILLLILILKTQMS | Paramyxovirus |
| | EGAIHYETLSKIGLIKGITREYKVKGTPSSKDIVIKLIPNVTGLNKCTNIS | F Protein, |
| | MENYKEQLDKILIPINNIIELYANSTKSAPGNARFAGVIIAGVALGVAA | without signal |
| | AAQITAGIALHEARQNAERINLLKDSISATNNAVAELQEATGGIVNVIT | sequence |
| | GMQDYINTNLVPQIDKLQCSQIKTALDISLSQYYSEILTVFGPNLQNPV | |
| | TTSMSIQAISQSFGGNIDLLLNLLGYTANDLLDLLESKSITGQITYINLE | |
| | HYFMVIRVYYPIMTTISNAYVQELIKISFNVDGSEWVSLVPSYILIRNSY | |
| | LSNIDISECLITKNSVICRHDFAMPMSYTLKECLTGDTEKCPREAVVTS | |
| | YVPRFAISGGVIYANCLSTTCQCYQTGKVIAQDGSQTLMMIDNQTCSI | |
| | VRIEEILISTGKYLGSQEYNTMHVSVGNPVFTDKLDITSQISNINQSIEQ | |
| | SKFYLDKSKAILDKINLNLIGSVPISILFIIAILSLILSIITFVIVMIIV | |
| | RRYNKYTPLINSDPSSRRSTIQDVYIIPNPGEHSIRSAARSIDRDRD | |
| 56 | MVVILDKRCY CNLLILILMI SECSVG | signal |
| | | sequence |
| 57 | ILHYEKLSKIGLVKGVTRKYKIKSNPLTKDIVIKMIPNVSNMSQCTGSV | Nipah virus |
| | MENYKTRLNGILTPIKGALEIYKNNTHDLVGDVR | NiV-F F2 (aa |
| | | 27-109) |
| 58 | MVVILDKRCYCNLLILILMISECSVGILHYEKLSKIGLVKGVTRKYKIK | Nipah virus F |
| | SNPLTKDIVIKMIPNVSNMSQCTGSVMENYKTRLNGILTPIKGALEIYK | Protein |
| | NNTHDLVGDVRLAGVIMAGVAIGIATAAQITAGVALYEAMKNADNI | |
| | NKLKSSIESTNEAVVKLQETAEKTVYVLTALQDYINTNLVPTIDKISCK | |
| | QTELSLDLALSKYLSDLLFVFGPNLQDPVSNSMTIQAISQAFGGNYETL | |
| | LRTLGYATEDFDDLLESDSITGQIIYVDLSSYYIIVRVYFPILTEIQQAYI | |
| | QELLPVSFNNDNSEWISIVPNFILVRNTLISNIEIGFCLITKRSVICNQDY | |
| | ATPMTNNMRECLTGSTEKCPRELVVSSHVPRFALSNGVLFANCISVTC | |
| | QCQTTGRAISQSGEQTLLMIDNTTCPTAVLGNVIISLGKYLGSVNYNSE | |
| | GIAIGPPVFTDKVDISSQISSMNQSLQQSKDYIKEAQRLLDTVNPSLISM | |
| | LSMIILYVLSIASLCIGLITFISFIIVEKKRNTYSRLEDRRVRPTSSGDLYY | |
| | IGT | |

1. A non-cell particle comprising CD24 or a biologically active portion thereof on an exposed surface of the particle, wherein the non-cell particle is 1 μm or smaller.

2. The non-cell particle of claim 1, wherein the CD24 or the biologically active portion thereof binds Siglec-10.

3. The non-cell particle of claim 1 or claim 2, wherein the CD24 or biologically active portion thereof is human.

4. The non-cell particle of any of claims 1-3, wherein the CD24 or biologically active portion thereof:
(i) comprises the sequence set forth in SEQ ID NO: 2;
(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds Siglec-10; or
(iii) a binding portion of (i) or (ii) that binds to Siglec-10.

5. The non-cell particle of any of claims 1-4, wherein the CD24 or biologically active portion thereof comprises the sequence set forth in SEQ ID NO:2.

6. The non-cell particle of any of claims 1-5, wherein the CD24 or biologically active portion is displayed on an exposed surface of the particle via a Glycosylphosphatidylinositol (GPI) membrane anchor.

7. The non-cell particle of any of claims 1-6, wherein the CD24 or the biologically active portion thereof is encoded by a nucleic molecule encoding the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds Siglec-10.

8. The non-cell particle of any of claims 1-6, wherein the CD24 or the biologically active portion thereof is encoded by a nucleic molecule encoding the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds Siglec-10.

9. The non-cell particle of any of claims 1-5, wherein the CD24 or biologically active portion is displayed on an exposed surface of the particle via a transmembrane domain.

10. The non-cell particle of any of claims 1-9, wherein the CD24 or biologically active portion is a glycoprotein

11. The non-cell particle of any of claims 1-10, wherein the CD24 or biologically active portion is sialylated.

12. The non-cell particle of any of claims 1-11, wherein the CD24 or biologically active portion comprises an α2-3-linked sialoside and/or an α2-6-linked sialoside.

13. The non-cell particle of any of claims 1-12, wherein the CD24 or biologically active portion has a molecular weight of between at or about 35 kDa and at or about 45 kDa.

14. The non-cell particle of any of claims 1-13, wherein the non-cell particle further comprises a CD47 or a biologically active portion thereof on an exposed surface of the non-cell particle.

15. The non-cell particle of claim 14, wherein the CD47 or biologically active portion binds to SIRPα.

16. The non-cell particle of claim 14 or claim 15, wherein the CD47 or biologically active portion is human.

17. The non-cell particle of any one of claims 14-16, wherein the CD47 or biologically active portion comprises the extracellular domain or CD47 or a binding portion thereof that binds to SIRPα.

18. The non-cell particle of any of claims 14-17, wherein the CD47 or biologically active portion thereof:
(i) comprises the sequence set forth in SEQ ID NO: 7;
(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or
(iii) a binding portion of (i) or (ii) that binds to SIRPα.

19. The non-cell particle of any of claims 14-18, wherein the CD47 or biologically active portion thereof comprises the sequence set forth in SEQ ID NO:7.

20. The non-cell particle of any of claims 14-18, wherein the CD47 or biologically active portion thereof comprises the sequence set forth in SEQ ID NO:11.

21. The non-cell particle of any of claims 14-18 and 20, wherein the CD47 or biologically active portion thereof comprises the sequence set forth in SEQ ID NO:9.

22. The non-cell particle of any of claims 14-18 and 20, wherein the CD47 or biologically active portion thereof comprises the sequence set forth in SEQ ID NO:10.

23. The non-cell particle of any of claims 14-18 and 20, wherein the CD47 or biologically active portion thereof comprises the sequence set forth in SEQ ID NO:12.

24. The non-cell particle of any of claims 14-23, wherein the CD47 or biologically active portion is displayed on an exposed surface of the particle via a transmembrane domain.

25. The non-cell particle of any of claims 14-19 and 24, wherein the CD47 or biologically active portion comprises SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα.

26. The non-cell particle of any of claims 14-19, 24 and 25, wherein the CD47 or the biologically active portion thereof is encoded by a nucleic molecule encoding the sequence set forth in SEQ ID NO:5 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5 that binds to SIRPα.

27. The non-cell particle of any of claims 1-26, wherein the non-cell particle is a synthetic particle, a viral particle or a cell-derived particle.

28. The non-cell particle of any of claims 1-27, further comprising a nucleic acid comprising a payload gene encoding an exogenous agent.

29. The non-cell particle of any of claims 1-28, wherein the non-cell particle is a synthetic particle selected from the group consisting of a liposome, a microparticle, a nanoparticle, a nanogel, a dendrimer and a dendrisome.

30. The non-cell particle of any of claims 1-28, wherein the exposed surface is a lipid bilayer and the non-cell particle further comprises a lumen comprising a cytosol, wherein the lumen is surrounded by the lipid bilayer.

31. The non-cell particle of claim 30, wherein the lumen further comprises a nucleic acid comprising a payload gene encoding an exogenous agent.

32. The non-cell particle of claim 30 or claim 31, wherein the non-cell particle is a fusosome and the lipid bilayer further comprises a fusogen.

33. The non-cell particle of any of claims 1-28 and 30-32, wherein the non-cell particle is derived from a source cell.

34. The non-cell particle of any of claims 1-33, wherein the non-cell particle does not comprise a nucleus.

35. The non-cell particle of any of claims 1-28 and 30-32, wherein the non-cell particle is a virus particle or a virus-like particle (VLP).

36. The non-cell particle of claim 35, wherein the virus particle or virus-like particle is a retroviral particle or retrovirus-like particle.

37. The non-cell particle of claim 36, wherein the retroviral particle or retrovirus-like particle is a lentiviral particle or a lentiviral-like particle.

38. The non-cell particle of any of claims 35-37, comprising a fusogen that is a viral fusogen selected from a Class I viral membrane fusion protein, a Class II viral membrane protein, a Class II viral membrane fusion protein, a viral membrane glycoprotein, or a viral envelope protein.

39. The non-cell particle of claim 38, wherein the fusogen is endogenous to the virus.

40. The non-cell particle of claim 38, wherein the fusogen is a pseudotyped fusogen.

41. The non-cell particle of any of claims 32-40, wherein the fusogen is a re-targeted fusogen that binds to a target cell.

42. The non-cell particle of claim 41, wherein the fusogen comprises a targeting moiety that binds to the target cell.

43. The non-cell particle of any of claims 35-42, wherein the virus or virus-like particle further comprising a lumen comprising a nucleic acid.

44. The non-cell particle of claim 43, wherein the nucleic acid comprises a viral nucleic acid comprising one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

45. The non-cell particle of any of claims 35-44, wherein the non-cell particle is a virus-like particle (e.g. retrovirus-like particle) that is a replication defective.

46. A pseudotyped lentivirus or lentiviral-like particle comprising CD24 or a biologically active portion thereof on an exposed surface of the lentiviral particle.

47. The pseudotyped lentivirus or lentiviral-like particle of claim 46, wherein the particle is pseudotyped with a vesicular stomatitis virus envelope glycoprotein (VSV-G).

48. The pseudotyped lentivirus or lentiviral particle of claim 46, wherein the lentiviral particle is pseudotyped with a protein derived from an envelope glycoprotein of a virus of the Paramyxovirus family.

49. The pseudotyped lentivirus or lentiviral-like particle of claim 46, wherein the particle is pseudotyped with a cell targeting fusion protein comprising a protein derived from a Paramyxoviridae envelope protein G or H or a biologically active portion thereof and at least one cell targeting domain.

50. The pseudotyped lentivirus or lentiviral particle of claim 48 or claim 49, wherein the virus of the Paramyxovirus family is a Henipavirus or is a Morbillivirus.

51. The pseudotyped lentivirus or lentiviral particle of any of claims 48-50, wherein the envelope glycoprotein is an envelope glycoprotein G or H or a biologically active portion thereof.

52. The pseudotyped lentivirus or lentiviral-like particle of any of claims 48-51, wherein the envelope glycoprotein is Nipah virus G (Niv-G) protein or a biologically active portion thereof.

53. The pseudotyped lentivirus or lentiviral-like particle of any of claims 48-51, wherein the envelope glycoprotein is a Hendra virus G protein or a biologically active portion thereof.

54. The pseudotyped lentivirus or lentiviral-like particle of any of claims 48-51, wherein the envelope glycoprotein is a measles virus glycoprotein or a biologically active portion thereof.

55. The pseudotyped lentivirus or lentiviral-like particle of any of claims 48-54, wherein said protein derived from an envelope glycoprotein G or H of a virus of the Paramyxoviridae family is at least partially unable to bind at least one natural receptor of said envelope glycoprotein G or H.

56. The pseudotyped lentivirus or lentiviral-like particle of any of claims 48-55, further comprising an F protein molecule or a biologically active portion thereof from a Paramyxovirus.

57. The pseudotyped lentivirus or lentiviral-like particle of claim 56, wherein the Paramyxovirus is a Henipavirus.

58. The pseudotyped lentivirus or lentiviral-like particle of claim 56 or claim 57, wherein the protein protein molecule or a biologically active portion is a NiV-F protein or a biologically active portion.

59. The pseudotyped lentivirus or lentiviral-like particle of any of claims 46-58, wherein the CD24 or biologically active portion thereof:
(i) comprises the sequence set forth in SEQ ID NO: 2;
(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds to Siglec-10; or
(iii) a binding portion of (i) or (ii) that binds to Siglec-10.

60. The pseudotyped lentivirus or lentiviral-like particle of any of claims 46-59, wherein the CD24 or biologically active portion thereof comprises the sequence set forth in SEQ ID NO:2.

61. The pseudotyped lentivirus or lentiviral-like particle of any of claims 46-60, wherein the CD24 or biologically active portion is displayed on an exposed surface of the particle via a Glycosylphosphatidylinositol (GPI) membrane anchor.

62. The pseudotyped lentivirus or lentiviral-like particle of any of claims 46-61, wherein the CD24 or the biologically active portion thereof is encoded by a nucleic molecule encoding the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds to Siglec-10.

63. The pseudotyped lentivirus or lentiviral-like particle of any of claims 46-62, wherein the CD24 or the biologically active portion thereof is encoded by a nucleic molecule encoding the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds to Siglec-10.

64. The pseudotyped lentivirus or lentiviral-like particle of any of claims 46-60, wherein the CD24 or biologically active portion is displayed on an exposed surface of the particle via a transmembrane domain.

65. The pseudotyped lentivirus or lentiviral-like particle of any of claims 46-64, wherein the CD24 or biologically active portion is a glycoprotein

66. The pseudotyped lentivirus or lentiviral-like particle of any of claims 46-65, wherein the CD24 or biologically active portion is sialylated.

67. The pseudotyped lentivirus or lentiviral-like particle of any of claims 46-66, wherein the CD24 or biologically active portion comprises an α2-3-linked sialoside and/or an α2-6-linked sialoside.

68. The pseudotyped lentivirus or lentiviral-like particle of any of claims 46-67, wherein the CD24 or biologically active portion has a molecular weight of between at or about 35 kDa and at or about 45 kDa.

69. The pseudotyped lentivirus or lentiviral-like particle of any of claims 46-68, wherein the non-cell particle further comprises a CD47 or a biologically active portion thereof on an exposed surface of the non-cell particle.

70. The pseudotyped lentivirus or lentiviral-like particle of claim 69, wherein the CD47 or biologically active portion binds to SIRPα.

71. The pseudotyped lentivirus or lentiviral-like particle of claim 69 or claim 70, wherein the CD47 or biologically active portion is human.

72. The pseudotyped lentivirus or lentiviral-like particle of any one of claims 69-71, wherein the CD47 or biologically active portion comprises the extracellular domain or CD47 or a binding portion thereof that binds to SIRPα.

73. The pseudotyped lentivirus or lentiviral-like particle of any of claims 69-72, wherein the CD47 or biologically active portion thereof:
(i) comprises the sequence set forth in SEQ ID NO: 7;
(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or
(iii) a binding portion of (i) or (ii) that binds to SIRPα.

74. The pseudotyped lentivirus or lentiviral-like particle of any of claims 69-73, wherein the CD47 or biologically active portion thereof comprises the sequence set forth in SEQ ID NO:7.

75. The pseudotyped lentivirus or lentiviral-like particle of any of claims 69-73, wherein the CD47 or biologically active portion thereof comprises the sequence set forth in SEQ ID NO:11.

76. The pseudotyped lentivirus or lentiviral-like particle of any of claims 69-73 and 75, wherein the CD47 or biologically active portion thereof comprises the sequence set forth in SEQ ID NO:9.

77. The pseudotyped lentivirus or lentiviral-like particle of any of claims 69-73 and 75, wherein the CD47 or biologically active portion thereof comprises the sequence set forth in SEQ ID NO:10.

78. The pseudotyped lentivirus or lentiviral-like particle of any of claims 69-73 and 77, wherein the CD47 or biologically active portion thereof comprises the sequence set forth in SEQ ID NO:12.

79. The pseudotyped lentivirus or lentiviral-like particle of any of claims 69-78, wherein the CD47 or biologically active portion is displayed on an exposed surface of the particle via a transmembrane domain.

80. The pseudotyped lentivirus or lentiviral-like particle of any of claims 69-74 and 79, wherein the CD47 or biologically active portion comprises SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα.

81. The pseudotyped lentivirus or lentiviral-like particle of any of claims 69-74, 79 and 80, wherein the CD47 or the biologically active portion thereof is encoded by a nucleic molecule encoding the sequence set forth in SEQ ID NO:5 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:5 that binds to SIRPα.

82. The non-cell particle of any of claims 1-45 or the pseudotyped lentivirus or lentiviral-like particle of any of claims 46-81, further comprising a nucleic acid comprising a payload gene encoding an exogenous agent.

83. The non-cell particle of claim 28, claim 31 or claim 82 or the pseudotyped lentivirus or lentiviral-like particle of claim 82, wherein the exogenous agent encodes a therapeutic agent or a diagnostic agent.

84. The non-cell particle of any of claims 1-45, 82 and 83 or the pseudotyped lentivirus or lentiviral-like particle of any of claims 46-83, wherein phagocytosis of the particle by a phagocytic cells, optionally a macrophage, is reduced compared to a reference particle that is otherwise similar but does not comprise the CD24 or biologically active portion, optionally wherein phagocytosis is reduced by at least 10%, 20%, 30%, 40%, 50%, 60%, 70%, 80%, or 90%.

85. The non-cell particle of any of claims 1-45 and 82-84 or the pseudotyped lentivirus or lentiviral-like particle of any of claims 46-84, wherein the half-life of the particle in vivo is increased compared to a reference particle that is otherwise similar but does not comprise the CD24 or biologically active portion, optionally wherein the half-life is increased by at least 10%, 20%, 30%, 40%, 50%, 60%, 70%, 80%, or 90%.

86. A polynucleotide comprising a first nucleic acid sequence encoding CD24 or a biologically active portion and a second nucleic acid encoding CD47 or a biologically active portion.

87. The polynucleotide of claim 86, wherein the encoded CD24 or the biologically active portion thereof binds Siglec-10.

88. The polynucleotide of claim 86 or claim 87, wherein the encoded CD24 or biologically active portion thereof is human.

89. The polynucleotide of any of claims 84-88, wherein the encoded CD24 or biologically active portion thereof:
(i) comprises the sequence set forth in SEQ ID NO: 2;
(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds to Siglec-10; or
(iii) a binding portion of (i) or (ii) that binds to Siglec-10.

90. The polynucleotide of any of claims 84-89, wherein the encoded CD24 or biologically active portion thereof comprises the sequence set forth in SEQ ID NO:2.

91. The polynucleotide of any of claims 84-90, wherein the encoded CD24 or biologically active portion is displayed on an exposed surface of the particle via a Glycosylphosphatidylinositol (GPI) membrane anchor.

92. The polynucleotide of any of claims 84-91, wherein:
the first nucleic acid encoding CD24 or a biologically active portion encodes the sequence set forth in SEQ ID NO:3 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:3 that binds to Siglec-10; or
the first nucleic acid encoding CD24 or a biologically active portion encodes the sequence set forth in SEQ ID NO:15 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:15 that binds to Siglec-10.

93. The polynucleotide of any of claims 84-92, wherein the first nucleic acid encoding CD24 comprises the sequence set forth in SEQ ID NO:4 or a sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:4;

94. The polynucleotide of any of claims 84-93, wherein the encoded CD47 or biologically active portion binds to SIRPα.

95. The polynucleotide of any of claims 84-94, wherein the encoded CD47 or biologically active portion is human.

96. The polynucleotide of any of claims 84-95, wherein the encoded CD47 or biologically active portion comprises the extracellular domain or CD47 or a binding portion thereof that binds to SIRPα.

97. The polynucleotide of any of claims 84-96, wherein the encoded CD47 or biologically active portion thereof:
(i) comprises the sequence set forth in SEQ ID NO: 7;
(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or
(iii) a binding portion of (i) or (ii) that binds to SIRPα.

98. The polynucleotide of any of claims 84-97, wherein the encoded CD47 or biologically active portion thereof comprises the sequence set forth in SEQ ID NO:7.

99. The polynucleotide of any of claims 84-97, wherein the encoded CD47 or biologically active portion thereof comprises the sequence set forth in SEQ ID NO:11.

100. The polynucleotide of any of claims 84-97 and 99, wherein the encoded CD47 or biologically active portion thereof comprises the sequence set forth in SEQ ID NO:9.

101. The polynucleotide of any of claims 84-97 and 99, wherein the encoded CD47 or biologically active portion thereof comprises the sequence set forth in SEQ ID NO:10.

102. The polynucleotide of any of claims 84-97 and 99, wherein the encoded CD47 or biologically active portion thereof comprises the sequence set forth in SEQ ID NO:12.

103. The polynucleotide of any of claims 84-102, wherein the encoded CD47 or biologically active portion comprises a transmembrane domain.

104. The polynucleotide of any of claims 84-103, wherein:
the second nucleic acid encodes the sequence set forth in SEQ ID NO: 5 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:5 that binds to SIRPα; or
the second nucleic acid encodes the sequence set forth in SEQ ID NO: 8 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identity to SEQ ID NO:8 that binds to SIRPα.

105. The polynucleotide of any of claims 84-104, wherein the second nucleic acid encoding CD47 comprises the sequence set forth in SEQ ID NO:13 or a sequence having at least at or about 90%, at least at or about 91%, at or about 92%, at least at or about 93%, at least at or about 94%, at or about 95%, at least at or about 96%, at least at or about 97%, at least at or about 98%, or at least at or about 99% identity to SEQ ID NO:13.

106. The polynucleotide of any of claims 84-105, further comprising at least one promoter that is operatively linked to control expression of the CD24 or biologically active portion and/or the CD47 or biologically active portion.

107. The polynucleotide of any of claims 84-106, wherein the first and second nucleic acid are operatively linked to the same promoter.

108. The polynucleotide of any of claims 84-106, wherein the first nucleic acid is operatively linked to a first promoter and the second nucleic acid is operatively linked to a second promoter.

109. The polynucleotide of claim 108, wherein the first and second promoter are different.

110. The polynucleotide of any of claims 106-109, wherein the promoter, or each promoter individually, is a heterologous promoter.

111. The polynucleotide of any of claims 106-110, wherein the promoter, or each promoter individually, is an inducible promoter.

112. The polynucleotide of any of claims 84-111, further comprising a nucleic acid sequence encoding a linking peptide between the first and second nucleic acid sequences, wherein the linking peptide separates the translation products of the first and second nucleic acid sequences during or after translation.

113. The polynucleotide of claim 112, wherein the linking peptide comprises an internal ribosome entry site (IRES), a self-cleaving peptide, or a peptide that causes ribosome skipping, optionally a T2A peptide.

114. A vector, comprising the polynucleotide of any of claims 84-112.

115. The vector of claim 114, wherein the vector is a mammalian vector, viral vector or artificial chromosome, optionally wherein the artificial chromosome is a bacterial artificial chromosome (BAC).

116. A method of making a non-cell particle comprising CD24 or a biologically active portion, comprising:
a) providing a cell that comprises a nucleic acid encoding CD24 or a biologically active portion thereof;
b) culturing the cell under conditions that allow for production of a non-cell particle, and
c) separating, enriching, or purifying the particle from the cell, thereby making the fusosome.

117. The method of claim 116, wherein the cell further comprises a nucleic acid encoding CD47 or a biologically active portion thereof or the nucleic acid further encodes CD47 or a biologically active portion thereof.

118. A method of making a non-cell particle comprising CD24 or a biologically active portion, comprising:
a) providing a cell that comprises the polynucleotide of any of claims 84-112 or the vector of claim 114 or claim 115;
b) culturing the cell under conditions that allow for production of a non-cell particle, and
c) separating, enriching, or purifying the non-cell particle from the cell, thereby making the fusosome.

119. The method of any of claims 116-118, wherein the cell is a mammalian cell and the non-cell particle is a vesicle or an exosome, optionally wherein the vesicle is a microvesicle or a nanovesicle.

120. The method of any of claims 116-118, wherein the cell is a producer cell and the non-cell particle is a viral particle or a viral-like particle, optionally a retroviral particle or a retroviral-like particle, optionally a lentiviral particle or lentiviral-like particle.

121. The method of any of claims 116-120, wherein the cell further comprises an exogenous nucleic acid sequence encoding an exogenous agent.

122. The method of any of claims 116-121, wherein the cell further comprises a fusogen.

123. A non-cell particle made by the method of any of claims 116-122.

124. A mammalian cell comprising (i) a viral nucleic acid(s) and (ii) nucleic acid encoding an exogenous CD24 or a biologically active portion thereof, optionally wherein the viral nucleic acid(s) are lentiviral nucleic acids.

125. The mammalian cell of claim 124, wherein the viral nucleic acid(s) lacks one or more genes involved in viral replication.

126. The mammalian cell of claim 124 or claim 125, wherein the viral nucleic acid comprises:
one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3);
a nucleic acid encoding a viral envelope protein; and/or
a nucleic acid encoding a viral packaging protein selected from one or more of Gag, Pol, Rev and Tat.

127. The mammalian cell of any of claims 124-126, wherein the the exogenous CD24 or biologically active portion comprises:
(i) the sequence set forth in SEQ ID NO: 2;
(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:2 that binds Siglec-10; or
(iii) a binding portion of (i) or (ii) that binds to Siglec-10.

128. The mammalian cell of claim 127, wherein the nucleic acid encoding exogenous CD24 further encodes a a Glycosylphosphatidylinositol (GPI) membrane anchor signal sequence or a transmembrane domain.

129. The mammalian cell of any of claims 124-128, wherein the cell further comprises a nucleic acid encoding exogenous CD47 or a biologically active portion.

130. The mammalian cell of claim 129, wherein the exogenous CD47 or biologically active portion comprises:
(i) the sequence set forth in SEQ ID NO: 7;
(ii) an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:7 that binds to SIRPα; or
(iii) a binding portion of (i) or (ii) that binds to SIRPα.

131. The mammalian cell of claim 130, wherein the nucleic acid encoding exogenous CD47 further encodes a Glycosylphosphatidylinositol (GPI) membrane anchor signal sequence or a transmembrane domain.

132. The mammalian cell of any of claims 124-131, wherein the nucleic acid encoding the exogenous CD24 or biologically active portion and the nucleic acid encoding the exogenous CD47 or biologically active portion are encoded by the polynucleotide of any of claims 84-112.

133. A viral vector particle or viral-like particle produced from the mammalian cell of any of claims 124-132.

134. A composition comprising a plurality of non-cell particles of any of claims 1-45.

135. A composition comprising a plurality of pseudotyped lentivirus or lentiviral-like particles of any of claims 46-81.

136. The composition of claim 134 or claim 135 further comprising a pharmaceutically acceptable carrier.

137. The pharmaceutical composition of any of claims 134-136, wherein the plurality of particles comprise an average diameter of less than 1 μm.

138. A method of delivering an exogenous agent to a subject (e.g., a human subject), the method comprising administering to the subject the non-cell particle of any of claims 1-45 or the pseudotyped lentivirus or lentiviral-like particles of any of claims 46-81 or the composition of any of claims 134-137.

139. A method of treating a disease or disorder in a subject (e.g., a human subject), the method comprising administering to the subject a non-cell particle of any of claims 1-45 or the pseudotyped lentivirus or lentiviral-like particles of any of claims 46-81 or the composition of any of claims 134-137.

140. A method of evading phagocytosis of a particle by a phagocytic cell, the method comprising contacting a phagocytic cell with a non-cell particle of any of claims 1-45 or the pseudotyped lentivirus or lentiviral-like particles of any of claims 46-81 or the composition of any of claims 134-137, whereby said particles evades phacocytosis by said phagocytic cell.

141. A method of increasing the life of a particle in vivo in a mammal, method comprising administering the non-cell particle of any of claims 1-45 or the pseudotyped lentivirus or lentiviral-like particles of any of claims 46-81 or the composition of any of claims 134-137 to a mammalian subject wherein said administered particles have a longer half-life in said mammal than an otherwise similar particle that does not have CD24 expressed thereon.

142. The non-cell particle of any of claims 1-27, 29, 30, and 32-45, further comprising an exogenous agent.

143. The non-cell particle of any of claims 28, 31, 82, 83, and 142, wherein the exogenous agent comprises a protein.

144. The non-cell particle of any of claims 28, 31, 82, 83, 142, and 143, wherein the exogenous agent comprises a membrane protein.

145. The non-cell particle of any of claims 28, 31, 82, 83, and 142-144, wherein the exogenous agent comprises a chimeric antigen receptor (CAR), a T cell receptor, an integrin, an ion channel, a pore forming protein, a Toll-Like Receptor, an interleukin receptor, a cell adhesion protein, or a transport protein.

146. The non-cell particle of any of claims 28, 31, 82, 83, and 142-145, wherein the exogenous agent comprises a CAR comprising an antigen binding domain, a transmembrane domain, and one or more signaling domains.

147. The non-cell particle of claim 146, wherein the antigen binding domain binds to a surface antigen characteristic of a cell type or a disorder.

148. The non-cell particle of claim 146 or 147, wherein the antigen binding domain binds to a surface antigen characteristic of a neoplastic cell, a T cell, an autoimmune or inflammatory disorder, a senescent cell, or an infectious disease.

149. A method of delivering an exogenous agent to a subject (e.g., a human subject), the method comprising administering to a subject the non-cell particle of any of claims 28, 31, 82, 83, and 142-148, wherein the payload gene encoding the exogenous agent or the exogenous agent is delivered to a target cell.

150. The method of claim 149, wherein the exogenous agent is a CAR.

151. The method of claim 149 or claim 150, wherein the target cell is a T cell.

152. The method of any of claims 149-151, wherein the target cell is any of a CD4+ T cell, a CD8+T cell, an alpha beta T cell, a gamma delta T cell, a naive T cell, an effector T cell, a cytotoxic T cell (e.g., a CD8+ cytotoxic T cell), a regulatory T cell (e.g., a thymus-derived regulatory T cell, a peripherally derived regulatory T cell, a CD4+Foxp3+ regulatory T cell, or a CD4+FoxP3− type 1 regulatory T (Tr1) cell), a helper T cell (e.g., a CD4+ helper T cell, a Th1 cell, a Th2 cell, a Th3 cell, a Th9 cell, a Th17 cell, a Th22 cell, or a T follicular helper (Tfh) cell), a memory T cell (e.g., a stem cell memory T cell, a central memory T cell, or an effector memory T cell), a NKT cell, and a Mucosal associated invariant T (MAIT) cell.
